(19) United States
(12) Patent Application Publication
(10) Pub. No.: US 2022/0380773 A1
(43) Pub. Date: Dec. 1, 2022

(54) COMPOUNDS AND METHODS FOR REDUCING APP EXPRESSION

(71) Applicant: Ionis Pharmaceuticals, Inc., Carlsbad, CA (US)

(72) Inventors: Susan M. Freier, San Diego, CA (US); Huynh-Hoa Bui, San Diego, CA (US); Holly Kordasiewicz, San Diego, CA (US); Eric E. Swayze, Encinitas, CA (US)

(73) Assignee: Ionis Pharmaceuticals, Inc., Carlsbad, CA (US)

(21) Appl. No.: 17/424,672

(22) PCT Filed: Jan. 29, 2020
(86) PCT No.: PCT/US2020/015701

§ 371 Date: Jul. 21, 2021

Related U.S. Application Data
(60) Provisional application No. 62/798,353, filed on Jan. 29, 2019.
(60) Provisional application No. 62/841,169, filed on Apr. 30, 2019.
(60) Provisional application No. 62/915,764, filed on Oct. 16, 2019.

(51) Int. Cl. C12N 15/113

(52) U.S. Cl. CPC C12N 15/1138; C12N 2310/315; C12N 2310/3341; C12N 2310/341; C12N 2310/321; C12N 2310/14

(57) ABSTRACT

Provided are compounds, methods, and pharmaceutical compositions for reducing the amount or activity of APP RNA in a cell or animal, and in certain instances reducing the amount of APP protein in a cell or animal Such compounds, methods, and pharmaceutical compositions are useful to ameliorate at least one symptom or hallmark of a neurodegenerative disease. Such symptoms and hallmarks include cognitive impairment, including a decline in memory and language skills, behavioral and psychological symptoms such as apathy and lack of motivation, gait disturbances and seizures, progressive dementia, and abnormal amyloid deposits.

### SEQUENCE LISTING

The present application is being filed along with a Sequence Listing in electronic format. The Sequence Listing is provided as a file entitled BIOL0351WOSEQ_ST25.txt, created on Jan. 22, 2020, which is 580 KB in size. The information in the electronic format of the sequence listing is incorporated herein by reference in its entirety.

### FIELD

Provided are compounds, methods, and pharmaceutical compositions for reducing the amount or activity of APP RNA in a cell or animal, and in certain instances reducing the amount of APP protein in a cell or animal. Certain such compounds, methods, and pharmaceutical compositions are useful to ameliorate at least one symptom or hallmark of a neurodegenerative disease. Such symptoms and hallmarks include cognitive impairment, including a decline in memory and language skills, behavioral and psychological symptoms such as apathy and lack of motivation, gait disturbances and seizures, progressive dementia, and abnormal amyloid deposits. Such neurodegenerative diseases include Alzheimer's Disease, Alzheimer's Disease in Down Syndrome patients, and Cerebral Amyloid Angiopathy.

### BACKGROUND

Alzheimer's Disease (AD) is the most common cause of age-associated dementia, affecting an estimated 5.7 million Americans a year (Alzheimer's Association. 2018 Alzheimer's Disease Facts and Figures. Alzheimer's Dement. 2018; 14(3):367-429). AD is characterized by the accumulation of β-amyloid plaques in the brain prior to the onset of overt clinical symptoms. Such overt clinical symptoms include cognitive impairment, including a decline in memory and language skills, behavioral and psychological symptoms such as apathy and lack of motivation, gait disturbances and seizures, and progressive dementia.

Patients with Down Syndrome (DS) can experience early-onset Alzheimer's disease (AD in DS), with amyloid plaque formation observed by age 40 in most DS patients, and Alzheimer's dementia observed by age 50 in more than 50% of Down syndrome patients.

Cerebral Amyloid Angiopathy (CAA) is a related disease that is characterized by the deposition of β-amyloid in blood vessels of the CNS. CAA is often observed in AD patients upon autopsy, but is also associated with aging in the absence of clinical signs of AD.

AD, AD in DS, and CAA are all characterized by the abnormal accumulation of β-amyloid plaques. β-amyloid (Aβ) is derived from amyloid precursor protein (APP) upon processing of APP by α-, β-, and γ-secretases. In addition to the 42-amino acid fragment Aβ, a variety of other fragments of APP are also formed, several of which are proposed to contribute to the onset of dementia in AD (reviewed in Nhan, et al., “The multifaceted nature of amyloid precursor protein and its proteolytic fragments: friends and foes”, Acta Neuropath., 2015, 129(1):1-19). The increased incidence of AD in DS patients is thought to be directly related to the increased copy number of the APP gene, which resides on chromosome 21.

Certain RNAi compounds have been described. RNAi compounds interact with the RNA silencing complex (RISC), ultimately resulting in cleavage of the target nucleic acid. See, e.g., Sharp et al., 2001, Genes Dev. 15: 485; Bernstein, et al., 2001, Nature, 409: 363; Nykanen, et al., 2001, Cell, 107: 309; Elbashir, et al., 2001, Genes Dev. 15: 188; Lima et al., (2012) Cell 150: 883-894.

Currently there is a lack of acceptable options for treating neurodegenerative diseases such as AD, AD in DS, and CAA. It is therefore an object herein to provide compounds, methods, and pharmaceutical compositions for the treatment of such diseases.

### SUMMARY OF THE INVENTION

Provided herein are compounds, methods and pharmaceutical compositions for reducing the amount or activity of APP RNA, and in certain embodiments reducing the amount of APP protein in a cell or animal. In certain embodiments, the animal has a neurodegenerative disease. In certain embodiments, the animal has Alzheimer's Disease (AD). In certain embodiments, the animal has Alzheimer's Disease in conjunction with Down Syndrome (AD in DS). In certain embodiments, the animal has Cerebral Amyloid Angiopathy (CAA). In certain embodiments, compounds useful for reducing expression of APP RNA are oligomeric compounds. In certain embodiments, compounds useful for reducing expression of APP RNA are modified oligonucleotides.

Also provided are methods useful for ameliorating at least one symptom or hallmark of a neurodegenerative disease. In certain embodiments, the neurodegenerative disease is Alzheimer's Disease. In certain embodiments, the neurodegenerative disease is Alzheimer's Disease in Down Syndrome patients. In certain embodiments, the neurodegenerative disease is Cerebral Amyloid Angiopathy (CAA). In certain embodiments, the symptom or hallmark includes cognitive impairment, including a decline in memory and language skills, behavioral and psychological symptoms such as apathy and lack of motivation, gait disturbances and seizures, progressive dementia, or abnormal amyloid deposits.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D configuration as found in naturally occurring deoxyribonucleic acids (DNA). In certain embodiments, a 2′-deoxynucleoside or a nucleoside comprising an unmodified 2′-deoxyribosyl sugar moiety may comprise a modified nucleobase or may comprise an RNA nucleobase (uracil).

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “3′ target site” refers to the 3′-most nucleotide of a target nucleic acid which is complementary to an antisense oligonucleotide, when the antisense oligonucleotide is hybridized to the target nucleic acid.

As used herein, “5′ target site” refers to the 5′-most nucleotide of a target nucleic acid which is complementary to an antisense oligonucleotide, when the antisense oligonucleotide is hybridized to the target nucleic acid.

As used herein, “5-methyl cytosine” means a cytosine modified with a methyl group attached to the 5 position. A 5-methyl cytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “administration” or “administering” means providing a pharmaceutical agent or composition to an animal.

As used herein, “animal” means a human or non-human animal.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “antisense oligonucleotide” means an oligonucleotide, including the oligonucleotide portion of an oligomeric compound that is complementary to a target nucleic acid and is capable of achieving at least one antisense activity. Antisense oligonucleotides include but are not limited to antisense RNAi oligonucleotides and antisense RNase H oligonucleotides.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is cognitive impairment, including a decline in memory and language skills, behavioral and psychological symptoms such as apathy and lack of motivation, gait disturbances and seizures, progressive dementia, or abnormal amyloid deposits.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “blunt” or “blunt ended” in reference to a duplex formed by two oligonucleotides mean that there are no terminal unpaired nucleotides (i.e. no overhanging nucleotides). One or both ends of a double-stranded RNAi compound can be blunt.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Certain modified nucleobases that pair with natural nucleobases or with other modified nucleobases are known in the art. For example, inosine can pair with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt modified sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides.

As used herein, “double-stranded” means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “duplex” or “duplex region” means the structure formed by two oligonucleotides or portions thereof that are hybridized to one another.

As used herein, “gapmer” means a modified oligonucleotide comprising an internal region having a plurality of nucleosides that support RNase H cleavage positioned between external regions having one or more nucleosides, wherein at least one of the nucleosides comprising the internal region is chemically distinct from at least one nucleoside of each of the external regions. Specifically, the nucleosides that define the boundaries of the internal region and each external region must be chemically distinct. The internal region may be referred to as the “gap” and the external regions may be referred to as the “wings.” Unless otherwise indicated, “gapmer” refers to a sugar motif. In certain embodiments, the sugar moiety of each nucleoside of the gap is a 2′-β-D-deoxyribosyl sugar moiety. In certain embodiments, the gap comprises one 2′-substituted nucleoside at position 1, 2, 3, 4, or 5 of the gap, and the remainder of the nucleosides of the gap are 2′-β-D-deoxynucleosides. Unless otherwise indicated, a gapmer may comprise one or more modified internucleoside linkages and/or modified nucleobases and such modifications do not necessarily follow the gapmer pattern of the sugar modifications. As used herein, the term “mixed gapmer” indicates a gapmer having a gap comprising 2′-β-D-deoxynucleosides and wings comprising modified nucleosides comprising at least two different sugar modifications.

As used herein, “hotspot region” is a range of nucleobases on a target nucleic acid that is amenable to oligomeric compound-mediated reduction of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

“Lipid nanoparticle” or “LNP” is a vesicle comprising a lipid layer encapsulating a pharmaceutically active molecule, such as a nucleic acid molecule, e.g., an RNAi or a plasmid from which an RNAi is transcribed. LNPs are described in, for example, U.S. Pat. Nos. 6,858,225, 6,815,432, 8,158,601, and 8,058,069, the entire contents of which are hereby incorporated herein by reference.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned.

As used herein, “MOE” means O-methoxyethyl. “2′-MOE” or “2′-MOE modified sugar” means a 2′-OCH₂CH₂OCH₃ group in place of the 2′—OH group of a ribosyl sugar moiety. As used herein, “2′-MOE nucleoside” means a nucleoside comprising a 2′-MOE sugar moiety.

As used herein, “motif” means the pattern of unmodified and/or modified sugar moieties, nucleobases, and/or internucleoside linkages, in an oligonucleotide.

As used herein, “neurodegenerative disease” means a condition marked by progressive loss of function or structure, including loss of neuronal function and death of neurons. In certain embodiments, the neurodegenerative disease is Alzheimer's Disease. In certain embodiments, the neurodegenerative disease is Alzheimer's Disease in Down Syndrome patients. In certain embodiments, the neurodegenerative disease is Cerebral Amyloid Angiopathy.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methyl cytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “nucleoside overhang” refers to unpaired nucleotides at either or both ends of a duplex formed by hybridization of an antisense RNAi oligonucleotide and a sense RNAi oligonucleotide.

As used herein, “modified nucleoside” means a nucleoside comprising a modified nucleobase and/or a modified sugar moiety.

As used herein, “linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a polymer of linked nucleosides connected via internucleoside linkages, wherein each nucleoside and internucleoside linkage may be modified or unmodified. Unless otherwise indicated, oligonucleotides consist of 8-50 linked nucleosides. An oligonucleotide may be paired with a second oligonucleotide that is complementary to the oligonucleotide or it may be unpaired. A “single-stranded oligonucleotide” is an unpaired oligonucleotide. A “double-stranded oligonucleotide” is an oligonucleotide that is paired with a second oligonucleotide. An “oligonucleotide duplex” means a duplex formed by two paired oligonucleotides having complementary nucleobase sequences. Each oligo of an oligonucleotide duplex is a “duplexed oligonucleotide” or a “double-stranded oligonucleotide”.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. Thus, each nucleoside of an unmodified oligonucleotide is a DNA or RNA nucleoside and each internucleoside linkage is a phosphodiester linkage.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein “prodrug” means a therapeutic agent in a first form outside the body that is converted to a second form within an animal or cells thereof. Typically, conversion of a prodrug within the animal is facilitated by the action of an enzymes (e.g., endogenous or viral enzyme) or chemicals present in cells or tissues and/or by physiologic conditions. In certain embodiments, the first form of the prodrug is less active than the second form.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. RNAi compounds may comprise conjugate groups and/or terminal groups. In certain embodiments, an RNAi compound modulates the amount, activity, and/or splicing of a target nucleic acid. The term RNAi compound excludes antisense compounds that act through RNase H.

As used herein, “RNAi oligonucleotide” means an antisense RNAi oligonucleotide or a sense RNAi oligonucleotide.

As used herein, “antisense RNAi oligonucleotide” means an oligonucleotide comprising a region that is complementary to a target sequence, and which includes at least one chemical modification suitable for RNAi.

As used herein, “sense RNAi oligonucleotide” means an oligonucleotide comprising a region that is complementary to a region of an antisense RNAi oligonucleotide, and which is capable of forming a duplex with such antisense RNAi oligonucleotide. A duplex formed by an antisense RNAi oligonucleotide and a sense RNAi oligonucleotide is referred to as a double-stranded RNAi compound (dsRNAi) or a short interfering RNA (siRNA).

As used herein, “RNase H compound” means an antisense compound that acts, at least in part, through RNase H to modulate a target nucleic acid and/or protein encoded by a target nucleic acid. In certain embodiments, RNase H compounds are single-stranded. In certain embodiments, RNase H compounds are double-stranded. RNase H compounds may comprise conjugate groups and/or terminal groups. In certain embodiments, an RNase H compound modulates the amount or activity of a target nucleic acid. The term RNase H compound excludes antisense compounds that act principally through RISC/Ago2.

As used herein, “antisense RNase H oligonucleotide” means an oligonucleotide comprising a region that is complementary to a target sequence, and which includes at least one chemical modification suitable for RNase H-mediated nucleic acid reduction.

As used herein, “self-complementary” in reference to an oligonucleotide means an oligonucleotide that at least partially hybridizes to itself.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “stabilized phosphate group” means a 5′-phosphate analog that is metabolically more stable than a 5′-phosphate as naturally occurs on DNA or RNA.

As used herein, “standard cell assay” means the assay described in Examples 1 or 5 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an antisense compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent or composition that provides a therapeutic benefit to an animal. For example, a therapeutically effective amount improves a symptom of a disease.

### CERTAIN EMBODIMENTS

The present disclosure provides the following non-limiting numbered embodiments:
Embodiment 1. An oligomeric compound comprising a modified oligonucleotide consisting of 12 to 30 linked nucleosides wherein the nucleobase sequence of the modified oligonucleotide is at least 80% complementary to an equal length portion of a APP RNA, and wherein the modified oligonucleotide comprises at least one modification selected from a modified sugar, a sugar surrogate, and a modified internucleoside linkage.
Embodiment 2. An oligomeric compound comprising a modified oligonucleotide consisting of 12 to 30 linked nucleosides and having a nucleobase sequence comprising at least 12, 13, 14, 15, 16, 17, or 18 nucleobases of any of SEQ ID NOS: 12-501 Embodiment 3. An oligomeric compound comprising a modified oligonucleotide consisting of 12 to 30 linked nucleosides and having a nucleobase sequence comprising at least 12, 13, 14, 15, 16, 17, 18, 19, or 20 nucleobases of any of SEQ ID NOS: 502-516.
Embodiment 4. An oligomeric compound comprising a modified oligonucleotide consisting of 12 to 30 linked nucleosides and having a nucleobase sequence complementary to at least 8, at least 9, at least 10, at least 11, at least 12, at least 13, at least 14, at least 15, at least 16, at least 17, at least 18, at least 19, or at least 20 contiguous nucleobases of:
   an equal length portion of nucleobases 40-78 of SEQ ID NO: 1;
   an equal length portion of nucleobases 69-146 of SEQ ID NO: 1;
   an equal length portion of nucleobases 83-129 of SEQ ID NO: 1;
   an equal length portion of nucleobases 194-231 of SEQ ID NO: 1;
   an equal length portion of nucleobases 194-238 of SEQ ID NO: 1;
   an equal length portion of nucleobases 236-268 of SEQ ID NO: 1;
   an equal length portion of nucleobases 258-284 of SEQ ID NO: 1;
   an equal length portion of nucleobases 285-311 of SEQ ID NO: 1;
   an equal length portion of nucleobases 296-321 of SEQ ID NO: 1;
   an equal length portion of nucleobases 307-330 of SEQ ID NO: 1;
   an equal length portion of nucleobases 339-383 of SEQ ID NO: 1;
   an equal length portion of nucleobases 415-439 of SEQ ID NO: 1;
   an equal length portion of nucleobases 415-477 of SEQ ID NO: 1;
   an equal length portion of nucleobases 477-506 of SEQ ID NO: 1;
   an equal length portion of nucleobases 477-523 of SEQ ID NO: 1;
   an equal length portion of nucleobases 477-541 of SEQ ID NO: 1;
   an equal length portion of nucleobases 530-557 of SEQ ID NO: 1;
   an equal length portion of nucleobases 636-661 of SEQ ID NO: 1;
   an equal length portion of nucleobases 652-697 of SEQ ID NO: 1;
   an equal length portion of nucleobases 920-950 of SEQ ID NO: 1;
   an equal length portion of nucleobases 1152-1179 of SEQ ID NO: 1;
   an equal length portion of nucleobases 1227-1265 of SEQ ID NO: 1;
   an equal length portion of nucleobases 1227-1274 of SEQ ID NO: 1;
   an equal length portion of nucleobases 1518-1543 of SEQ ID NO: 1;
   an equal length portion of nucleobases 1531-1593 of SEQ ID NO: 1;
   an equal length portion of nucleobases 1544-1593 of SEQ ID NO: 1;
   an equal length portion of nucleobases 1635-1657 of SEQ ID NO: 1;
   an equal length portion of nucleobases 1778-1800 of SEQ ID NO: 1;
   an equal length portion of nucleobases 1882-1908 of SEQ ID NO: 1; or
   an equal length portion of nucleobases 2051-2074 of SEQ ID NO: 1.
Embodiment 5. The oligomeric compound of any of embodiments 1-4, wherein the modified oligonucleotide has a nucleobase sequence that is at least 80%, 85%, 90%, 95%, or 100% complementary to any of the nucleobase sequences of SEQ ID NO: 1-7 when measured across the entire nucleobase sequence of the modified oligonucleotide.
Embodiment 6. The oligomeric compound of any of embodiments 1-5, wherein the modified oligonucleotide comprises at least one modified nucleoside.
Embodiment 7. The oligomeric compound of embodiment 6, wherein the modified oligonucleotide comprises at least one modified nucleoside comprising a modified sugar moiety.
Embodiment 8. The oligomeric compound of embodiment 7, wherein the modified oligonucleotide comprises at least one modified nucleoside comprising a bicyclic sugar moiety.
Embodiment 9. The oligomeric compound of embodiment 8, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.
Embodiment 10. The oligomeric compound of any of embodiments 5-9, wherein the modified oligonucleotide comprises at least one modified nucleoside comprising a non-bicyclic modified sugar moiety.
Embodiment 11. The oligomeric compound of embodiment 7, wherein the modified oligonucleotide comprises at least one nucleoside comprising a bicyclic sugar moiety having a 2′-4′ bridge and at least one nucleoside comprising a non-bicyclic modified sugar moiety.
Embodiment 12. The oligomeric compound of embodiment 10 or 11, wherein the non-bicyclic modified sugar moiety is a 2′-MOE modified sugar moiety or a 2′-OMe modified sugar moiety.
Embodiment 13. The oligomeric compound of embodiment 11, wherein the bicyclic modified sugar moiety has a 2′-4′ bridge, wherein the 2′-4′ bridge is selected from —O—CH₂ and —O—CH(CH₃)—.
Embodiment 14. The oligomeric compound of any of embodiments 1-13, wherein the modified oligonucleotide comprises at least one modified nucleoside comprising a sugar surrogate.
Embodiment 15. The oligomeric compound of embodiment 14, wherein the modified oligonucleotide comprises at least one modified nucleoside comprising a sugar surrogate selected from morpholino and PNA.
Embodiment 16. The oligomeric compound of any of embodiments 1-13, wherein the modified oligonucleotide has a sugar motif comprising:
   a 5′-region consisting of 1-5 linked 5′-region nucleosides;
   a central region consisting of 6-10 linked central region nucleosides; and
   a 3′-region consisting of 1-5 linked 3′-region nucleosides; wherein
   each of the 5′-region nucleosides and each of the 3′-region nucleosides comprises a modified sugar moiety and each of the central region nucleosides comprises a 2′-β-D-deoxyribosyl sugar moiety.
Embodiment 17. The oligomeric compound of embodiment 16, wherein the modified oligonucleotide has a sugar motif comprising:
   a 5′-region consisting of 5 linked 5′-region nucleosides;
   a central region consisting of 10 linked central region nucleosides; and
   a 3′-region consisting of 5 linked 3′-region nucleosides; wherein
   each of the 5′-region nucleosides and each of the 3′-region nucleosides comprises either a cEt modified sugar moiety or a 2′-MOE modified sugar moiety, and each of the central region nucleosides comprises a 2′-β-D-deoxyribosyl sugar moiety.
Embodiment 18. The oligomeric compound of any of embodiments 1-17, wherein the modified oligonucleotide comprises at least one modified internucleoside linkage.
Embodiment 19. The oligomeric compound of embodiment 18, wherein each internucleoside linkage of the modified oligonucleotide is a modified internucleoside linkage.
Embodiment 20. The oligomeric compound of embodiment 18 or 19, wherein at least one internucleoside linkage is a phosphorothioate internucleoside linkage.
Embodiment 21. The oligomeric compound of embodiment 18 or 20, wherein the modified oligonucleotide comprises at least one phosphodiester internucleoside linkage.
Embodiment 22. The oligomeric compound of any of embodiments 18, 20, or 21, wherein each internucleoside linkage is independently selected from a phosphodiester internucleoside linkage or a phosphorothioate internucleoside linkage.
Embodiment 23. The oligomeric compound of any of embodiments 1-22, wherein the modified oligonucleotide comprises a modified nucleobase.
Embodiment 24. The oligomeric compound of embodiment 23, wherein the modified nucleobase is a 5-methyl cytosine.
Embodiment 25. The oligomeric compound of any of embodiments 1-24, wherein the modified oligonucleotide consists of 12-22, 12-20, 14-18, 14-20, 15-17, 15-25, 16-20, 16-18, 18-22 or 18-20 linked nucleosides.
Embodiment 26. The oligomeric compound of any of embodiments 1-25, wherein the modified oligonucleotide consists of 18 linked nucleosides.
Embodiment 27. The oligomeric compound of any of embodiments 1-25, wherein the modified oligonucleotide consists of 20 linked nucleosides.
Embodiment 28. The oligomeric compound of any of embodiments 1-27, consisting of the modified oligonucleotide.
Embodiment 29. The oligomeric compound of any of embodiments 1-27, comprising a conjugate group comprising a conjugate moiety and a conjugate linker.
Embodiment 30. The oligomeric compound of embodiment 29, wherein the conjugate linker consists of a single bond.
Embodiment 31. The oligomeric compound of embodiment 29, wherein the conjugate linker is cleavable.
Embodiment 32. The oligomeric compound of embodiment 29, wherein the conjugate linker comprises 1-3 linker-nucleosides.
Embodiment 33. The oligomeric compound of any of embodiments 29-32, wherein the conjugate group is attached to the modified oligonucleotide at the 5′-end of the modified oligonucleotide.
Embodiment 34. The oligomeric compound of any of embodiments 29-32, wherein the conjugate group is attached to the modified oligonucleotide at the 3′-end of the modified oligonucleotide.
Embodiment 35. The oligomeric compound of any of embodiments 1-27 and 29-34, comprising a terminal group.
Embodiment 36. The oligomeric compound of any of embodiments 1-35 wherein the oligomeric compound is a singled-stranded oligomeric compound.
Embodiment 37. The oligomeric compound of any of embodiments 1-31 or 33-36, wherein the oligomeric compound does not comprise linker-nucleosides.
Embodiment 38. An oligomeric duplex comprising an oligomeric compound of any of embodiments 1-27, 29-35, or 37.
Embodiment 39. An antisense compound comprising or consisting of an oligomeric compound of any of embodiments 1-37 or an oligomeric duplex of embodiment 38.
Embodiment 40. A pharmaceutical composition comprising an oligomeric compound of any of embodiments 1-37 or an oligomeric duplex of embodiment 38 and a pharmaceutically acceptable carrier or diluent.
Embodiment 41. The pharmaceutical composition of embodiment 40, wherein the pharmaceutically acceptable diluent is artificial cerebral spinal fluid, sterile saline, or PBS.
Embodiment 42. The pharmaceutical composition of embodiment 41, wherein the pharmaceutical composition consists essentially of the modified oligonucleotide and sterile saline.
Embodiment 43. A method comprising administering to an animal a pharmaceutical composition of any of embodiments 40-42.
Embodiment 44. A method of treating a disease associated with APP comprising administering to an individual having or at risk for developing a disease associated with APP a therapeutically effective amount of a pharmaceutical composition according to any of embodiments 40-42; and thereby treating the disease associated with APP.
Embodiment 45. The method of embodiment 44, wherein the APP-associated disease is Alzheimer's
Disease, Alzheimer's Disease in a Down Syndrome patient, or Cerebral Amyloid Angiopathy.
Embodiment 46. The method of any of embodiments 43-45, wherein at least one symptom or hallmark of the APP-associated disease is ameliorated.
Embodiment 47. The method of embodiment 46, wherein the symptom or hallmark is cognitive impairment, including a decline in memory and language skills, behavioral and psychological symptoms such as apathy and lack of motivation, gait disturbances and seizures, progressive dementia, and/or abnormal amyloid deposits.
Embodiment 48. An RNAi compound comprising an antisense RNAi oligonucleotide consisting of 17 to 30 linked nucleosides, wherein the nucleobase sequence of the antisense RNAi oligonucleotide comprises a targeting region comprising at least 15 contiguous nucleobases wherein the targeting region is at least 90% complementary to an equal length portion of an APP RNA, and wherein at least one nucleoside of the antisense RNAi oligonucleotide is a modified nucleoside comprising a modified sugar moiety or a sugar surrogate.
Embodiment 49. The RNAi compound of embodiment 48, wherein the antisense RNAi oligonucleotide consists of 18-25 linked nucleosides.
Embodiment 50. The RNAi compound of embodiment 48, wherein the antisense RNAi oligonucleotide consists of 20-25 linked nucleosides.
Embodiment 51. The RNAi compound of embodiment 48, wherein the antisense RNAi oligonucleotide consists of 21-23 linked nucleosides.
Embodiment 52. The RNAi compound of embodiment 48, wherein the antisense RNAi oligonucleotide consists of 21 linked nucleosides.
Embodiment 53. The RNAi compound of embodiment 48, wherein the antisense RNAi oligonucleotide consists of 23 linked nucleosides.
Embodiment 54. The RNAi compound of any of embodiments 48-53, wherein the targeting region of the antisense RNAi oligonucleotide is at least 95% complementary to the equal length portion of the APP RNA.
Embodiment 55. The RNAi compound of any of embodiments 48-53, wherein the targeting region of the antisense RNAi oligonucleotide is 100% complementary to the equal length portion of the APP RNA.
Embodiment 56. The RNAi compound of any of embodiments 48-55, wherein the targeting region of the antisense RNAi oligonucleotide comprises at least 19 contiguous nucleobases.
Embodiment 57. The RNAi compound of any of embodiments 48-55, wherein the targeting region of the antisense RNAi oligonucleotide comprises at least 21 contiguous nucleobases.
Embodiment 58. The RNAi compound of any of embodiments 48-55 wherein the targeting region of the antisense RNAi oligonucleotide comprises at least 25 contiguous nucleobases.
Embodiment 59. The RNAi compound of any embodiments 48-55, wherein the targeting region of the antisense RNAi oligonucleotide constitutes the entire nucleobase sequence of the antisense RNAi oligonucleotide.
Embodiment 60. The RNAi compound of any of embodiments 48-59 wherein the targeting region of the antisense oligonucleotide is complementary to an equal length portion of SEQ ID NOs: 1-7.
Embodiment 61. The RNAi compound of any of embodiments 48-60, wherein the APP RNA has the nucleobase sequence of any of SEQ ID NOs: 1-3 or SEQ ID NOs: 4-7.
Embodiment 62. The RNAi compound of any of embodiment 48-61, wherein the nucleobase sequence of the targeting region of the antisense RNAi compound is a least 12, 13, 14, 15, 16, 17, 18 19, 10, 21 nucleobases of any of SEQ ID NOs: 517-665, 815-840 or 867-888.
Embodiment 63. The RNAi compound of any of embodiments 48-62, wherein at least one nucleoside of the antisense RNAi oligonucleotide comprises a modified sugar moiety selected from: 2′-F, 2′-OMe, 2′-NMA, LNA, and cEt; or a sugar surrogate selected from GNA, and UNA.
Embodiment 64. The RNAi compound of any of embodiments 48-63, wherein each nucleoside of the antisense RNAi oligonucleotide comprises a modified sugar moiety or a sugar surrogate.
Embodiment 65. The compound of any of embodiments 48-64, wherein at least 80% of the nucleosides of the antisense RNAi oligonucleotide comprises a modified sugar moiety selected from 2′-F and 2′-OMe.
Embodiment 66. The RNAi compound of any of embodiments 65, wherein at least 90% of the nucleosides of the antisense RNAi oligonucleotide comprises a modified sugar moiety selected from 2′-F and 2′-OMe.
Embodiment 67. The RNAi compound of embodiment 66, wherein each nucleoside of the antisense RNAi oligonucleotide comprises a modified sugar moiety selected from 2′-F and 2′-OMe.
Embodiment 68. The RNAi compound of any of embodiments 48-67, wherein 1-4 nucleosides of the antisense RNAi oligonucleotide comprises a 2′-F modified sugar moiety.
Embodiment 69. The RNAi compound of embodiment 68, wherein at least 2 of the nucleosides of the antisense RNAi oligonucleotide comprising a 2′-F modified sugar moiety are adjacent to one another.
Embodiment 70. The RNAi compound of embodiment 69, wherein at least 3 nucleosides of the antisense RNAi oligonucleotide comprising a 2′-F modified sugar moiety are contiguous.
Embodiment 71. The RNAi compound of any of embodiments 48-66 or 68-70 wherein 1 nucleoside of the antisense RNAi oligonucleotide comprises GNA sugar surrogate.
Embodiment 72. The RNAi compound of embodiment 71, wherein the GNA sugar surrogate is (S)-GNA.
Embodiment 73. The RNAi compound of embodiment 71 or 72, wherein the nucleoside comprising the GNA sugar surrogate is at position 7 of the antisense RNAi oligonucleotide counting from the 5′-end.
Embodiment 74. The RNAi compound of any of embodiments 48-66 or 68-73 wherein 1 nucleoside of the antisense RNAi oligonucleotide is a UNA.
Embodiment 75. The RNAi compound of embodiment 74, wherein the nucleoside comprising the UNA sugar surrogate is at position 7 of the antisense RNAi oligonucleotide counting from the 5′-end.
Embodiment 76. The RNAi compound of any of embodiments 48-75, wherein at least one nucleoside of the antisense RNAi oligonucleotide comprises a modified nucleobase.
Embodiment 77. The RNAi compound of embodiment 76, wherein at least one nucleobase of the antisense RNAi oligonucleotide is inosine.
Embodiment 78. The RNAi compound of any of embodiments 48-77, wherein at least one internucleoside linkage of the antisense RNAi oligonucleotide is a modified internucleoside linkage.
Embodiment 79. The RNAi compound of embodiment 78, wherein at least one internucleoside linkage of the antisense RNAi oligonucleotide is a phosphorothioate internucleoside linkage.
Embodiment 80. The RNAi compound of any of embodiments 48-79, wherein each internucleoside linkage of the antisense RNAi oligonucleotide is selected from an unmodified phosphodiester internucleoside linkage and a phosphorothioate internucleoside linkage.
Embodiment 81. The RNAi compound of any of embodiments 79-80, wherein 1-3 internucleoside linkages at each end of the antisense RNAi oligonucleotide is a phosphorothioate internucleoside linkage.
Embodiment 82. The RNAi compound of embodiment 81, wherein 1-3 internucleoside linkages at each end of the antisense RNAi oligonucleotide is a phosphorothioate internucleoside linkage and all of the remaining internucleoside linkages of the antisense RNAi oligonucleotide are phosphodiester internucleoside linkages.
Embodiment 83. The RNAi compound of any of embodiments 48-82, comprising a sense RNAi oligonucleotide consisting of 17 to 30 linked nucleosides, wherein the nucleobase sequence of the sense RNAi oligonucleotide comprises an antisense-hybridizing region comprising least 15 contiguous nucleobases wherein the antisense-hybridizing region is at least 90% complementary to an equal length portion of the antisense RNAi oligonucleotide, wherein the sense RNAi oligonucleotide and the antisense RNAi oligonucleotide are hybridized to one another to form a duplex.
Embodiment 84. The RNAi compound of embodiment 83, wherein the sense RNAi oligonucleotide consists of 18-25 linked nucleosides.
Embodiment 85. The RNAi compound of embodiment 83, wherein the sense RNAi oligonucleotide consists of 20-25 linked nucleosides.
Embodiment 86. The RNAi compound of embodiment 83, wherein the sense RNAi oligonucleotide consists of 21-23 linked nucleosides.
Embodiment 87. The RNAi compound of embodiment 83, wherein the sense RNAi oligonucleotide consists of 21 linked nucleosides.
Embodiment 88. The RNAi compound of embodiment 83, wherein the sense RNAi oligonucleotide consists of 23 linked nucleosides.
Embodiment 89. The RNAi compound of any of embodiments 83-88, wherein the antisense-hybridizing region of the sense RNAi oligonucleotide is at least 95% complementary to the equal length portion of the antisense RNAi oligonucleotide.
Embodiment 90. The RNAi compound of any of embodiments 83-88, wherein the antisense-hybridizing region of the sense RNAi oligonucleotide is 100% complementary to the equal length portion of the antisense RNAi oligonucleotide.
Embodiment 91. The RNAi compound of any of embodiments 83-90, wherein the antisense-hybridizing region of the sense RNAi oligonucleotide comprises at least 20 contiguous nucleobases.
Embodiment 92. The RNAi compound of any of embodiments 83-90, wherein the antisense-hybridizing region of the sense RNAi oligonucleotide comprises at least 21 contiguous nucleobases.
Embodiment 93. The RNAi compound of any of embodiments 83-90, wherein the antisense-hybridizing region of the sense RNAi oligonucleotide comprises at least 25 contiguous nucleobases.
Embodiment 94. The RNAi compound of any embodiments 83-93, wherein the antisense-hybridizing region of the sense RNAi oligonucleotide constitutes the entire nucleobase sequence of the sense RNAi oligonucleotide.
Embodiment 95. The RNAi compound of any of embodiments 83-94, wherein 1-4 3′-most nucleosides of the antisense RNAi oligonucleotide are overhanging nucleosides.
Embodiment 96. The RNAi compound of any of embodiments 83-95, wherein 1-4 5′-most nucleosides of the antisense RNAi oligonucleotide are overhanging nucleosides.
Embodiment 97. The RNAi compound of any of embodiments 83-96, wherein 1-4 3′-most nucleosides of the sense RNAi oligonucleotide are overhanging nucleosides.
Embodiment 98. The RNAi compound of any of embodiments 83-97, wherein 1-4 4′-most nucleosides of the sense RNAi oligonucleotide are overhanging nucleosides.
Embodiment 99. The RNAi compound of any of embodiments 83-94, wherein the duplex is blunt ended at the 3′-end of the antisense RNAi oligonucleotide.
Embodiment 100. The RNAi compound of any of embodiments 83-94 or 99, wherein the duplex is blunt ended at the 5′-end of the antisense RNAi oligonucleotide.
Embodiment 101. The RNAi compound of any of embodiments 95-97, wherein at least one overhanging nucleoside is a deoxyribonucleoside.
Embodiment 102. The RNAi compound of any of embodiments 83-101, wherein at least one nucleoside of the sense RNAi oligonucleotide is a modified nucleoside.
Embodiment 103. The RNAi compound of embodiment 102, wherein at least one nucleoside of the sense RNAi oligonucleotide comprises a modified sugar moiety selected from: 2′-F, 2′-OMe, LNA, cEt, or a sugar surrogate selected from GNA, and UNA.
Embodiment 104. The RNAi compound of any of embodiments 83-103, wherein each nucleoside of the sense RNAi oligonucleotide comprises a modified sugar moiety or a sugar surrogate.
Embodiment 105. The RNAi compound of any of embodiments 83-104, wherein at least 80% of the nucleosides of the sense RNAi oligonucleotide comprises a modified sugar moiety selected from 2′-F and 2′-OMe.
Embodiment 106. The RNAi compound of embodiment 105, wherein each nucleoside of the sense RNAi oligonucleotide comprises a modified sugar moiety selected from 2′-F and 2′-OMe.
Embodiment 107. The RNAi compound of any of embodiments 83-106, wherein 1-4 nucleosides of the sense RNAi oligonucleotide comprises a 2′-F modified sugar moiety.
Embodiment 108. The RNAi compound of any of embodiments 83-107, wherein at least 2 nucleosides of the sense RNAi oligonucleotide comprising a 2′-F modified sugar moiety are adjacent to one another.
Embodiment 109. The RNAi compound of embodiment 108, wherein at least 3 nucleosides of the sense RNAi oligonucleotide comprising a 2′-F modified sugar moiety are contiguous.
Embodiment 110. The RNAi compound of any of embodiments 83-105 or 107-109 wherein at least one nucleoside of the sense RNAi oligonucleotide is a GNA.
Embodiment 111. The RNAi compound of any of embodiments 83-105 or 107-109 wherein one nucleoside of the sense RNAi oligonucleotide is a GNA.
Embodiment 112. The RNAi compound of embodiment 110 or 111, wherein the GNA sugar surrogate is (S)-GNA.
Embodiment 113. The RNAi compound of any of embodiments 83-105 or 107-109 wherein at least one nucleoside of the sense RNAi oligonucleotide is a UNA.
Embodiment 114. The RNAi compound of any of embodiments 83-105 or 107-109 wherein one nucleoside of the sense RNAi oligonucleotide is a UNA.
Embodiment 115. The RNAi compound of any of embodiments 83-114, wherein at least one nucleoside of the sense RNAi oligonucleotide comprises a modified nucleobase.
Embodiment 116. The RNAi compound of embodiment 115, wherein at least one nucleobase of the sense RNAi oligonucleotide is hypoxanthine.
Embodiment 117. The RNAi compound of any of embodiments 83-116, wherein at least one internucleoside linkage of the sense RNAi oligonucleotide is a modified internucleoside linkage.
Embodiment 118. The RNAi compound of embodiment 117, wherein at least one internucleoside linkage of the sense RNAi oligonucleotide is a phosphorothioate internucleoside linkage.
Embodiment 119. The RNAi compound of embodiment 118, wherein each internucleoside linkage of the sense RNAi oligonucleotide is selected from an unmodified phosphodiester internucleoside linkage and a phosphorothioate internucleoside linkage.
Embodiment 120. The RNAi compound of any of embodiments 117-119, wherein 1-3 internucleoside linkages at each end of the sense RNAi oligonucleotide is a phosphorothioate internucleoside linkage.
Embodiment 121. The RNAi compound of embodiment 120, wherein 1-3 internucleoside linkages at each end of the antisense RNAi oligonucleotide is a phosphorothioate internucleoside linkage and all of the remaining internucleoside linkages of the antisense RNAi oligonucleotide are phosphodiester internucleoside linkages.
Embodiment 122. The RNAi compound of any of embodiments 48-121 comprising a stabilized phosphate group attached to the 5′ position of the 5′-most nucleoside of the antisense RNAi oligonucleotide.
Embodiment 123. The RNAi compound of embodiment 122, wherein the stabilized phosphate group comprises a (E)-vinylphosphonate.
Embodiment 124. The RNAi compound of embodiment 122, wherein the stabilized phosphate group comprises a cyclopropyl phosphonate.
Embodiment 125. The RNAi compound of any of embodiments 48-124, wherein the compound comprises 1-5 abasic sugar moieties attached to one or both ends of the antisense RNA oligonucleotide.
Embodiment 126. The RNAi compound of embodiment 125, wherein the compound comprises one abasic sugar moiety attached to one or both ends of the antisense RNA oligonucleotide
Embodiment 127. The RNAi compound of embodiment 125 or 126, wherein each abasic sugar moiety is inverted.
Embodiment 128. The RNAi compound of any of embodiments 125-127, wherein the abasic sugar moieties are attached to the antisense RNA oligonucleotide through a phosphorothioate linkage.
Embodiment 129. The RNAi compound of any of embodiments 48-128, wherein the compound comprises 1-5 abasic sugar moieties attached to one or both ends of the sense RNA oligonucleotide.
Embodiment 130. The RNAi compound of embodiment 129, wherein the compound comprises one abasic sugar moiety attached to one or both ends of the sense RNA oligonucleotide
Embodiment 131. The RNAi compound of embodiment 129 or 130, wherein each abasic sugar moiety is inverted.
Embodiment 132. The RNAi compound of any of embodiments 129-131, wherein the abasic sugar moieties are attached to the sense RNA oligonucleotide through a phosphorothioate linkage.
Embodiment 133. The RNAi compound of any of embodiments 48-132, wherein the RNAi compound is a prodrug.
Embodiment 134. The RNAi compound of any of embodiments 48-132, wherein the compound comprises a conjugate group.
Embodiment 135. The RNAi compound of embodiment 134, wherein the conjugate group is conjugated to the antisense RNAi oligonucleotide.
Embodiment 136. The RNAi compound of embodiment 135, wherein the conjugate group is conjugated to the 5′-end of the antisense RNAi oligonucleotide.
Embodiment 137. The RNAi compound of embodiment 135, wherein the conjugate group is conjugated to the 3′-end of the antisense RNAi oligonucleotide.
Embodiment 138. The RNAi compound of embodiment 134, wherein the conjugate group is conjugated to the sense RNAi oligonucleotide.
Embodiment 139. The RNAi compound of embodiment 138, wherein the conjugate group is conjugated to the 5′-end of the sense RNAi oligonucleotide.
Embodiment 140. The RNAi compound of embodiment 138, wherein the conjugate group is conjugated to the 3′-end of the sense RNAi oligonucleotide.
Embodiment 141. The RNAi compound of any of embodiments 138-140, wherein the conjugate group is attached directly to the sense RNAi oligonucleotide.
Embodiment 142. The RNAi compound of any of embodiments 138-141, wherein the conjugate group is attached to the sense RNAi oligonucleotide through 1-5 abasic sugar moieties.
Embodiment 143. The RNAi compound of embodiment 142, wherein the 1-5 abasic sugar moieties are inverted.
Embodiment 144. The RNAi compound of any of embodiments 134-143, wherein the conjugate group comprises a pyrrolidine linker.
Embodiment 145. The RNAi compound of any of embodiments 134-144, wherein the conjugate group comprises a cell targeting moiety.
Embodiment 146. The RNAi compound of embodiment 145, wherein the cell targeting moiety is a neurotransmitter receptor ligand.
Embodiment 147. The RNAi compound of embodiment 146, wherein the targeting ligand targets a GABA transporter.
Embodiment 148. A pharmaceutical composition comprising the RNAi compound of any of embodiments 48-147 and a pharmaceutically acceptable carrier or diluent.
Embodiment 149. The pharmaceutical composition of embodiment 148, wherein the pharmaceutically acceptable diluent is artificial cerebral spinal fluid, sterile saline, or PBS.
Embodiment 150. The pharmaceutical composition of embodiment 149, wherein the pharmaceutical composition consists essentially of the RNAi compound and sterile saline.
Embodiment 151. The pharmaceutical composition of embodiment 148 or 149 comprising a lipid.
Embodiment 152. The pharmaceutical composition of embodiment 151 comprising a lipid nanoparticle.
Embodiment 153. A method comprising administering to an animal a pharmaceutical composition of any of embodiments 148-152.
Embodiment 154. A method of treating a disease associated with APP comprising administering to an individual having or at risk for developing a disease associated with APP a therapeutically effective amount of a pharmaceutical composition according to any of embodiments 148-152; and thereby treating the disease associated with APP.
Embodiment 155. The method of embodiment 154, wherein the APP-associated disease is Alzheimer's Disease, Alzheimer's Disease in a Down Syndrome patient, or Cerebral Amyloid Angiopathy.
Embodiment 156. The method of embodiment 155, wherein at least one symptom or hallmark of the APP-associated disease is ameliorated.
Embodiment 157. The method of embodiment 156, wherein the symptom or hallmark is cognitive impairment, including a decline in memory and language skills, behavioral and psychological symptoms such as apathy and lack of motivation, gait disturbances and seizures, progressive dementia, and/or abnormal amyloid deposits.
Embodiment 158. An oligomeric compound comprising a modified oligonucleotide consisting of 12 to 30 linked nucleosides wherein the nucleobase sequence of the modified oligonucleotide is at least 80% complementary to an equal length portion of an APP RNA, and wherein the modified oligonucleotide comprises at least one modification selected from a modified sugar, a sugar surrogate, and a modified internucleoside linkage.
Embodiment 159. An oligomeric compound comprising a modified oligonucleotide consisting of 12 to 30 linked nucleosides wherein the nucleobase sequence of the modified oligonucleotide comprises at least 12, 13, 14, 15, 16, 17, or 18 nucleobases of any of SEQ ID NOS: 12-501; wherein the modified oligonucleotide comprises at least one modification selected from a modified sugar and a modified internucleoside linkage.
Embodiment 160. An oligomeric compound comprising a modified oligonucleotide consisting of 12 to 30 linked nucleosides wherein the nucleobase sequence of the modified oligonucleotide comprises at least 12, 13, 14, 15, 16, 17, 18, 19, or 20 nucleobases of any of SEQ ID NOS: 502-516; wherein the modified oligonucleotide comprises at least one modification selected from a modified sugar and a modified internucleoside linkage.
Embodiment 161. An oligomeric compound comprising a modified oligonucleotide consisting of 12 to 30 linked nucleosides wherein the nucleobase sequence of the modified oligonucleotide comprises at least 12, 13, 14, 15, 16, 17, 18, 19, 20, 21, 22, or 23 nucleobases of any of SEQ ID NOS: 517-665, 815-840 or 867-888; wherein the modified oligonucleotide comprises at least one modification selected from a modified sugar and a modified internucleoside linkage.
Embodiment 162. An oligomeric compound comprising a modified oligonucleotide consisting of 12 to 30 linked nucleosides wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, or at least 21 contiguous nucleobases of:
   an equal length portion of nucleobases 40-78 of SEQ ID NO: 1;
   an equal length portion of nucleobases 69-146 of SEQ ID NO: 1;
   an equal length portion of nucleobases 83-129 of SEQ ID NO: 1;
   an equal length portion of nucleobases 83-246 of SEQ ID NO: 1;
   an equal length portion of nucleobases 94-225 of SEQ ID NO: 1;
   an equal length portion of nucleobases 194-231 of SEQ ID NO: 1;
   an equal length portion of nucleobases 194-238 of SEQ ID NO: 1;
   an equal length portion of nucleobases 236-268 of SEQ ID NO: 1;
   an equal length portion of nucleobases 258-288 of SEQ ID NO: 1;
   an equal length portion of nucleobases 285-311 of SEQ ID NO: 1;
   an equal length portion of nucleobases 296-321 of SEQ ID NO: 1;
   an equal length portion of nucleobases 307-330 of SEQ ID NO: 1;
   an equal length portion of nucleobases 329-352 of SEQ ID NO: 1;
   an equal length portion of nucleobases 330-352 of SEQ ID NO: 1;
   an equal length portion of nucleobases 339-383 of SEQ ID NO: 1;
   an equal length portion of nucleobases 415-439 of SEQ ID NO: 1;
   an equal length portion of nucleobases 413-477 of SEQ ID NO: 1;
   an equal length portion of nucleobases 415-477 of SEQ ID NO: 1;
   an equal length portion of nucleobases 477-506 of SEQ ID NO: 1;
   an equal length portion of nucleobases 477-523 of SEQ ID NO: 1;
   an equal length portion of nucleobases 477-541 of SEQ ID NO: 1;
   an equal length portion of nucleobases 530-557 of SEQ ID NO: 1;
   an equal length portion of nucleobases 581-638 of SEQ ID NO: 1;
   an equal length portion of nucleobases 636-661 of SEQ ID NO: 1;
   an equal length portion of nucleobases 652-697 of SEQ ID NO: 1;
   an equal length portion of nucleobases 728-821 of SEQ ID NO: 1;
   an equal length portion of nucleobases 770-821 of SEQ ID NO: 1;
   an equal length portion of nucleobases 920-950 of SEQ ID NO: 1;
   an equal length portion of nucleobases 1006-1049 of SEQ ID NO: 1;
   an equal length portion of nucleobases 1152-1179 of SEQ ID NO: 1;
   an equal length portion of nucleobases 1227-1265 of SEQ ID NO: 1;
   an equal length portion of nucleobases 1227-1274 of SEQ ID NO: 1;
   an equal length portion of nucleobases 1268-1332 of SEQ ID NO: 1;
   an equal length portion of nucleobases 1268-1311 of SEQ ID NO: 1;
   an equal length portion of nucleobases 1289-1332 of SEQ ID NO: 1;
   an equal length portion of nucleobases 1518-1543 of SEQ ID NO: 1;
   an equal length portion of nucleobases 1531-1593 of SEQ ID NO: 1;
   an equal length portion of nucleobases 1544-1593 of SEQ ID NO: 1;
   an equal length portion of nucleobases 1634-1657 of SEQ ID NO: 1;
   an equal length portion of nucleobases 1778-1800 of SEQ ID NO: 1;
   an equal length portion of nucleobases 1882-1908 of SEQ ID NO: 1;
   an equal length portion of nucleobases 2051-2074 of SEQ ID NO: 1;
   an equal length portion of nucleobases 2360-3117 of SEQ ID NO: 1;
   an equal length portion of nucleobases 2402-3117 of SEQ ID NO: 1;
   an equal length portion of nucleobases 2360-2655 of SEQ ID NO: 1;
   an equal length portion of nucleobases 2402-2655 of SEQ ID NO: 1;
   an equal length portion of nucleobases 2675-3054 of SEQ ID NO: 1; or
   an equal length portion of nucleobases 3192-3277 of SEQ ID NO: 3; wherein the modified oligonucleotide comprises at least one modification selected from a modified sugar and a modified internucleoside linkage.
Embodiment 163. The oligomeric compound of any of embodiments 158-162, wherein the modified oligonucleotide has a nucleobase sequence that is at least 80%, 85%, 90%, 95%, or 100% complementary to any of the nucleobase sequences of SEQ ID NO: 1-7 when measured across the entire nucleobase sequence of the modified oligonucleotide.
Embodiment 164. The oligomeric compound of any of embodiments 158-162, wherein at least one nucleoside of the modified oligonucleotide is a modified nucleoside.
Embodiment 165. The oligomeric compound of embodiment 164, wherein at least one modified nucleoside of the modified oligonucleotide comprises a modified sugar moiety.
Embodiment 166. The oligomeric compound of embodiment 165, wherein at least one modified nucleoside of the modified oligonucleotide comprises a bicyclic sugar moiety.
Embodiment 167. The oligomeric compound of embodiment 166, wherein at least one modified nucleoside of the modified oligonucleotide comprises a bicyclic sugar moiety having a 2′-4′ bridge, wherein the 2′-4′ bridge is selected from —O—CH₂—; and —O—CH(CH₃)—.
Embodiment 168. The oligomeric compound of any of embodiments 162-167, wherein at least one modified nucleoside of the modified oligonucleotide comprises a non-bicyclic modified sugar moiety.
Embodiment 169. The oligomeric compound of embodiment 168, wherein at least one modified nucleoside of the modified oligonucleotide comprises a bicyclic sugar moiety having a 2′-4′ bridge and at least one nucleoside of the modified oligonucleotide comprises a non-bicyclic modified sugar moiety.
Embodiment 170. The oligomeric compound of embodiment 168 or 169, wherein the non-bicyclic modified sugar moiety is a 2′-MOE modified sugar moiety, a 2′-OMe modified sugar moiety, or a 2′-F modified sugar moiety.
Embodiment 171. The oligomeric compound of any of embodiments 158-170, wherein tat least one modified nucleoside of the modified oligonucleotide comprises a sugar surrogate.
Embodiment 172. The oligomeric compound of embodiment 171, wherein the modified oligonucleotide comprises at least one modified nucleoside comprising a sugar surrogate selected from morpholino and PNA.
Embodiment 173. The oligomeric compound of any of embodiments 158-172, wherein the modified oligonucleotide comprises at least one modified internucleoside linkage.
Embodiment 174. The oligomeric compound of embodiment 173, wherein each internucleoside linkage of the modified oligonucleotide is a modified internucleoside linkage.
Embodiment 175. The oligomeric compound of embodiment 173 or 174, wherein at least one internucleoside linkage is a phosphorothioate internucleoside linkage.
Embodiment 176. The oligomeric compound of embodiment 173 or 175, wherein the modified oligonucleotide comprises at least one phosphodiester internucleoside linkage.
Embodiment 177. The oligomeric compound of any of embodiments 173, 175, or 176, wherein each internucleoside linkage is independently selected from a phosphodiester internucleoside linkage or a phosphorothioate internucleoside linkage.
Embodiment 178. The oligomeric compound of any of embodiments 158-177, wherein the modified oligonucleotide comprises a modified nucleobase.
Embodiment 179. The oligomeric compound of embodiment 178, wherein the modified nucleobase is a 5-methyl cytosine.
Embodiment 180. The oligomeric compound of any of embodiments 158-179 wherein the modified oligonucleotide consists of 12-22, 12-20, 14-18, 14-20, 15-17, 15-25, 16-20, 16-18, 18-22, 18-25, 18-20, 20-25, or 21-23 linked nucleosides.
Embodiment 181. The oligomeric compound of any of embodiments 158-180, wherein the modified oligonucleotide consists of 18 linked nucleosides.
Embodiment 182. The oligomeric compound of any of embodiments 158-180, wherein the modified oligonucleotide consists of 20 linked nucleosides.
Embodiment 183. The oligomeric compound of any of embodiments 158-180, wherein the modified oligonucleotide consists of 21 linked nucleosides.
Embodiment 184. The oligomeric compound of any of embodiments 158-180, wherein the modified oligonucleotide consists of 23 linked nucleosides.
Embodiment 185. The oligomeric compound of any of embodiments 158-184, wherein the oligomeric compound is an RNase H compound.
Embodiment 186. The oligomeric compound of embodiment 185, wherein the modified oligonucleotide is a gapmer.
Embodiment 187. The oligomeric compound of any of claims 158-186, wherein the modified oligonucleotide has a sugar motif comprising:
   a 5′-region consisting of 1-6 linked 5′-region nucleosides;
   a central region consisting of 6-10 linked central region nucleosides; and
   a 3′-region consisting of 1-6 linked 3′-region nucleosides;
   wherein the 3′-most nucleoside of the 5′-region and the 5′-most nucleoside of the 3′-region comprise modified sugar moieties, and
   each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprising a 2′-substituted sugar moiety.
Embodiment 188. The oligomeric compound of any of embodiments 158-183 or 185-187, wherein the modified oligonucleotide has a sugar motif comprising:
   a 5′-region consisting of 1-6 linked 5′-region nucleosides;
   a central region consisting of 6-10 linked central region nucleosides; and
   a 3′-region consisting of 1-6 linked 3′-region nucleosides; wherein
   each of the 5′-region nucleosides and each of the 3′-region nucleosides comprises a modified sugar moiety and each of the central region nucleosides comprises a 2′-β-D-deoxyribosyl sugar moiety.
Embodiment 189. The oligomeric compound of embodiment 188, wherein the modified oligonucleotide has a sugar motif comprising:
   a 5′-region consisting of 5 linked 5′-region nucleosides;
   a central region consisting of 10 linked central region nucleosides; and
   a 3′-region consisting of 5 linked 3′-region nucleosides; wherein
   each of the 5′-region nucleosides and each of the 3′-region nucleosides comprises either a cEt modified sugar moiety or a 2′-MOE modified sugar moiety, and each of the central region nucleosides comprises a 2′-β-D-deoxyribosyl sugar moiety.
Embodiment 190. The oligomeric compound of any of embodiments 158-184, wherein the oligomeric compound is an RNAi compound.
Embodiment 191. The oligomeric compound of any of embodiments 158-190, wherein the oligomeric compound comprises an antisense RNAi oligonucleotide comprising a targeting region comprising at least 15 contiguous nucleobases, wherein the targeting region is at least 90% complementary to an equal-length portion of an APP RNA.
Embodiment 192. The oligomeric compound of embodiment 191, wherein the targeting region of the antisense RNAi oligonucleotide is at least 95% complementary or is 100% complementary to the equal length portion of an APP RNA.
Embodiment 193. The oligomeric compound of any of embodiments 191-192, wherein the targeting region of the antisense RNAi oligonucleotide comprises at least 19, 20, 21, or 25 contiguous nucleobases.
Embodiment 194. The oligomeric compound of any of embodiments 191-193, wherein the APP RNA has the nucleobase sequence of any of SEQ ID NOs: 1-7.
Embodiment 195. The oligomeric compound of any of embodiments 191-194 wherein at least one nucleoside of the antisense RNAi oligonucleotide comprises a modified sugar moiety selected from: 2′-F, 2′-OMe, 2′-NMA, LNA, and cEt; or a sugar surrogate selected from GNA, and UNA.
Embodiment 196. The oligomeric compound of any of embodiments 191-195, wherein each nucleoside of the antisense RNAi oligonucleotide comprises a modified sugar moiety or a sugar surrogate.
Embodiment 197. The oligomeric compound of any of embodiments 191-196 wherein at least 80%, at least 90%, or 100% of the nucleosides of the antisense RNAi oligonucleotide comprises a modified sugar moiety selected from 2′-F and 2′-OMe.
Embodiment 198. The oligomeric compound of any of embodiments 191-197, comprising a stabilized phosphate group attached to the 5′ position of the 5′-most nucleoside of the antisense RNAi oligonucleotide.
Embodiment 199. The oligomeric compound of embodiment 198, wherein the stabilized phosphate group comprises a cyclopropyl phosphonate or an (E)-vinyl phosphonate.
Embodiment 200. The oligomeric compound of any of embodiments 158-199, wherein the oligomeric compound is a single-stranded oligomeric compound.
Embodiment 201. The oligomeric compound of any of embodiments 158-200, consisting of the modified oligonucleotide or the RNAi antisense oligonucleotide.
Embodiment 202. The oligomeric compound of any of embodiments 158-200 comprising a conjugate group comprising a conjugate moiety and a conjugate linker.
Embodiment 203. The oligomeric compound of embodiment 202, wherein the conjugate linker consists of a single bond.
Embodiment 204. The oligomeric compound of embodiment 202, wherein the conjugate linker is cleavable.
Embodiment 205. The oligomeric compound of embodiment 202, wherein the conjugate linker comprises 1-3 linker-nucleosides.
Embodiment 206. The oligomeric compound of any of embodiments 202-205, wherein the conjugate group is attached to the 5′-end of the modified oligonucleotide or the antisense RNAi oligonucleotide.
Embodiment 207. The oligomeric compound of any of embodiments 202-205 wherein the conjugate group is attached to the 3′-end of the modified oligonucleotide or the antisense RNAi oligonucleotide.
Embodiment 208. The oligomeric compound of any of embodiments 158-200 or 202-206, comprising a terminal group.
Embodiment 209. The oligomeric compound of any of embodiments 158-204 or 206-208, wherein the oligomeric compound does not comprise linker-nucleosides.
Embodiment 210. An oligomeric duplex, comprising a first oligomeric compound comprising an antisense RNAi oligonucleotide of any of embodiments 188-209 and a second oligomeric compound comprising a sense RNAi oligonucleotide consisting of 17 to 30 linked nucleosides, wherein the nucleobase sequence of the sense RNAi oligonucleotide comprises an antisense-hybridizing region comprising least 15 contiguous nucleobases wherein the antisense-hybridizing region is at least 90% complementary to an equal length portion of the antisense RNAi oligonucleotide.
Embodiment 211. The oligomeric duplex of embodiment 210, wherein the sense RNAi oligonucleotide consists of 18-25, 20-25, or 21-23 linked nucleosides.
Embodiment 212. The oligomeric duplex of embodiment 211, wherein the sense RNAi oligonucleotide consists of 21 or 23 linked nucleosides.
Embodiment 213. The oligomeric duplex of any of embodiments 210-212, wherein 1-4 3′-most nucleosides of the antisense or the sense RNAi oligonucleotide are overhanging nucleosides.
Embodiment 214. The oligomeric duplex of any of embodiments 210-213, wherein 1-4 5′-most nucleosides of the antisense or sense RNAi oligonucleotide are overhanging nucleosides.
Embodiment 215. The oligomeric duplex of any of embodiments 210-214, wherein the duplex is blunt ended at the 3′-end of the antisense RNAi oligonucleotide.
Embodiment 216. The oligomeric duplex of any of embodiments 210-214, wherein the duplex is blunt ended at the 5′-end of the antisense RNAi oligonucleotide.
Embodiment 217. The oligomeric duplex of any of embodiments 210-216, wherein at least one nucleoside of the sense RNAi oligonucleotide comprises a modified sugar moiety selected from: 2′-F, 2′-OMe, LNA, cEt, or a sugar surrogate selected from GNA, and UNA.
Embodiment 218. The oligomeric duplex of embodiment 217, wherein each nucleoside of the sense RNAi oligonucleotide comprises a modified sugar moiety or a sugar surrogate.
Embodiment 219. The oligomeric duplex of embodiment 218, wherein at least 80%, at least 90%, or 100% of the nucleosides of the sense RNAi oligonucleotide comprises a modified sugar moiety selected from 2′-F and 2′-OMe.
Embodiment 220. The oligomeric duplex of any of embodiments 210-219, wherein at least one nucleoside of the sense RNAi oligonucleotide comprises a modified nucleobase.
Embodiment 221. The oligomeric duplex of any of embodiments 210-220, wherein at least one internucleoside linkage of the sense RNAi oligonucleotide is a modified internucleoside linkage.
Embodiment 222. The oligomeric duplex of embodiment 221, wherein at least one internucleoside linkage of the sense RNAi oligonucleotide is a phosphorothioate internucleoside linkage.
Embodiment 223. The oligomeric duplex of any of embodiments 210-222, wherein the compound comprises 1-5 abasic sugar moieties attached to one or both ends of the antisense or sense RNA oligonucleotide.
Embodiment 224. The oligomeric duplex of any of embodiments 210-223, consisting of the antisense RNAi oligonucleotide and the sense RNAi oligonucleotide.
Embodiment 225. The oligomeric duplex of embodiment 210, wherein the second oligomeric compound comprises a conjugate group comprising a conjugate moiety and a conjugate linker.
Embodiment 226. The oligomeric duplex of embodiment 225, wherein the conjugate linker consists of a single bond.
Embodiment 227. The oligomeric duplex of embodiment 225, wherein the conjugate linker is cleavable.
Embodiment 228. The oligomeric duplex of embodiment 225, wherein the conjugate linker comprises 1-3 linker-nucleosides.
Embodiment 229. The oligomeric duplex of any of embodiments 225-228, wherein the conjugate group is attached to the 5′-end of the sense RNAi oligonucleotide.
Embodiment 230. The oligomeric compound of any of embodiments 225-225 wherein the conjugate group is attached to the 3′-end of the sense RNAi oligonucleotide.
Embodiment 231. The oligomeric compound of any of embodiments 225-225 wherein the conjugate group is attached via the 2′ position of a ribosyl sugar moiety at an internal position within the sense RNAi oligonucleotide.
Embodiment 232. The oligomeric compound of any of embodiments 202-207 or the oligomeric duplex of any of embodiments 225-231, wherein at least one conjugate group comprises a C₁₆ alkyl group.
Embodiment 233. The oligomeric duplex of embodiment 210, wherein the second oligomeric compound comprises a terminal group.
Embodiment 234. A pharmaceutical composition comprising an oligomeric compound of any of embodiments 158-209 or an oligomeric duplex of embodiments 210-233 and a pharmaceutically acceptable carrier or diluent.
Embodiment 235. The pharmaceutical composition of embodiment 234, wherein the pharmaceutically acceptable diluent is artificial cerebral spinal fluid, sterile saline, or PBS.
Embodiment 236. The pharmaceutical composition of embodiment 234, wherein the pharmaceutical composition consists essentially of the modified oligonucleotide and sterile saline.
Embodiment 237. A method comprising administering to an animal a pharmaceutical composition of any of embodiments 234-236.
Embodiment 238. A method of treating a disease associated with APP comprising administering to an individual having or at risk for developing a disease associated with APP a therapeutically effective amount of a pharmaceutical composition according to any of embodiments 234-236; and thereby treating the disease associated with APP.
Embodiment 239. The method of embodiment 238, wherein the APP-associated disease is Alzheimer's Disease, Alzheimer's Disease in a Down Syndrome patient, or Cerebral Amyloid Angiopathy.
Embodiment 240. The method of any of embodiments 238-239 wherein at least one symptom or hallmark of the APP-associated disease is ameliorated.
Embodiment 241. The method of embodiment 240, wherein the symptom or hallmark is cognitive impairment, including a decline in memory and language skills, behavioral and psychological symptoms such as apathy and lack of motivation, gait disturbances and seizures, progressive dementia, and/or abnormal amyloid deposits.

I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. In certain embodiments, provided herein are RNAi compounds comprising antisense RNAi oligonucleotides complementary to APP and optionally sense RNAi oligonucleotides complementary to the antisense RNAi oligonucleotides. Antisense RNAi oligonucleotides and sense RNAi oligonucleotides typically comprise at least one modified nucleoside and/or at least one modified internucleoside linkage. Certain modified nucleosides and modified internucleoside linkages suitable for use in modified oligonucleotides are described below.

A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into antisense RNAi oligonucleotides and/or sense RNAi oligonucleotides.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 3′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃(“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), 2′-OCH₂OCH₂N(CH₂)₂ (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. In certain embodiments, non-bicyclic modified sugar moieties comprise a substituent group at the 3′-position. Examples of substituent groups suitable for the 3′-position of modified sugar moieties include but are not limited to alkoxy (e.g., methoxy), alkyl (e.g., methyl, ethyl). In certain embodiments, non-bicyclic modified sugar moieties comprise a substituent group at the 4′-position. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, ethyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In certain embodiments, a 2′-substituted non-bicyclic modified nucleoside comprises a sugar moiety comprising a non-bridging 2′-substituent group selected from: F, NH₂, N3, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(124)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)2, O(CH₂)₂O(CH₂)₂N(CH₃)2, O(CH₂)₂ON(CH₃)2 (“DMAOE”), OCH₂OCH₂N(CH₂)₂ (“DMAEOE”) and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

In naturally occurring nucleic acids, sugars are linked to one another 3′ to 5′. In certain embodiments, oligonucleotides include one or more nucleoside or sugar moiety linked at an alternative position, for example at the 2′ or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. n certain such embodiments, the furanose ring is a ribose ring. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′- (CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Ra)(Rb)]n-, —[C(Ra)(Rb)]n-O—, C(Ra)═C(Rb)-, C(Ra)═N—, C(═NRa)-, —C(═O)—, —C(═S)—, —O—, —Si(Ra)2-, —S(═O)x-, and N(Ra)-;

wherein:

x is 0, 1, or 2;

n is 1, 2, 3, or 4;

each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1 acyl (C(═O)—H), substituted acyl, CN, sulfonyl (S(═O)2-J1), or sulfoxyl (S(═O)-J1); and each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727. In certain embodiments, bicyclic sugar moieties and nucleosides incorporating such bicyclic sugar moieties are further defined by isomeric configuration. For example, an LNA nucleoside (described herein) may be in the α-L configuration or in the 0-D configuration.

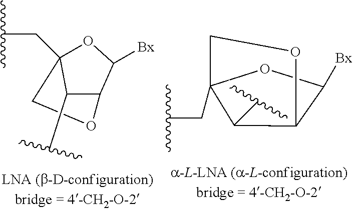

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mal Cane Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

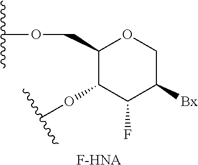

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

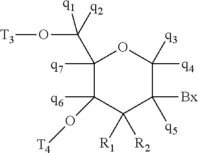

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T3 and T4 is an internucleoside linking group linking the modified THP nucleoside to the remainder of an oligonucleotide and the other of T3 and T4 is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

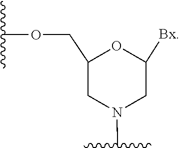

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the RNAi oligonucleotides of the invention are described in, for example, in Nielsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

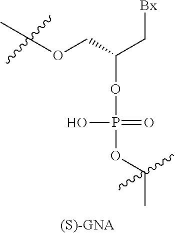

where Bx represents any nucleobase.

Many other bicyclic and tricyclic sugar and sugar surrogats are known in the art that can be used in modified nucleosides.

2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 5-methylcytosine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

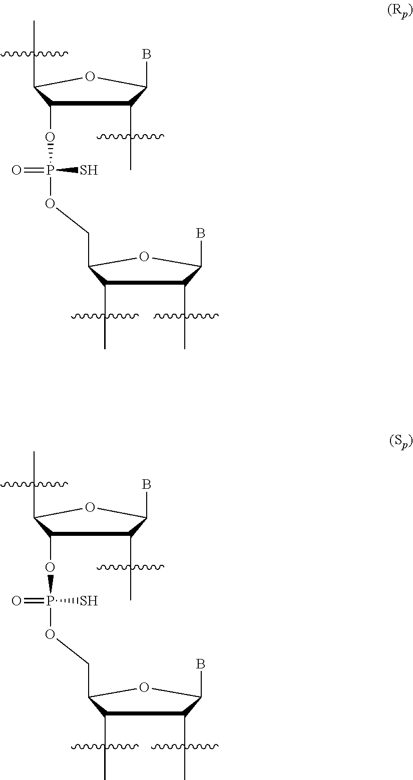

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′—CH₂—N(CH₃)—O-5), amide-3 (3′—CH₂—C(═O)—N(H)-5), amide-4 (3′—CH₂—N(H)—C(═O)-5), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides (such as antisense RNAi oligonucleotides and/or sense RNAi oligonucleotides) comprise one or more inverted nucleoside, as shown below:

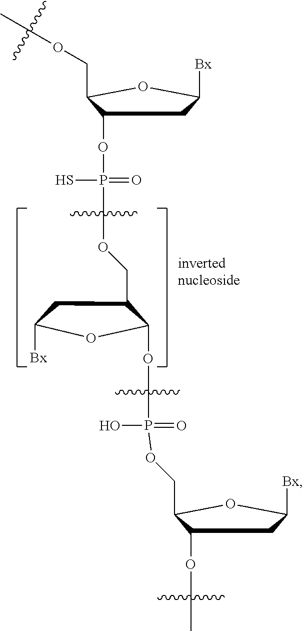

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

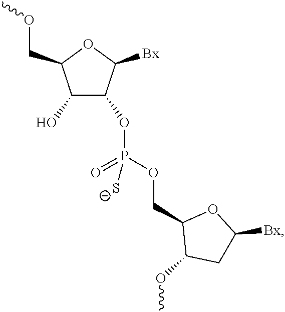

wherein each Bx represents any nucleobase.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

### Uniformly Modified Oligonucleotides

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified nucleotide comprises the same 2′-modification.

### Gapmer Oligonucleotides

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, at least one nucleoside of the gap of a gapmer comprises a 2′-OMe sugar moiety.

Herein, the lengths (number of nucleosides) of the three regions of a gapmer may be provided using the notation [# of nucleosides in the 5′-wing]−[# of nucleosides in the gap]−[# of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise 2′-β-D-deoxyribosyl sugar moieties. Thus, a 5-10-5 MOE gapmer consists of 5 linked 2′-MOE nucleosides in the 5′-wing, 10 linked 2′-β-D-deoxynucleosides in the gap, and 5 linked 2′-MOE nucleosides in the 3′-wing. A 3-10-3 cEt gapmer consists of 3 linked cEt nucleosides in the 5′-wing, 10 linked 2′-α-D-deoxynucleosides in the gap, and 3 linked cEt nucleosides in the 3′-wing. A 5-8-5 gapmer consists of 5 linked nucleosides comprising a modified sugar moiety in the 5′-wing, 8 linked 2′-β-D-deoxynucleosides in the gap, and 5 linked nucleosides comprising a modified sugar moiety in the 3′-wing. A 5-8-5 mixed gapmer has at least two different modified sugar moieties in the 5′- and/or the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

In certain embodiments, modified oligonucleotides are 5-8-5 mixed gapmers that consist of 5 linked 2′-MOE nucleosides in the 5′-wing, 8 linked 2′-β-D-deoxynucleosides in the gap, and a mixture of cEt and 2′-MOE nucleosides in the 3′-wing. In certain embodiments, modified nucleosides have a sugar motif of eeeeeddddddddkkeee, where each “e” represents a nucleoside comprising a 2′-MOE modified sugar moiety, each “d” represents a nucleoside comprising a 2′-β-D-deoxyribosyl sugar moiety, and each “k” represents a nucleoside comprising a cEt modified sugar moiety. In certain embodiments, modified nucleosides have a sugar motif of eeeeeddddddddkeeee, where each “e” represents a nucleoside comprising a 2′-MOE modified sugar moiety, each “d” represents a nucleoside comprising a 2′-β-D-deoxyribosyl sugar moiety, and each “k” represents a nucleoside comprising a cEt modified sugar moiety.

### Antisense RNAi Oligonucleotides

In certain embodiments, the sugar moiety of at least one nucleoside of an antisense RNAi oligonucleotide is a modified sugar moiety.

In certain such embodiments, at least one nucleoside comprises a 2′-OMe modified sugar moiety. In certain embodiments, at least 2 nucleosides comprise 2′-OMe modified sugar moieties. In certain embodiments, at least 5 nucleosides comprise 2′-OMe modified sugar moieties. In certain embodiments, at least 8 nucleosides comprise 2′-OMe modified sugar moieties. In certain embodiments, at least 10 nucleosides comprise 2′-OMe modified sugar moieties. In certain embodiments, at least 12 nucleosides comprise 2′-OMe modified sugar moieties. In certain embodiments, at least 14 nucleosides comprise 2′-OMe modified sugar moieties. In certain embodiments, at least 15 nucleosides comprise 2′-OMe modified sugar moieties. In certain embodiments, at least 17 nucleosides comprise 2′-OMe modified sugar moieties. In certain such embodiments, at least 18 nucleosides comprise 2′-OMe modified sugar moieties. In certain such embodiments, at least 20 nucleosides comprise 2′-OMe modified sugar moieties. In certain such embodiments, at least 21 nucleosides comprise 2′-OMe modified sugar moieties.

In certain embodiments, at least one nucleoside comprises a 2′-F modified sugar. In certain embodiments, at least 2 nucleosides comprise 2′-F modified sugar moieties. In certain embodiments, at least 3 nucleosides comprise 2′-F modified sugar moieties. In certain embodiments, at least 4 nucleosides comprise 2′-F modified sugar moieties. In certain embodiments, one, but not more than one nucleoside comprises a 2′-F modified sugar. In certain embodiments, 1 or 2 nucleosides comprise 2′-F modified sugar moieties. In certain embodiments, 1-3 nucleosides comprise 2′-F modified sugar moieties. In certain embodiments, at least 1-4 nucleosides comprise 2′-F modified sugar moieties. In certain embodiments, antisense RNAi oligonucleotides have a block of 2-4 contiguous 2′-F modified nucleosides. In certain embodiments, 4 nucleosides of an antisense RNAi oligonucleotide are 2′-F modified nucleosides and 3 of those 2′-F modified nucleosides are contiguous. In certain such embodiments the remainder of the nucleosides are 2′OMe modified.

### Sense RNAi Oligonucleotides

In certain embodiments, the sugar moiety of at least one nucleoside of a sense RNAi oligonucleotides is a modified sugar moiety.

In certain such embodiments, at least one nucleoside comprises a 2′-OMe modified sugar moiety. In certain embodiments, at least 2 nucleosides comprise 2′-OMe modified sugar moieties. In certain embodiments, at least 5 nucleosides comprise 2′-OMe modified sugar moieties. In certain embodiments, at least 8 nucleosides comprise 2′-OMe modified sugar moieties. In certain embodiments, at least 10 nucleosides comprise 2′-OMe modified sugar moieties. In certain embodiments, at least 12 nucleosides comprise 2′-OMe modified sugar moieties. In certain embodiments, at least 14 nucleosides comprise 2′-OMe modified sugar moieties. In certain embodiments, at least 15 nucleosides comprise 2′-OMe modified sugar moieties. In certain embodiments, at least 17 nucleosides comprise 2′-OMe modified sugar moieties. In certain such embodiments, at least 18 nucleosides comprise 2′-OMe modified sugar moieties. In certain such embodiments, at least 20 nucleosides comprise 2′-OMe modified sugar moieties. In certain such embodiments, at least 21 nucleosides comprise 2′-OMe modified sugar moieties.

In certain embodiments, at least one nucleoside comprises a 2′-F modified sugar moiety. In certain embodiments, at least 2 nucleosides comprise 2′-F modified sugar moieties. In certain embodiments, at least 3 nucleosides comprise 2′-F modified sugar moieties. In certain embodiments, at least 4 nucleosides comprise 2′-F modified sugar moieties. In certain embodiments, one, but not more than nucleoside comprises a 2′-F modified sugar moiety. In certain embodiments, 1 or 2 nucleosides comprise 2′-F modified sugar moieties. In certain embodiments, 1-3 nucleosides comprise 2′-F modified sugar moieties. In certain embodiments, at least 1-4 nucleosides comprise 2′-F modified sugar moieties. In certain embodiments, sense RNAi oligonucleotides have a block of 2-4 contiguous 2′-F modified nucleosides. In certain embodiments, 4 nucleosides of a sense RNAi oligonucleotide are 2′-F modified nucleosides and 3 of those 2′-F modified nucleosides are contiguous. In certain such embodiments the remainder of the nucleosides are 2′OMe modified.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

### Gapmer Oligonucleotides

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety. In certain embodiments, the modified nucleobase is selected from: a 2-thiopyrimidine and a 5-propynepyrimidine.

### Antisense RNAi Oligonucleotides

In certain embodiments, one nucleoside of an antisense RNAi oligonucleotide is a UNA. In certain embodiments, one nucleoside of an antisense RNAi oligonucleotide is a GNA. In certain embodiments, 1-4 nucleosides of an antisense RNAi oligonucleotide is/are DNA. In certain such embodiments, the 1-4 DNA nucleosides are at one or both ends of the antisense RNAi oligonucleotide.

### Sense RNAi Oligonucleotides

In certain embodiments, one nucleoside of a sense RNAi oligonucleotide is a UNA.

In certain embodiments, one nucleoside of a sense RNAi oligonucleotide is a GNA.

In certain embodiments, 1-4 nucleosides of a sense RNAi oligonucleotide is/are DNA. In certain such embodiments, the 1-4 DNA nucleosides are at one or both ends of the sense RNAi oligonucleotide.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (β=S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate.

### Gapmer Oligonucleotides

In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, modified nucleotides have an internucleoside linkage motif of sososssssssssosss, wherein each “s” represents a phosphorothioate internucleoside linkage and each “o” represents a phosphate internucleoside linkage. In certain embodiments, modified nucleotides have an internucleoside linkage motif of sooosssssssssooss, wherein each “s” represents a phosphorothioate internucleoside linkage and each “o” represents a phosphate internucleoside linkage. In certain embodiments, modified nucleotides have an internucleoside linkage motif of sooosssssssssooss, wherein each “s” represents a phosphorothioate internucleoside linkage and each “o” represents a phosphate internucleoside linkage.

### Antisense RNAi Oligonucleotides

In certain embodiments, at least one linkage of the antisense RNAi oligonucleotide is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain such embodiments, the modified linkage is a phosphorothioate linkage. In certain embodiments, the remaining linkages are all unmodified phosphodiester linkages.

In certain embodiments, at least one linkage of the antisense RNAi oligonucleotide is an inverted linkage.

### Sense RNAi Oligonucleotides

In certain embodiments, at least one linkage of the sense RNAi oligonucleotides is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain such embodiments, the modified linkage is a phosphorothioate linkage. In certain embodiments, the remaining linkages are all unmodified phosphodiester linkages.

In certain embodiments, at least one linkage of the sense RNAi oligonucleotides is an inverted linkage.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### Antisense RNAi Oligonucleotides

In certain embodiments, antisense RNAi oligonucleotides consist of 17-30 linked nucleosides. In certain embodiments, antisense RNAi oligonucleotides consist of 17-25 linked nucleosides. In certain embodiments, antisense RNAi oligonucleotides consist of 17-23 linked nucleosides. In certain embodiments, antisense RNAi oligonucleotides consist of 17-21 linked nucleosides. In certain embodiments, antisense RNAi oligonucleotides consist of 18-30 linked nucleosides. In certain embodiments, antisense RNAi oligonucleotides consist of 20-30 linked nucleosides. In certain embodiments, antisense RNAi oligonucleotides consist of 21-30 linked nucleosides. In certain embodiments, antisense RNAi oligonucleotides consist of 23-30 linked nucleosides. In certain embodiments, antisense RNAi oligonucleotides consist of 18-25 linked nucleosides. In certain embodiments, antisense RNAi oligonucleotides consist of 20-22 linked nucleosides. In certain embodiments, antisense RNAi oligonucleotides consist of 21-23 linked nucleosides. In certain embodiments, antisense RNAi oligonucleotides consist of 23-24 linked nucleosides. In certain embodiments, antisense RNAi oligonucleotides consist of 20 linked nucleosides. In certain embodiments, antisense RNAi oligonucleotides consist of 21 linked nucleosides. In certain embodiments, antisense RNAi oligonucleotides consist of 22 linked nucleosides. In certain embodiments, antisense RNAi oligonucleotides consist of 23 linked nucleosides.

### Sense RNAi Oligonucleotides

In certain embodiments, sense RNAi oligonucleotides consist of 17-30 linked nucleosides. In certain embodiments, sense RNAi oligonucleotides consist of 17-25 linked nucleosides. In certain embodiments, sense RNAi oligonucleotides consist of 17-23 linked nucleosides. In certain embodiments, sense RNAi oligonucleotides consist of 17-21 linked nucleosides. In certain embodiments, sense RNAi oligonucleotides consist of 18-30 linked nucleosides. In certain embodiments, sense RNAi oligonucleotides consist of 20-30 linked nucleosides. In certain embodiments, sense RNAi oligonucleotides consist of 21-30 linked nucleosides. In certain embodiments, sense RNAi oligonucleotides consist of 23-30 linked nucleosides. In certain embodiments, sense RNAi oligonucleotides consist of 18-25 linked nucleosides. In certain embodiments, sense RNAi oligonucleotides consist of 20-22 linked nucleosides. In certain embodiments, sense RNAi oligonucleotides consist of 21-23 linked nucleosides. In certain embodiments, sense RNAi oligonucleotides consist of 23-24 linked nucleosides. In certain embodiments, sense RNAi oligonucleotides consist of 20 linked nucleosides. In certain embodiments, sense RNAi oligonucleotides consist of 21 linked nucleosides. In certain embodiments, sense RNAi oligonucleotides consist of 22 linked nucleosides. In certain embodiments, sense RNAi oligonucleotides consist of 23 linked nucleosides.

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain RNAi Compounds

RNAi compounds comprise an antisense RNAi oligonucleotide and optionally a sense RNAi oligonucleotide. RNAi compounds may also comprise terminal groups and/or conjugate groups which may be attached to the antisense RNAi oligonucleotide or the sense RNAi oligonucleotide (when present).

Duplexes

RNAi compounds comprising an antisense RNAi oligonucleotide and a sense RNAi oligonucleotide form a duplex, because the sense RNAi oligonucleotide comprises an antisense-hybridizing region that is complementary to the antisense RNAi oligonucleotide. In certain embodiments, each nucleobase of the antisense RNAi oligonucleotide and the sense RNAi oligonucleotide are complementary to one another. In certain embodiments, the two RNAi oligonucleotides have at least one mismatch relative to one another.

In certain embodiments, the antisense hybridizing region constitutes the entire length of the sense RNAi oligonucleotide and the antisense RNAi oligonucleotide. In certain embodiments, one or both of the antisense RNAi oligonucleotide and the sense RNAi oligonucleotide comprise additional nucleosides at one or both ends that do not hybridize (overhanging nucleosides). In certain embodiments, overhanging nucleosides are DNA. In certain embodiments, overhanging nucleosides are linked to each other (where there is more than one) and to the first non-overhanging nucleoside with phosphorothioate linkages.

B. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer. In certain embodiments, the modified oligonucleotide is an antisense RNAi oligonucleotide. In certain embodiments, the modified oligonucleotide is a sense RNAi oligonucleotide.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

3. Cell-Targeting Moieties

In certain embodiments, a conjugate group comprises a cell-targeting moiety. In certain embodiments, a conjugate group has the general formula:

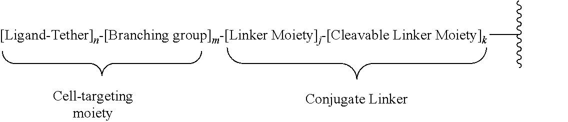

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

C. Certain Terminal Groups

In certain embodiments, oligomeric compounds comprise one or more terminal groups. In certain such embodiments, modified oligonucleotides comprise a phosphorus-containing group at the 5′-end of the modified oligonucleotide. In certain embodiments, the phosphorus-containing group is at the 5′-end of the antisense RNAi oligonucleotide and/or the sense RNAi oligonucleotide. In certain embodiments, the terminal group is a phosphate stabilized phosphate group. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos) or 5′-deoxy-5′-C-malonyl. When the 5′-end phosphorus-containing group is 5′-end vinylphosphonate, the 5′VP can be either 5′-E-VP isomer (i.e., trans-vinylphosphonate), 5′-Z-VP isomer (i.e., cis-vinylphosphonate), or mixtures thereof. Although such phosphate group can be attached to any modified oligonucleotide, it has particularly been shown that attachment of such a group to an antisense RNAi oligonucleotide improves activity of certain RNAi compounds. See, e.g., Prakash et al., Nucleic Acids Res., 43(6):2993-3011, 2015; Elkayam, et al., Nucleic Acids Res., 45(6):3528-3536, 2017; Parmar, et al. ChemBioChem, 17(11)985-989; 2016; Harastzi, et al., Nucleic Acids Res., 45(13):7581-7592, 2017. In certain embodiments, the phosphate stabilizing group is 5′-cyclopropyl phosphonate. See e.g., WO/2018/027106.

In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

D. Certain Specific RNAi Motifs

RNAi compounds can be described by motif or by specific features.

In certain embodiments, the RNAi compounds described herein comprise:
(a) a sense RNAi oligonucleotide having:
   (i) a length of 21 nucleotides;
   (ii) a conjugate attached to the 3′-end; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end); and
(b) an antisense RNAi oligonucleotide having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleoside linkages between nucleoside positions 21 and 22, and between nucleoside positions 22 and 23 (counting from the 5′ end);
   wherein the two nucleotides at the 3′end of the antisense RNAi oligonucleotide are overhanging nucleosides, and the end of the RNAi compound duplex constituting the 5′-end of the antisense RNAi oligonucleotide and the 3′-end of the sense RNAi oligonucleotide is blunt (i.e., neither oligonucleotide has overhang nucleoside at that end and instead the hybridizing region of the sense RNAi oligonucleotide includes the 3′-most nucleoside of the sense RNAi oligonucleotide and that nucleoside hybridizes with the 5′-most nucleoside of the antisense oligonucleotide).

In certain embodiments, the RNAi compounds described herein comprise:
(a) a sense RNAi oligonucleotide having:
   (i) a length of 21 nucleotides;
   (ii) a conjugate attached to the 3′-end;
   (iii) 2′-F modifications at positions 1, 3, 5, 7, 9 to 11, 13, 17, 19, and 21, and 2′-OMe modifications at positions 2, 4, 6, 8, 12, 14, 16, 18, and 20 (counting from the 5′ end); and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi compounds described herein comprise:
(a) a sense RNAi oligonucleotide having:
   (i) a length of 21 nucleotides;
   (ii) a conjugate attached to the 3′-end;
   (iii) 2′-OMe modifications at positions 1 to 6, 8, 10, and 12 to 21, and 2′-F modifications at positions 7 and 9, and a deoxynucleotide at position 11 (counting from the 5′ end); and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3, 7, 9, 11, 13, 15, 17, and 19 to 23, and 2′F modifications at positions 2, 4 to 6, 8, 10, 12, 14, 16, and 18 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex has a two nucleotide overhang at the 3′end of the antisense RNAi oligonucleotide, and a blunt end at the 5′-end of the antisense RNAi oligonucleotide.

In certain embodiments, the RNAi compounds described herein comprise:
(a) a sense RNAi oligonucleotide having:
   (i) a length of 21 nucleotides;
   (ii) a conjugate attached to the 3′-end;
   (iii) 2′-OMe modifications at positions 1 to 6, 8, and 12 to 21, and 2′-F modifications at positions 7, and 9 to 11; and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3 to 5, 7, 8, 10 to 13, 15, and 17 to 23, and 2′F modifications at positions 2, 6, 9, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex has a two nucleotide overhang at the 3′end of the antisense RNAi oligonucleotide, and a blunt end at the 5′-end of the antisense RNAi oligonucleotide.

In certain embodiments, the RNAi compounds described herein comprise:
(a) a sense RNAi oligonucleotide having:
   (i) a length of 21 nucleotides;
   (ii) a conjugate attached to the 3′-end;
   (iii) 2′-OMe modifications at positions 1 to 6, 8, and 12 to 21, and 2′-F modifications at positions 7, and 9 to 11; and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3 to 5, 7, 10 to 13, 15, and 17 to 23, and 2′F modifications at positions 2, 6, 8, 9, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
wherein the RNAi duplex has a two nucleotide overhang at the 3′end of the antisense RNAi oligonucleotide, and a blunt end at the 5′-end of the antisense RNAi oligonucleotide.
   In certain embodiments, the RNAi compounds described herein comprise:
(a) a sense RNAi oligonucleotide having:
   (i) a length of 19 nucleotides;
   (ii) a conjugate attached to the 3′-end;
   (iii) 2′-OMe modifications at positions 1 to 4, 6, and 10 to 19, and 2′-F modifications at positions 5, and 7 to 9; and
   (iv) phosphorothioate internucleoside linkages between nucleoside positions 1 and 2, and between nucleoside positions 2 and 3 (counting from the 5′ end);
and
(b) an antisense RNAi oligonucleotide having:
   (i) a length of 21 nucleotides;
   (ii) 2′-OMe modifications at positions 1, 3 to 5, 7, 10 to 13, 15, and 17 to 21, and 2′F modifications at positions 2, 6, 8, 9, 14, and 16 (counting from the 5′ end); and
   (iii) phosphorothioate internucleoside linkages between nucleoside positions 1 and 2, between nucleoside positions 2 and 3, between nucleoside positions 19 and 20, and between nucleoside positions 20 and 21 (counting from the 5′ end);
   wherein the RNAi duplex has a two nucleotide overhang at the 3′end of the antisense RNAi oligonucleotide, and a blunt end at the 5′-end of the antisense RNAi oligonucleotide.

In certain embodiments, the RNAi compounds described herein comprise:
(a) a sense RNAi oligonucleotide having:
   (i) a length of 21 nucleotides;
   (ii) a conjugate attached at position 6 (counting from the 5′ end);
   (iii) 2′-F modifications at positions 7 and 9 to 11, and 2′-OMe modifications at positions 1 to 5, 8, and 12 to 21 (counting from the 5′ end); and
   (iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);
and
(b) an antisense RNAi oligonucleotide having:
   (i) a length of 23 nucleotides;
   (ii) 2′-OMe modifications at positions 1, 3 to 5, 7, 10 to 13, 15, and 17 to 23, and 2′F modifications at positions 2, 6, 8, 9, 14, and 16 (counting from the 5′ end);
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end); and
   (iv) a stabilized phosphate group attached to the 5′ position of the 5′-most nucleoside;
   wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi compounds described herein comprise:
(a) a sense RNAi oligonucleotide having:
   (i) a length of 21 nucleotides;
   (ii) a conjugate attached to the 3′-end;
   (iii) 2′-F modifications at positions 7 and 9 to 11, and 2′-OMe modifications at positions 1 to 6, 8, and 12 to 21 (counting from the 5′ end);
   (iv) phosphorothioate internucleoside linkages between nucleoside positions 1 and 2 and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3 to 5, 7 to 13, 15, and 17 to 23 an (S)-GNA modification at position 6, and 2′F modifications at positions 2, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi compounds described herein comprise:
(a) a sense RNAi oligonucleotide having:
   (i) a length of 21 nucleotides;
   (ii) a conjugate attached to the 3′-end;
   (iii) 2′-F modifications at positions 7 and 9 to 11, and 2′-OMe modifications at positions 1 to 6, 8, and 12 to 21 (counting from the 5′ end);
   (iv) phosphorothioate internucleoside linkages between nucleoside positions 1 and 2 and between nucleoside positions 2 and 3 (counting from the 5′ end);
and
(b) an antisense RNAi oligonucleotide having:
   (i) a length of 23 nucleotides;
   (ii) 2′-OMe modifications at positions 1, 3 to 6, 8 to 13, 15, and 17 to 23 an (S)-GNA modification at position 7, and 2′F modifications at positions 2, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi compounds described herein comprise:
(a) a sense RNAi oligonucleotide having:
   (i) a length of 21 nucleotides;
   (ii) a conjugate attached at position 6 (counting from the 5′ end); and
   (iii) 2′-F modifications at positions 7 and 9 to 11, and 2′-OMe modifications at positions 1 to 5, 8, and 12 to 21 (counting from the 5′ end);
   (iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);
and
(b) an antisense RNAi oligonucleotide having:
   (i) a length of 23 nucleotides;
   (ii) 2′-OMe modifications at positions 1, 3 to 5, 7 to 13, 15, and 17 to 23 an (S)-GNA modification at position 6, and 2′F modifications at positions 2, 14, and 16 (counting from the 5′ end);

(iii) phosphorothioate internucleoside linkages between nucleoside positions 1 and 2, between nucleoside positions 2 and 3, between nucleoside positions 21 and 22, and between nucleoside positions 22 and 23 (counting from the 5′ end); and

(iv) a stabilized phosphate group attached to the 5′ position of the 5′-most nucleoside; wherein the RNAi duplex includes a two nucleotide overhang at the 3′end of the antisense RNAi oligonucleotide, and a blunt end at the 5′-end of the antisense RNAi oligonucleotide.

In certain embodiments, the RNAi compounds described herein comprise:
(a) a sense RNAi oligonucleotide having:
   (i) a length of 21 nucleotides;
   (ii) a conjugate attached at position 6 (counting from the 5′ end);
   (iii) 2′-F modifications at positions 7 and 9 to 11, and 2′-OMe modifications at positions 1 to 5, 8, and 12 to 21 (counting from the 5′ end); and
   (iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);
and
(b) an antisense RNAi oligonucleotide having:
   (i) a length of 23 nucleotides;
   (ii) 2′-OMe modifications at positions 1, 3 to 6, 8 to 13, 15, and 17 to 23 an (S)-GNA modification at position 7, and 2′F modifications at positions 2, 14, and 16 (counting from the 5′ end);
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end); and
   (iv) a stabilized phosphate group attached to the 5′ position of the 5′-most nucleoside;
   wherein the two nucleotides at the 3′end of the antisense RNAi oligonucleotide are overhanging nucleosides, and the end of the RNAi compound duplex constituting the 5′-end of the antisense RNAi oligonucleotide and the 3′-end of the sense RNAi oligonucleotide is blunt (i.e., neither oligonucleotide has overhang nucleoside at that end and instead the hybridizing region of the sense RNAi oligonucleotide includes the 3′-most nucleoside of the sense RNAi oligonucleotide and that nucleoside hybridizes with the 5′-most nucleoside of the antisense oligonucleotide).

In certain embodiments, the RNAi compounds described herein comprise:
(a) a sense RNAi oligonucleotide having:
   (i) a length of 21 nucleotides;
   (ii) a conjugate attached to the 5′-end;
   (iii) 2′-OMe modifications at positions 1 to 8, and 12 to 21, and 2′-F modifications at positions 9 to 11; and
   (iv) inverted abasic sugar moieties attached to both the 5′-most and 3′-most nucleosides;
and
(b) an antisense RNAi oligonucleotide having:
   (i) a length of 21 nucleotides;
   (ii) 2′-OMe modifications at positions 1, 3, 5, 7, 9, 11, 13, 15, 17, 19, and 21, and 2′F modifications at positions 2, 4, 6, 8, 10, 12, 14, 16, 18, and 20 (counting from the 5′ end); and
   (iii) phosphorothioate internucleoside linkages between nucleoside positions 1 and 2, between nucleoside positions 2 and 3, between nucleoside positions 3 and 4, and between nucleoside positions 20 and 21 (counting from the 5′ end).

In certain embodiments, the RNAi compounds described herein comprise:
(a) a sense RNAi oligonucleotide having:
   (i) a length of 21 nucleotides;
   (ii) a conjugate attached to the 5′-end;
   (iii) 2′-OMe modifications at positions 1 to 8, and 12 to 21, and 2′-F modifications at positions 9 to 11;
   (iv) a phosphorothioate internucleoside linkage between nucleoside positions 1 and 2 (counting from the 5′ end); and
   (v) an inverted abasic sugar moiety attached to the 3′-most nucleoside;
and
(b) an antisense RNAi oligonucleotide having:
   (i) a length of 21 nucleotides;
   (ii) 2′-OMe modifications at positions 1, 3, 5, 7, 9, 11, 13, 15, 17, 19, and 21, and 2′F modifications at positions 2, 4, 6, 8, 10, 12, 14, 16, 18, and 20 (counting from the 5′ end); and
   (iii) phosphorothioate internucleoside linkages between nucleoside positions 1 and 2, between nucleoside positions 2 and 3, between nucleoside positions 3 and 4, and between nucleoside positions 20 and 21 (counting from the 5′ end).

In certain embodiments, the RNAi compounds described herein comprise:
(a) a sense RNAi oligonucleotide having:
   (i) a length of 19 nucleotides;
   (ii) a conjugate attached to the 5′-end;
   (iii) 2′-OMe modifications at positions 2, 4, 6, 8, 10, 12, 14, 16, 18, and 20, and 2′-F modifications at positions 1, 3, 5, 7, 9, 11, 13, 15, 17, 19, and 21; and
   (iv) phosphorothioate internucleoside linkages between nucleoside positions 17 and 18, and between nucleoside positions 18 and 19 (counting from the 5′ end);
and
(b) an antisense RNAi oligonucleotide having:
   (i) a length of 19 nucleotides;
   (ii) 2′-OMe modifications at positions 1, 3, 5, 7, 9, 11, 13, 15, 17, 19, and 21, and 2′F modifications at positions 2, 4, 6, 8, 10, 12, 14, 16, 18, and 20 (counting from the 5′ end); and
   (iii) phosphorothioate internucleoside linkages between nucleoside positions 1 and 2, between nucleoside positions 2 and 3, between nucleoside positions 17 and 18, and between nucleoside positions 18 and 19 (counting from the 5′ end).

In any of the above embodiments, the conjugate at the 3′-end of the sense RNAi oligonucleotide may comprise a targeting moiety. In certain such embodiments, the targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

In certain embodiments, the RNAi compound comprises a 21 nucleotide sense RNAi oligonucleotide and a 23 nucleotide antisense RNAi oligonucleotide, wherein the sense RNAi oligonucleotide contains at least one motif of three contiguous 2′-F modified nucleosides at positions 9, 10, 11 from the 5′-end; the antisense RNAi oligonucleotide contains at least one motif of three 2′-O-methyl modifications on three consecutive nucleotides at positions 11, 12, 13 from the 5′ end, wherein one end of the RNAi compound is blunt, while the other end comprises a 2 nucleotide overhang. Preferably, the 2 nucleotide overhang is at the 3′-end of the antisense RNAi oligonucleotide.

In certain embodiments, when the 2 nucleotide overhang is at the 3′-end of the antisense RNAi oligonucleotide, there may be two phosphorothioate internucleoside linkages between the terminal three nucleotides, wherein two of the three nucleotides are the overhang nucleotides, and the third nucleotide is a paired nucleotide next to the overhang nucleotide. In certain embodiments, the RNAi compound additionally has two phosphorothioate internucleoside linkages between the terminal three nucleotides at both the 5′-end of the sense RNAi oligonucleotide and at the 5′-end of the antisense RNAi oligonucleotide. In certain embodiments, every nucleotide in the sense RNAi oligonucleotide and the antisense RNAi oligonucleotide of the RNAi compound is a modified nucleotide. In certain embodiments, each nucleotide is independently modified with a 2′-O-methyl or 3′-fluoro, e.g. in an alternating motif. Optionally, the RNAi compound comprises a conjugate.

In certain embodiments, every nucleotide in the sense RNAi oligonucleotide and antisense RNAi oligonucleotide of the RNAi compound, including the nucleotides that are part of the motifs, may be modified. Each nucleotide may be modified with the same or different modification, which can include one or more alteration of one or both of the non-linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

In certain embodiments, each nucleoside of the sense RNAi oligonucleotide and antisense RNAi oligonucleotide is independently modified with LNA, cEt, UNA, HNA, CeNA, 2′-MOE, 2′-OMe, 2′-O-allyl, 2′-C-allyl, 2′-deoxy, 2′-hydroxyl, or 2′-fluoro. The RNAi compound can contain more than one modification. In one embodiment, each nucleoside of the sense RNAi oligonucleotide and antisense RNAi oligonucleotide is independently modified with 2′-O-methyl or 2′-F. In certain embodiments, the modification is a 2′-NMA modification.

The term “alternating motif” as used herein refers to a motif having one or more modifications, each modification occurring on alternating nucleotides of one RNAi oligonucleotide. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense RNAi oligonucleotide or antisense RNAi oligonucleotide can be selected from several possibilities of modifications within the alternating motif such as “ABABAB . . . ”, “ACACAC . . . ” “BDBDBD . . . ” or “CDCDCD . . . ,” etc.

In certain embodiments, the modification pattern for the alternating motif on the sense RNAi oligonucleotide relative to the modification pattern for the alternating motif on the antisense RNAi oligonucleotide is shifted. The shift may be such that the group of modified nucleotides of the sense RNAi oligonucleotide corresponds to a group of differently modified nucleotides of the antisense RNAi oligonucleotide and vice versa. For example, the sense RNAi oligonucleotide when paired with the antisense RNAi oligonucleotide in the RNAi duplex, the alternating motif in the sense RNAi oligonucleotide may start with “ABABAB” from 5′-3′ of the RNAi oligonucleotide and the alternating motif in the antisense RNAi oligonucleotide may start with “BABABA” from 5′-3 ‘of the RNAi oligonucleotide within the duplex region. As another example, the alternating motif in the sense RNAi oligonucleotide may start with “AABBAABB” from 5’-3′ of the RNAi oligonucleotide and the alternating motif in the antisense RNAi oligonucleotide may start with “BBAABBAA” from 5′-3′ of the RNAi oligonucleotide within the duplex region, so that there is a complete or partial shift of the modification 10 patterns between the sense RNAi oligonucleotide and the antisense RNAi oligonucleotide.

In certain embodiments, the RNAi compound comprising the pattern of the alternating motif of 2′-O-methyl modification and 2′-F modification on the sense RNAi oligonucleotide initially has a shift relative to the pattern of the alternating motif of 2′-O-methyl modification and 2′-F modification on the antisense RNAi oligonucleotide initially, i.e., the 2′-O-methyl modified nucleotide on the sense RNAi oligonucleotide base pairs with a 2′-F modified nucleotides on the antisense RNAi oligonucleotide and vice versa. The 1 position of the sense RNAi oligonucleotide may start with the 2′-F modification, and the 1 position of the antisense RNAi oligonucleotide may start with a 2′-O-methyl modification.

The introduction of one or more motifs of three identical modifications on three consecutive nucleotides to the sense RNAi oligonucleotide and/or antisense RNAi oligonucleotide interrupts the initial modification pattern present in the sense RNAi oligonucleotide and/or antisense RNAi oligonucleotide. This interruption of the modification pattern of the sense and/or antisense RNAi oligonucleotide by introducing one or more motifs of three identical modifications on three consecutive nucleotides to the sense and/or antisense RNAi oligonucleotide surprisingly enhances the gene silencing activity to the target gene. In one embodiment, when the motif of three identical modifications on three consecutive 25 nucleotides is introduced to any of the RNAi oligonucleotide s, the modification of the nucleotide next to the motif is a different modification than the modification of the motif. For example, the portion of the sequence containing the motif is “ . . . NaYYYNb . . . ,” where “Y” represents the modification of the motif of three identical modifications on three consecutive nucleotide, and “Na” and “Nb” represent a modification to the nucleotide next to the motif “YYY” that is different than the modification of Y, and where Na and Nb can be the same or different modifications. Alternatively, Na and/or Nb may be present or absent when there is a wing modification present.

In certain embodiments, the sense RNAi oligonucleotide may be represented by formula (I):

5′nₚ-Nₐ—(X X X)i-N_{b}—Y Y Y—N_{b}—(Z Z Z)ᵣNₐ-n_{q}3′ (I)

wherein:

i and j are each independently 0 or 1;

p and q are each independently 0-6;

each Nₐ independently represents 0-25 linked nucleosides comprising at least two differently modified nucleosides;

each N_{b} independently represents 0-10 linked nucleosides;

each nₚ and n_{q} independently represent an overhanging nucleoside;

wherein N_{b} and Y do not have the same modification; and

XXX, YYY and ZZZ each independently represent modified nucleosides where each X nucleoside has the same modification; each Y nucleoside has the same modification; and each Z nucleoside has the same modification. In certain embodiments, each Y comprises a 2′-F modification.

In certain embodiments, the Nₐ and N_{b} comprise modifications of alternating patterns.

In certain embodiments, the YYY motif occurs at or near the cleavage site of the target nucleic acid. For example, when the RNAi compound has a duplex region of 17-23 nucleotides in length, the YYY motif can occur at or near the vicinity of the cleavage site (e.g., can occur at positions 6, 7, 8; 7, 8, 9; 8, 9, 10; 9, 10, 11; 10, 11, 12; or 11, 12, 13) of the sense RNAi oligonucleotide, the count starting from the 1^{st} nucleotide from the 5′-end; or optionally, the count starting at the 1^{st} paired nucleotide within the duplex region, from the 5′-end.

In certain embodiments, the antisense RNAi oligonucleotide of the RNAi may be represented by the formula:

5′n_{q}-Nₐ′—(Z′Z′Z′)ₖ—N_{b}′—Y′Y′Y′—N_{b}′—(X′X′X′)ₗ—N′ₐ-nₚ3′ (II)

wherein:

k and l are each independently 0 or 1;

p′ and q′ are each independently 0-6;

each Nₐ′ independently represents 0-25 linked nucleotides comprising at least two differently modified nucleotides;

each N_{b}′ independently represents 0-10 linked nucleotides;

each nₚ′ and n_{q}′ independently represent an overhanging nucleoside;

wherein N_{b}′ and Y′ do not have the same modification; and

X′X′X′, Y′Y′Y′ and Z′Z′Z′ each independently represent modified nucleosides where each X′ nucleoside has the same modification; each Y′ nucleoside has the same modification; and each Z′ nucleoside has the same modification. In certain embodiments, each Y′ comprises a 2′-F modification. In certain embodiments, each Y′ comprises a 2′-OMe modification.

In certain embodiments, the Nₐ′ and/or N_{b}′ comprise modifications of alternating patterns.

In certain embodiments, the Y′Y′Y′ motif occurs at or near the cleavage site of the target nucleic acid. For example, when the RNAi compound has a duplex region of 17-23 nucleotides in length, the Y′Y′Y′ motif can occur at positions 9, 10, 11; 10, 11, 12; 11, 12, 13; 12, 13, 14; or 13, 14, 15 of the antisense RNAi oligonucleotide, with the count starting from the 1^{st} nucleotide from the 5′-end; or, optionally, the count starting at the 1^{st} paired nucleotide within the duplex region, from the 5′-end. Preferably, the Y′Y′Y′ motif occurs at positions 11, 12, 13.

In certain embodiments, k is 1 and 1 is 0, or k is 0 and 1 is 1, or both k and l are 1.

The antisense RNAi oligonucleotide can therefore be represented by the following formulas:

5′n_{g}′-Nₐ′—Z′Z′Z′—N_{b}′—Y′Y′Y′—Nₐ′-nₚ′3′ (IIb);

5′ n_{g}′-Nₐ′—Y′Y′Y′—N_{b}′—X′ X′X′-nₚ′3′ (IIc); or

5′ n_{g}′-Na— Z′Z′Z′—N_{b}′—Y′Y′Y′—N_{b}′— X′X′X′—Nₐ′-nₚ′3′ (IId).

When the antisense RNAi oligonucleotide is represented by formula IIb, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense RNAi oligonucleotide is represented by formula IIc, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense RNAi oligonucleotide is represented by formula IId, N_{b}′ represents 0-10, 0-7, 0-5, 0-4, 0-2, or 0 linked nucleosides. Each Nₐ′ independently represents 2-20, 2-15, or 2-10 linked nucleosides.

Preferably, N_{b}′ is 0, 1, 2, 3, 4, 5, or 6.

In certain embodiments, k is 0 and 1 is 0 and the antisense RNAi oligonucleotide may be represented by the formula:

5′ nₚ′-Na′—Y′Y′Y′—Nₐ′-n_{q}′3′ (Ia).

When the antisense RNAi oligonucleotide is represented by formula IIa, each Nₐ′ independently represents 2-20, 2-15, or 2-10 linked nucleosides.

Each X′, Y′, and Z′ may be the same or different from each other.

Each nucleotide of the sense RNAi oligonucleotide and antisense RNAi oligonucleotide may be independently modified with LNA, UNA, cEt, HNA, CeNA, 2′-methoxyethyl, 2′-O-methyl, 2′-O-allyl, 2′-C-allyl, 2′-hydroxyl, or 2′-fluoro. For example, each nucleotide of the sense RNAi oligonucleotide and antisense RNAi oligonucleotide is independently modified with, 2′-O-methyl or 2′-fluoro. Each X, Y, Z, X′, Y′, and Z′, in particular, may represent a 2′-O-methyl modification or 2′-fluoro modification. In certain embodiments, the modification is a 2′-NMA modification.

In certain embodiments, the sense RNAi oligonucleotide of the RNAi compound may contain YYY motif occurring at 9, 10, and 11 positions of the RNAi oligonucleotide when the duplex region is 21 nucleotides, the count starting from the 1^{st} nucleotide from the 5′-end, or optionally, the count starting at the 1^{st} paired nucleotide within the duplex region, from the 5′-end; and Y represents 2′-F modification. The sense RNAi oligonucleotide may additionally contain XXX motif or ZZZ motifs as wing modifications at the opposite end of the duplex region; and XXX and ZZZ each independently represents a 2′-O-methyl modification or 2′-fluoro modification.

In certain embodiments, the antisense RNAi oligonucleotide may contain Y′Y′Y′ motif occurring at positions 11, 12, 13 of the RNAi oligonucleotide, the count starting from the 1^{st} nucleotide from the 5′-end, or optionally, the count starting at the 1^{st} paired nucleotide within the duplex region, from the 5′-end; and Y′ represents 2′-O-methyl modification. The antisense RNAi oligonucleotide may additionally contain X′X′X′ motif or Z′Z′Z′ motif as wing modifications at the opposite end of the duplex region; and X′X′X′ or Z′Z′Z′ each independently represents a 2′-O-methyl modification or 2′-fluoro modification.

The sense RNAi oligonucleotide represented by any one of the above formulas Ia, Ib, Ic, and Id forms a duplex with an antisense RNAi oligonucleotide being represented by any one of the formulas IIa, IIb, IIc, and IId, respectively.

Accordingly, the RNAi compounds described herein may comprise a sense RNAi oligonucleotide and an antisense RNAi oligonucleotide, each RNAi oligonucleotide having 14 to 30 nucleotides, the RNAi duplex represented by formula (III):

Sense: 5′nₚ-Nₐ—(XXX)ᵢ—N_{b}—YYY—N_{b}—(ZZZ)ⱼ—Nₐ-n_{q}3′

Antisense: 3′ nₚ′-Nₐ′—(X′X′X′)ₖ—N_{b}′—Y′Y′Y′—N_{b}′—(Z′Z′Z′)ₗ—Nₐ′-n_{q}′5′

wherein:

i, j, k, and 1 are each independently 0 or 1;

p, p′, q, and q′ are each independently 0-6;

each Nₐ and Nₐ′ independently represents 0-25 linked nucleosides, each sequence comprising at least two differently modified nucleotides;

each N_{b} and N_{b}′ independently represents 0-10 linked nucleosides;

wherein each nₚ′, nₚ, n_{q}′ and n_{q}, each of which may or may not be present, independently represents an overhang nucleotide; and

XXX, YYY, X′X′X′, Y′Y′Y′, and Z′Z′Z′ each independently represent one motif of three identical modifications on three consecutive nucleotides.

In certain embodiments, i is 0 and j is 0; or i is 1 and j is 0; or i is 0 and j is 1; or both i and j are 0; or both i and j are 1. In another embodiment, k is 0 and 1 is 0; or k is 1 and 1 is 0, or k is 0 and 1 is 1; or both k and 1 are 0; or both k and l are 1.

Exemplary combinations of the sense RNAi oligonucleotide and antisense RNAi oligonucleotide forming a RNAi duplex include the formulas below:

5′nₚ-Nₐ—Y Y Y—Nₐ-n_{q}3′

3′ nₚ′-Nₐ′—Y′Y′Y′—Nₐ′n_{q}′5′ (IIIa)

5′nₚ—Nₐ—Y Y Y—N_{b}—Z Z Z—Nₐ-n_{q}3′

3′ nₚ′-Nₐ′—Y′Y′Y′—Nb′—Z′Z′Z′—Nₐ′n_{q}′5′ (IIIb)

5′np-Nₐ—X X X—N_{b}—Y Y Y—Nₐ-n_{q}3′

3′np′-Nₐ′—X′X′X′—N_{b}′—Y′Y′Y′—Nₐ′-n_{q}5′ (IIIc)

5′np-Nₐ—X X X—N_{b}—Y Y Y—N_{b}—Z Z Z—Nₐ-n_{q}3′

3′ np′-Nₐ′—X′X′X′—N_{b}′—Y′Y′Y′—N_{b}′—Z′Z′Z′—Nₐ-n_{q}′5′ (IIId)

When the RNAi compound is represented with formula Ma, each Nₐ independently represents 2-20, 2-15, or 2-10 linked nucleosides.

When the RNAi compound is represented with formula IIIb, each N_{b} independently represents 1-10, 1-7, 1-5, or 1-4 linked nucleosides. Each Nₐ independently represents 2-20, 2-15, or 2-10 linked nucleosides.

When the RNAi compound is represented with formula IIIc, each N_{b}, N_{b}′ independently represents 0-10, 0-7, 0-10, 0-7, 0-5, 0-4, 0-2, or 0 linked nucleosides. Each Nₐ independently represents 2-20, 2-15, or 2-10 linked nucleosides.

When the RNAi compound is represented with formula IIId, each N_{b}, N_{b}′ independently represents 0-10, 0-7, 0-10, 0-7, 0-5, 0-4, 0-2, or 0 linked nucleosides. Each Nₐ, Nₐ′ independently 2-20, 2-15, or 2-10 linked nucleosides. Each Nₐ, Nₐ′, N_{b}, N_{b}′ independently comprises modifications of alternating pattern.

Each of X, Y, and Z in formulas III, IIIa, IIIb, IIIc, and IIId may be the same or different from each other.

When the RNAi compound is represented by formula III, IIIa, IIIb, IIIc, and/or IIId, at least one of the Y nucleotides may form a base pair with one of the Y′ nucleotides. Alternatively, at least two of the Y nucleotides may form base pairs with the corresponding Y′ nucleotides; or all three of the Y nucleotides may form base pairs with the corresponding Y′ nucleotides.

When the RNAi compound is represented by formula IIIb or IIId, at least one of the Z nucleotides may form a base pair with one of the Z′ nucleotides. Alternatively, at least two of the Z nucleotides may form base pairs with the corresponding Z′ nucleotides; or all three of the Z nucleotides may form base pairs with the corresponding Z′ nucleotides.

When the RNAi compound is represented by formula IIIc or IIId, at least one of the X nucleotides may form a base pair with one of the X′ nucleotides. Alternatively, at least two of the X nucleotides may form base pairs with the corresponding X′ nucleotides; or all three of the X nucleotides may form base pairs with the corresponding X′ nucleotides.

In certain embodiments, the modification of the Y nucleotide is different than the modification on the Y′ nucleotide, the modification on the Z nucleotide is different than the modification on the Z′ nucleotide, and/or the modification on the X nucleotide is different than the modification on the X′ nucleotide.

In certain embodiments, when the RNAi compound is represented by the formula IIId, the Nₐ modifications are 2′-O-methyl or 2′-fluoro modifications. In another embodiment, when the RNAi compound is represented by formula IIId, the Nₐ modifications are 2′-O-methyl or 2′-fluoro modifications and nₚ′>0 and at least one nₚ′ is linked to a neighboring nucleotide via phosphorothioate linkage. In other embodiments, when the RNAi compound is represented by formula IIId, the Nₐ modifications are 2′-O-methyl or 2′-fluoro modifications, nₚ′>0 and at least one nₚ′ is linked to a neighboring nucleotide via phosphorothioate linkage, and the sense RNAi oligonucleotide is conjugated to one or more cell targeting group attached through a bivalent or trivalent branched linker. In certain embodiments, when the RNAi compound is represented by formula IIId, the Nₐ modifications are 2′-O-methyl or 2′-fluoro modifications, nₚ′>0 and at least one nₚ′ is linked to a neighboring nucleotide via phosphorothioate linkage, the sense RNAi oligonucleotide comprises at least one phosphorothioate linkage and the sense RNAi oligonucleotide is conjugated to one or more cell targeting group attached through a bivalent or trivalent branched linker.

In certain embodiments, when the RNAi compound is represented by the formula Ma, the Nₐ modifications are 2′-O-methyl or 2′-fluoro modifications and nₚ′>0 and at least one nₚ′ is linked to a neighboring nucleotide via phosphorothioate linkage, the sense RNAi oligonucleotide comprises at least one phosphorothioate linkage and the sense RNAi oligonucleotide is conjugated to one or more cell targeting group attached through a bivalent or trivalent branched linker.

In certain embodiments, the modification is a 2′-NMA modification.

In certain embodiments, the antisense strand may comprise a stabilized phosphate group attached to the 5′ position of the 5′-most nucleoside. In certain embodiments, the stabilized phosphate group comprises an (E)-vinyl phosphonate. In certain embodiments, the stabilized phosphate group comprises a cyclopropyl phosphonate.

In certain embodiments, the antisense strand may comprise a seed-pairing destabilizing modification. In certain embodiments, the seed-pairing destabilizing modification is located at position 6 (counting from the 5′ end). In certain embodiments, the seed-pairing destabilizing modification is located at position 7 (counting from the 5′ end). In certain embodiments, the seed-pairing destabilizing modification is a GNA sugar surrogate. In certain embodiments, the seed-pairing destabilizing modification is an (S)-GNA. In certain embodiments, the seed-pairing destabilizing modification is a UNA. In certain embodiments, the seed-pairing destabilizing modification is a morpholino.

In certain embodiments, the sense strand may comprise an inverted abasic sugar moiety attached to the 5′-most nucleoside. In certain embodiments, the sense strand may comprise an inverted abasic sugar moiety attached to the 3′-most nucleoside. In certain embodiments, the sense strand may comprise inverted abasic sugar moieties attached to both the 5′-most and 3′-most nucleosides.

In certain embodiments, the sense strand may comprise a conjugate attached at position 6 (counting from the 5′ end). In certain embodiments, the conjugate is attached at the 2′ position of the nucleoside. In certain embodiments the conjugate is a C₁₆ lipid conjugate. In certain embodiments, the modified nucleoside at position 6 of the sense strand has a 2′-O-hexadecyl modified sugar moiety.

### IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA or dsRNAi) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain embodiments, the target non-coding RNA is selected from: a long non-coding RNA, a short non-coding RNA, an intronic RNA molecule.

A. Complementarity/Mismatches to the Target Nucleic Acid and Duplex Complementarity

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

### Gapmer Oligonucleotides

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### Antisense RNAi Oligonucleotides

In certain embodiments, antisense RNAi oligonucleotides comprise one or more mismatched nucleobases relative to the target nucleic acid. In certain embodiments, RNAi activity against the target is reduced by such mismatch, but activity against a non-target is reduced by a greater amount. Thus, in certain embodiments selectivity of the antisense RNAi oligonucleotides is improved.

In certain embodiments, antisense RNAi oligonucleotides comprise a targeting region complementary to the target nucleic acid. In certain embodiments, the targeting region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 25 or at least 25 contiguous nucleotides. In certain embodiments, the targeting region constitutes 70%, 80%, 85%, 90%, 95% of the nucleosides of the antisense RNAi oligonucleotide. In certain embodiments, the targeting region constitutes all of the nucleosides of the antisense RNAi oligonucleotide. In certain embodiments, the targeting region of the antisense RNAi oligonucleotide is at least 99%, 95%, 90%, 85%, or 80% complementary to the target nucleic acid. In certain embodiments, the targeting region of the antisense RNAi oligonucleotide is 100% complementary to the target nucleic acid.

### Sense RNAi Oligonucleotides

In certain embodiments, RNAi compounds comprise a sense RNAi oligonucleotide. In such embodiments, sense RNAi oligonucleotides comprise an antisense hybridizing region complementary to the antisense RNAi oligonucleotide. In certain embodiments, the antisense hybridizing region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 25 or at least 25 contiguous nucleotides. In certain embodiments, the antisense hybridizing region constitutes 70%, 80%, 85%, 90%, 95% of the nucleosides of the sense RNAi oligonucleotide. In certain embodiments, the antisense hybridizing region constitutes all of the nucleosides of the sense RNAi oligonucleotide. In certain embodiments, the antisense hybridizing region of the sense RNAi oligonucleotide is at least 99%, 95%, 90%, 85%, or 80% complementary to the antisense RNAi oligonucleotide. In certain embodiments, the antisense hybridizing region of the sense RNAi oligonucleotide is 100% complementary to the antisense RNAi oligonucleotide.

The hybridizing region of a sense RNAi oligonucleotide hybridizes with the antisense RNAi oligonucleotide to form a duplex region. In certain embodiments, such duplex region consists of 7 hybridized pairs of nucleosides (one of each pair being on the antisense RNAi oligonucleotide and the other of each pair bien on the sense RNAi oligonucleotide). In certain embodiments, a duplex region comprises least 8, at least 9, at least 10, at least 11, at least 12, at least 13, at least 14, at least 15, at least 16, at least 17, at least 18, at least 19, at least 20, at least 21, at least 22, at least 23, at least 25 or at least 25 hybridized pairs. In certain embodiments, each nucleoside of antisense RNAi oligonucleotide is paired in the duplex region (i.e., the antisense RNAi oligonucleotide has no overhanging nucleosides). In certain embodiments, the antisense RNAi oligonucleotide includes unpaired nucleosides at the 3′-end and/or the 5′end (overhanging nucleosides). In certain embodiments, each nucleoside of sense RNAi oligonucleotide is paired in the duplex region (i.e., the sense RNAi oligonucleotide has no overhanging nucleosides). In certain embodiments, the sense RNAi oligonucleotide includes unpaired nucleosides at the 3′-end and/or the 5′end (overhanging nucleosides). In certain embodiments, duplexes formed by the antisense RNAi oligonucleotide and the sense RNAi oligonucleotide do not include any overhangs at one or both ends. Such ends without overhangs are referred to as blunt. In certain embodiments wherein the antisense RNAi oligonucleotide has overhanging nucleosides, one or more of those overhanging nucleosides are complementary to the target nucleic acid. In certain embodiments wherein the antisense RNAi oligonucleotide has overhanging nucleosides, one or more of those overhanging nucleosides are not complementary to the target nucleic acid.

B. APP

In certain embodiments, oligomeric compounds comprise or consist of an oligonucleotide comprising a region that is complementary to a target nucleic acid, wherein the target nucleic acid is APP. In certain embodiments, APP nucleic acid has the sequence set forth SEQ ID NO: 1 (the cDNA of Ensembl transcript ENST00000346798.7) or the complement of SEQ ID NO: 2 (GENBANK Accession No. NC_000021.9 truncated from nucleotides 25878001 to 26174000). In certain embodiments, APP nucleic acid has the sequence set forth in any of known splice variants of APP, including but not limited to SEQ ID NO: 3 (the cDNA of Ensembl transcript ENST00000357903.7), SEQ ID NO: 4 (the cDNA of Ensembl transcript ENST00000348990.9), SEQ ID NO: 5 (the cDNA of Ensembl transcript ENST00000440126.7), SEQ ID NO: 6 (the cDNA of Ensembl transcript ENST00000354192.7), and/or SEQ ID NO: 7 (the cDNA of Ensembl transcript ENST00000358918.7). In certain embodiments, contacting a cell with an oligomeric compound complementary to SEQ ID NO: 1, SEQ ID NO: 2, SEQ ID NO: 3, SEQ ID NO: 4, SEQ ID NO: 5, SEQ ID NO: 6, or SEQ ID NO: 7 reduces the amount of APP RNA, and in certain embodiments reduces the amount of APP protein. In certain embodiments, the oligomeric compound consists of a modified oligonucleotide. In certain embodiments, contacting a cell with an oligomeric compound complementary to SEQ ID NO: 1, SEQ ID NO: 2, SEQ ID NO: 3, SEQ ID NO: 4, SEQ ID NO: 5, SEQ ID NO: 6, or SEQ ID NO: 7 results in reduced aggregation of β-amyloid. In certain embodiments, the oligomeric compound consists of a modified oligonucleotide. In certain embodiments, the oligomeric compound consists of a modified oligonucleotide and a conjugate group.

C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system. Such tissues include the cortex, spinal cord, and the hippocampus.

### VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons are nevertheless counted toward the weight of the dose, and the mass of the Na+ ions are not counted toward the weight of the dose. Thus, for example, a dose, or dosage unit, of 10 mg of a number of fully protonated molecules that weighs 10 mg. This would be equivalent to 10.58 mg of solvent-free, sodium acetate-free, anhydrous sodiated Compound No. 699467 or 10.65 mg of solvent-free, sodium acetate-free, anhydrous sodiated Compound No. 1381709. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### VII. Certain Hotspot Regions

### 1. Nucleobases 3192-9277 of SEQ ID NO: 3

In certain embodiments, nucleobases 3192-3277 of SEQ ID NO: 3 comprise a hotspot region. In certain embodiments, oligomeric compounds or oligomeric duplexes comprise modified oligonucleotides that are complementary within nucleobases 3192-3277 of SEQ ID NO: 3. In certain embodiments, modified oligonucleotides are 23 nucleobases in length. In certain embodiments, modified oligonucleotides are antisense RNAi oligonucleotides. In certain embodiments, the antisense RNAi oligonucleotide has a sugar motif (from 5′ to 3′) of: mfmfmfmfmfmfmfmfmfmfmmm; wherein “m” represents a 2′-O methylribosyl sugar, and the “f” represents a 2′-fluororibosyl sugar; and a linkage motif (from 5′ to 3′) of: ssooooooooooooooooooss; wherein ‘o’ represents a phosphodiester internucleoside linkage and ‘s’ represents a phosphorothioate internucleoside linkage.

The nucleobase sequences of SEQ ID Nos: 821-824 are complementary within nucleobases 3192-3277 of SEQ ID NO: 3.

RNAi compounds 1382120, 1382123, 1382124, and 1382128 comprise an antisense RNAi oligonucleotide that is complementary within nucleobases 3192-3277 of SEQ ID NO: 3.

In certain embodiments, modified oligonucleotides complementary within nucleobases 5635-5677 of SEQ ID NO: 3 achieve at least 92% reduction of APP RNA in vitro in the standard cell assay. In certain embodiments, modified oligonucleotides complementary within nucleobases 5635-5677 of SEQ ID NO: 3 achieve an average of 94% reduction of APP RNA in vitro in the standard cell assay.

### 2. Additional Hotspot Regions

In certain embodiments, the ranges described in the Table below comprise hotspot regions. Each hotspot region begins with the nucleobase of SEQ ID NO: 1 identified in the “Start Site SEQ ID NO: 1” column and ends with the nucleobase of SEQ ID NO: 1 identified in the “Stop Site SEQ ID NO: 1” column. In certain embodiments, oligomeric compounds or oligomeric duplexes comprise modified oligonucleotides that are complementary within any of the hotspot regions 1-47, as defined in the table below. In certain embodiments, modified oligonucleotides are 18 nucleobases in length. In certain embodiments, modified oligonucleotides are 20 nucleobases in length. In certain embodiments, modified oligonucleotides are 23 nucleobases in length. In certain embodiments, both RNAseH-based antisense oligonucleotides and RISC-based RNAi oligomeric duplexes are active within a given hotspot region, as indicated in the table below.

In certain embodiments, oligomeric compounds comprise modified oligonucleotides that are gapmers. In certain embodiments, modified oligonucleotides have the sugar motif eeeeeddddddddkkeee, wherein each “e” is nucleoside comprising a 2′-MOE sugar moiety, each “k” is a nucleoside comprising a cEt sugar moiety, and each “d” is a nucleoside comprising a 2′-β-D-deoxyribosyl sugar moiety. In certain embodiments, modified oligonucleotides have the sugar motif eeeeeddddddddkeeee, wherein each “e” is nucleoside comprising a 2′-MOE sugar moiety, each “k” is a nucleoside comprising a cEt sugar moiety, and each “d” is a nucleoside comprising a 2′-β-D-deoxyribosyl sugar moiety. In certain embodiments, modified oligonucleotides are 5-10-5 MOE gapmers.

In certain embodiments, oligomeric duplexes comprise an antisense RNAi oligonucleotide and a sense RNAi oligonucleotide, wherein, the antisense RNAi oligonucleotide is complementary within a given hotspot region. In certain embodiments, the antisense RNAi oligonucleotide is 23 nucleosides in length; has a sugar motif (from 5′ to 3′) of: mfmfmfmfmfmfmfmfmfmfmmm; wherein “m” represents a 2′-O methylribosyl sugar, and the “f” represents a 2′-fluororibosyl sugar; and a linkage motif (from 5′ to 3′) of: ssooooooooooooooooooss; wherein ‘o’ represents a phosphodiester internucleoside linkage and ‘s’ represents a phosphorothioate internucleoside linkage. The sense RNAi oligonucleotides in each case is 21 nucleosides in length; has a sugar motif (from 5′ to 3′) of: fmfmfmfmfmfmfmfmfmfmf; wherein “m” represents a 2′-O methylribosyl sugar, and the “f” represents a 2′-fluororibosyl sugar; and a linkage motif (from 5′ to 3′) of: ssooooooooooooooooss; wherein ‘o’ represents a phosphodiester internucleoside linkage and ‘s’ represents a phosphorothioate internucleoside linkage.

The nucleobase sequence of the gapmer antisense oligonucleotide listed under “Gapmer Antisense Oligonucleotides”/“Compound ID in range” column in the table below is complementary to SEQ ID NO: 1 within the specified hotspot region. The nucleobase sequence of the gapmer antisense oligonucleotides listed in the “Gapmer Antisense Oligonucleotides”/“SEQ ID NO: in range” column in the table below are complementary to the target sequence, SEQ ID NO: 1, within the specified hotspot region.

The nucleobase sequence of the antisense RNAi oligonculeotide corresponding to the RNAi Compound ID listed under “RNAi Compounds”/“RNAi Compound ID in range” column in the table below is complementary to SEQ ID NO: 1 within the specified hotspot region. The nucleobase sequence of the antisense RNAi oligonucleotide list in the “RNAi Compounds”/“SEQ ID NO: in range” column is complementary to the target sequence, NO: 1, within the specified hotspot region.

In certain embodiments, gapmers complementary to nucleobases within the hotspot region achieve at least “Gapmer Antisense Oligonucleotides”/“Min. % Red.” (minimum % reduction, relative to untreated control cells) of APP RNA in vitro in the standard cell assay, as indicated in the table below. In certain embodiments, modified oligonucleotides complementary to nucleobases within the hotspot region achieve an average of “Gapmer Antisense Oligonucleotides”/“Avg. % Red.” (average % reduction, relative to untreated control cells) of APP RNA in vitro in the standard cell assay, as indicated in the table below. In certain embodiments, modified oligonucleotides complementary to nucleobases within the hotspot region achieve a maximum of “Gapmer Antisense Oligonucleotides”/“Max. % Red.” (maximum % reduction, relative to untreated control cells) of APP RNA in vitro in the standard cell assay, as indicated in the table below.

In certain embodiments, RNAi oligomeric duplexes having an antisense RNAi oligonucleotide complementary to nucleobases within the hotspot region achieve at least “RNAi Compounds”/“Min. % Red. RNAi” (minimum % reduction, relative to untreated control cells) of APP RNA in vitro in the standard cell assay, as indicated in the table below. In certain embodiments, RNAi oligomeric duplexes having an antisense RNAi oligonucleotide complementary to nucleobases within the hotspot region achieve an average of “RNAi Compounds”/“Avg. % Red.” (average % reduction, relative to untreated control cells) of APP RNA in vitro in the standard cell assay, as indicated in the table below. In certain embodiments, RNAi oligomeric duplexes having an antisense RNAi oligonucleotide complementary to nucleobases within the hotspot region achieve a maximum of “RNAi Compounds”/“Max. % Red. RNAi” (maximum % reduction, relative to untreated control cells) of APP RNA in vitro in the standard cell assay, as indicated in the table below.

TABLE 1a
APP Hotspot Activity

| | | | Gapmer Antisense | | | | | |
|---|---|---|---|---|---|---|---|---|
| Hotspot | Start Site | Stop Site | Oligonucleotides | | | RNAi Compounds | | |
| Region | SEQ ID | SEQ ID | Min. % | Max. % | Avg. % | Min. % | Max. % | Avg. % |
| ID | NO: 1 | NO: 1 | Red. | Red. | Red. | Red. | Red. | Red. |
| 1 | 40 | 78 | 54 | 74 | 60 | 8 | 8 | 8 |
| 2 | 69 | 146 | 41 | 69 | 53 | 8 | 83 | 53 |
| 3 | 83 | 246 | 40 | 77 | 56 | 62 | 93 | 78 |
| 4 | 94 | 225 | 45 | 77 | 58 | 62 | 93 | 81 |
| 5 | 83 | 129 | 41 | 69 | 53 | 67 | 67 | 67 |
| 6 | 194 | 231 | 45 | 75 | 58 | 80 | 80 | 80 |
| 7 | 194 | 238 | 40 | 75 | 57 | 80 | 80 | 80 |
| 8 | 236 | 268 | 46 | 76 | 62 | 92 | 92 | 92 |
| 9 | 258 | 288 | 48 | 81 | 66 | 82 | 82 | 82 |
| 10 | 285 | 311 | 46 | 59 | 51 | 89 | 89 | 89 |
| 11 | 296 | 321 | 46 | 76 | 61 | n/a | n/a | n/a |
| 12 | 307 | 330 | 41 | 60 | 50 | 76 | 76 | 76 |
| 13 | 330 | 352 | 34 | 64 | 55 | n/a | n/a | n/a |
| 14 | 329 | 352 | 33 | 64 | 51 | 65 | 65 | 65 |
| 15 | 339 | 383 | 38 | 81 | 56 | 50 | 50 | 50 |
| 16 | 413 | 477 | 23 | 74 | 55 | 30 | 90 | 68 |
| 17 | 415 | 477 | 23 | 74 | 55 | n/a | n/a | n/a |
| 18 | 415 | 439 | 57 | 65 | 62 | n/a | n/a | n/a |
| 19 | 477 | 506 | 1 | 71 | 52 | n/a | n/a | n/a |
| 20 | 477 | 523 | 1 | 81 | 59 | 92 | 92 | 92 |
| 21 | 477 | 541 | 1 | 81 | 59 | 92 | 97 | 95 |
| 22 | 530 | 557 | 56 | 70 | 65 | n/a | n/a | n/a |
| 23 | 581 | 638 | 71 | 76 | 73 | 80 | 91 | 85 |
| 24 | 636 | 661 | 55 | 84 | 68 | 24 | 24 | 24 |
| 25 | 652 | 697 | 1 | 85 | 62 | 79 | 79 | 79 |
| 26 | 728 | 821 | 58 | 76 | 67 | 65 | 86 | 78 |
| 27 | 770 | 821 | 58 | 58 | 58 | 65 | 85 | 75 |
| 28 | 920 | 950 | 41 | 67 | 53 | 19 | 19 | 19 |
| 29 | 1006 | 1049 | 13 | 62 | 43 | 72 | 72 | 72 |
| 30 | 1152 | 1179 | 40 | 78 | 57 | n/a | n/a | n/a |
| 31 | 1227 | 1274 | 33 | 74 | 50 | 33 | 33 | 33 |
| 32 | 1227 | 1265 | 33 | 74 | 49 | n/a | n/a | n/a |
| 33 | 1268 | 1332 | 0 | 0 | 0 | 85 | 92 | 89 |
| 34 | 1268 | 1311 | n/a | n/a | n/a | 85 | 92 | 88 |
| 35 | 1289 | 1332 | 0 | 0 | 0 | 91 | 92 | 91 |
| 36 | 1518 | 1543 | 39 | 65 | 50 | 28 | 28 | 28 |
| 37 | 1531 | 1593 | 33 | 80 | 55 | 44 | 71 | 57 |
| 38 | 1544 | 1593 | 33 | 80 | 56 | 71 | 71 | 71 |
| 39 | 1634 | 1657 | 0 | 82 | 43 | n/a | n/a | n/a |
| 40 | 1778 | 1800 | 39 | 58 | 51 | n/a | n/a | n/a |
| 41 | 1882 | 1908 | 43 | 90 | 70 | n/a | n/a | n/a |
| 42 | 2051 | 2074 | 51 | 58 | 53 | n/a | n/a | n/a |
| 43 | 2360 | 3117 | n/a | n/a | n/a | 59 | 96 | 88 |
| 44 | 2402 | 3117 | n/a | n/a | n/a | 59 | 96 | 88 |
| 45 | 2360 | 2655 | n/a | n/a | n/a | 83 | 94 | 90 |
| 46 | 2402 | 2655 | n/a | n/a | n/a | 83 | 94 | 90 |
| 47 | 2675 | 3054 | n/a | n/a | n/a | 84 | 96 | 91 |

TABLE 1b
APP Hotspot Compounds and Sequences

| | | | Gapmer Antisense | | | |
|---|---|---|---|---|---|---|
| | | | Oligonucleotides | | RNAi Compounds | |
| | | | | | | SEQ ID NO: |
| Hotspot | Start Site | Stop Site | | | | in range |
| Region | SEQ ID | SEQ ID | Compound ID | SEQ ID NO: | RNAi Compound ID | (Antisense |
| ID | NO: 1 | NO: 1 | in range | in range | in range | Sequence) |
| 1 | 40 | 78 | 828404-828407 | 22, 241, 315, | 1381712 | 668 |
| | | | | 391 | | |
| 2 | 69 | 146 | 828412-828421 | 23, 24, 95, 96, | 1381733, 1381734, | 674, 675, 678 |
| | | | | 170, 171, 243, | 1381740 | |
| | | | | 317, 392, 393 | | |
| 3 | 83 | 246 | 828413-828429 | 12, 23, 24, 95, | 1381733, 1381735, | 674, 676, 677, |
| | | | | 96, 97, 170, | 1381736, 1381740, | 678, 682, 686, |
| | | | | 171, 172, 243, | 1381755, 1381771, | 688 |
| | | | | 244, 245, 317, | 1381773 | |
| | | | | 318, 319, 393, | | |
| | | | | 394, 395 | | |
| 4 | 94 | 225 | 828417-828424 | 24, 96, 171, | 1381733, 1381735, | 674, 676, 677, |
| | | | | 244, 317, 318, | 1381736, 1381740, | 678, 682, 686 |
| | | | | 393, 394 | 1381755, 1381771 | |
| 5 | 83 | 129 | 828413-828420 | 23, 24, 95, 96, | 1381733 | 674 |
| | | | | 170, 243, 317, | | |
| | | | | 393 | | |
| 6 | 194 | 231 | 699467, | 12, 97, 172, | 1381771 | 686 |
| | | | 828423-828426 | 318, 394 | | |
| 7 | 194 | 238 | 699467, | 12, 97, 172, | 1381771 | 686 |
| | | | 828423-828429 | 245, 318, 319, | | |
| | | | | 394, 395 | | |
| 8 | 236 | 268 | 828434-828438 | 26, 99, 174, | 1381772 | 687 |
| | | | | 320, 396 | | |
| 9 | 258 | 288 | 828440-828445 | 27, 100, 175, | 1381776 | 689 |
| | | | | 248, 321, 397 | | |
| 10 | 285 | 311 | 828447-828452 | 28, 101, 176, | 1381778 | 690 |
| | | | | 249, 323, 398 | | |
| 11 | 296 | 321 | 828454-828457 | 29, 102, 177, | n/a | n/a |
| | | | | 250 | | |
| 12 | 307 | 330 | 699500, 699501, | 13, 88, 163, | 1381789 | 692 |
| | | | 699503, 699505 | 386 | | |
| 13 | 330 | 352 | 699519, | 15, 103, 178, | n/a | n/a |
| | | | 828458-828461 | 251, 400 | | |
| 14 | 329 | 352 | 699518, 699519, | 15, 103, 178, | 1381790 | 693 |
| | | | 828458-828461 | 251, 313 400 | | |
| 15 | 339 | 383 | 828463-828467 | 30, 104, 179, | 1381798 | 697 |
| | | | | 252, 401 | | |
| 16 | 413 | 477 | 828480-828491 | 33, 34, 107, | 1381817, 1381818, | 702, 703, 704 |
| | | | | 108, 182, 183, | 1381825 | |
| | | | | 255, 256, 327, | | |
| | | | | 328, 404, 405 | | |
| 17 | 415 | 477 | 828480-828491 | 33, 34, 107, | 1381817, 1381825 | 702, 704 |
| | | | | 108, 182, 183, | | |
| | | | | 255, 256, 327, | | |
| | | | | 328, 404, 405 | | |
| 18 | 415 | 439 | 828480-828482 | 33, 327, 404 | n/a | n/a |
| 19 | 477 | 506 | 699533, 699535, | 16, 257, 330, | n/a | n/a |
| | | | 828497-828498, | 388, 475 ,502 | | |
| | | | 912249-912251, | | | |
| | | | 912292 | | | |
| 20 | 477 | 523 | 699533, 699535, | 16, 90, 165, | 1381832 | 708 |
| | | | 699537,699539, | 257, 258, 330, | | |
| | | | 828497-828499, | 388, 475-477, | | |
| | | | 912249-912255, | 502-504 | | |
| | | | 912292-912294 | | | |
| 21 | 477 | 541 | 699533, 699535, | 16, 36, 90, | 1381832, 1381904 | 708, 731 |
| | | | 699537, 699539, | 110, 165, 257, | | |
| | | | 828497-828503, | 258, 330, 331, | | |
| | | | 912249-912255, | 388, 407, 475- | | |
| | | | 912292-912294 | 477, 502-504 | | |
| 22 | 530 | 557 | 828507-828509 | 37, 111, 408 | n/a | n/a |
| 23 | 581 | 638 | 828526, 828527 | 40, 114 | 1381918, 1381923 | 736, 738 |
| 24 | 636 | 661 | 828531-828535 | 41, 115, 190, | 1381935 | 743 |
| | | | | 264, 412 | | |
| 25 | 652 | 697 | 828537-828547, | 42, 43, 116, | 1381953 | 748 |
| | | | 912256-912272, | 117, 191, 192, | | |
| | | | 912295-912297, | 265, 266, 338, | | |
| | | | 912303-912306 | 413,414, 478- | | |
| | | | | 487, 505-507, | | |
| | | | | 513-516 | | |
| 26 | 728 | 821 | 828550-828551 | 44, 118 | 1381982, 1381988, | 759, 760, 767, |
| | | | | | 1382012, 1382030 | 773 |
| 27 | 770 | 821 | 828551 | 118 | 1382012, 1382030 | 767,773 |
| 28 | 920 | 950 | 828769-878774 | 78, 154, 228, | 1382212 | 804 |
| | | | | 303, 376, 452 | | |
| 29 | 1006 | 1049 | 828790-828792 | 82, 157, 232 | 1382237, 1382243 | 807, 810 |
| 30 | 1152 | 1179 | 828565-828569 | 46, 120, 268, | n/a | n/a |
| | | | | 341, 417 | | |
| 31 | 1227 | 1274 | 699590,699592, | 17, 18, 48, 92, | 1381746 | 679 |
| | | | 699594,699596, | 122, 166, 270, | | |
| | | | 699600, | 271, 343, 344, | | |
| | | | 828577-828583 | 390, 419 | | |
| 32 | 1227 | 1265 | 699590, 699592, | 17, 48, 92, | n/a | n/a |
| | | | 699594, 699596, | 122, 166, 270, | | |
| | | | 828577-828583 | 271, 343, 344, | | |
| | | | | 390, 419 | | |
| 33 | 1268 | 1332 | 828584 | 197 | 1381751, 1381752, | 680, 681, 683 |
| | | | | | 1381756 | |
| 34 | 1268 | 1311 | n/a | n/a | 1381751, 1381752 | 680, 681 |
| 35 | 1289 | 1332 | 828584 | 197 | 1381752, 1381756 | 681, 683 |
| 36 | 1518 | 1543 | 828598-828602 | 51, 125, 200, | 1381828 | 706 |
| | | | | 274, 422 | | |
| 37 | 1531 | 1593 | 699631, | 19, 52, 53, | 1381835, 1381840 | 709, 710 |
| | | | 828604-828617 | 126-128, 201, | | |
| | | | | 202, 275, 276, | | |
| | | | | 349, 350, | | |
| | | | | 423-425 | | |
| 38 | 1544 | 1593 | 699631, | 19, 52, 53, | 1381840 | 710 |
| | | | 828605-828617 | 126-128, 201, | | |
| | | | | 202, 275, 276, | | |
| | | | | 349, 350, 424, | | |
| | | | | 425 | | |
| 39 | 1634 | 1657 | 828641-828643, | 207, 281, 354, | n/a | n/a |
| | | | 912285-912291, | 498-510 | | |
| | | | 912298-912300 | | | |
| 40 | 1778 | 1800 | 828656-828658 | 60, 135, 432 | n/a | n/a |
| 41 | 1882 | 1908 | 828674-828678 | 62, 138, 213, | n/a | n/a |
| | | | | 287, 360 | | |
| 42 | 2051 | 2074 | 828708-828710 | 68, 144, 441 | n/a | n/a |
| 43 | 2360 | 3117 | n/a | n/a | 1381981, 1381994, | 758, 761-766, |
| | | | | | 1381995, 1381998, | 769, 770, 772, |
| | | | | | 1381999, 1382004, | 774, 775, 777, |
| | | | | | 1382006, 1382019, | 779, 780-794, |
| | | | | | 1382020, 1382025, | 795-801 |
| | | | | | 1382033, 1382034, | |
| | | | | | 1382039, 1382051- | |
| | | | | | 1382054, 1382059, | |
| | | | | | 1382063, 1382069- | |
| | | | | | 1382071, 1382075, | |
| | | | | | 1382078, 1382080, | |
| | | | | | 1382087-1382090, | |
| | | | | | 1382103-1382107, | |
| | | | | | 1382116, 1382119 | |
| 44 | 2402 | 3117 | n/a | n/a | 1381995, 1381998, | 762-766, 769, |
| | | | | | 1381999, 1382004, | 770, 772, 774, |
| | | | | | 1382006, 1382019, | 775, 777, 779, |
| | | | | | 1382020, 1382025, | 780-794, |
| | | | | | 1382033, 1382034, | 795-801 |
| | | | | | 1382039, 1382051- | |
| | | | | | 1382054, 1382059, | |
| | | | | | 1382063, 1382069- | |
| | | | | | 1382071, 1382075, | |
| | | | | | 1382078, 1382080, | |
| | | | | | 1382087-1382090, | |
| | | | | | 1382103-1382107, | |
| | | | | | 1382116, 1382119 | |
| 45 | 2360 | 2655 | n/a | n/a | 1381981, 1381994, | 758, 761-766, |
| | | | | | 1381995, 1381998, | 769, 770, 772, |
| | | | | | 1381999, 1382004, | 774, 775, 777, |
| | | | | | 1382006, 1382019, | 779 |
| | | | | | 1382020, 1382025, | |
| | | | | | 1382033, 1382034, | |
| | | | | | 1382039, 1382051 | |
| 46 | 2402 | 2655 | n/a | n/a | 1381995, 1381998, | 762-766, 769, |
| | | | | | 1381999, 1382004, | 770, 772, 774, |
| | | | | | 1382006, 1382019, | 775, 777, 779 |
| | | | | | 1382020, 1382025, | |
| | | | | | 1382033, 1382034, | |
| | | | | | 1382039, 1382051 | |
| 47 | 2675 | 3054 | n/a | n/a | 1382052, 1382054, | 780, 782-794, |
| | | | | | 1382059, 1382063, | 795-797, 800 |
| | | | | | 1382069-1382071, | |
| | | | | | 1382075, 1382078, | |
| | | | | | 1382080, 1382087- | |
| | | | | | 1382090, 1382103- | |
| | | | | | 1382105, 1382116 | |

### Nonlimiting Disclosure and Incorporation by Reference

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein mC indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of Mixed Wing and Mixed Backbone Modified Oligonucleotides on Human APP RNA In Vitro, Single Dose

Modified oligonucleotides complementary to human APP nucleic acid were tested for their effect on APP RNA levels in vitro.

Modified oligonucleotides in the tables below are 18 nucleosides in length and have the sugar motif eeeeeddddddddkkeee, wherein each “e” is nucleoside comprising a 2′-MOE sugar moiety, each “k” is a nucleoside comprising a cEt sugar moiety, and each “d” is a nucleoside comprising a 2′-β-D-deoxyribosyl sugar moiety. The internucleoside linkage motif is sooosssssssssooss, wherein each “s” represents a phosphorothioate internucleoside linkage and each “o” represents a phosphodiester internucleoside linkage. All cytosine residues are 5-methylcytosines.

“Start site” indicates the 5′-most nucleoside to which the modified oligonucleotide is complementary in the human gene sequence. “Stop site” indicates the 3′-most nucleoside to which the modified oligonucleotide is complementary in the human gene sequence. Each modified oligonucleotide listed in the Tables below is 100% complementary to SEQ ID NO: 1 (the cDNA of Ensembl transcript ENST00000346798.7), the complement of SEQ ID NO: 2 (GENBANK Accession No. NC_000021.9 truncated from nucleotides 25878001 to 26174000), SEQ ID NO: 3 (the cDNA of Ensembl transcript ENST00000357903.7), SEQ ID NO: 4 (the cDNA of Ensembl transcript ENST00000348990.9), SEQ ID NO: 5 (the cDNA of Ensembl transcript ENST00000440126.7), SEQ ID NO: 6 (the cDNA of Ensembl transcript ENST00000354192.7), and/or SEQ ID NO: 7 (the cDNA of Ensembl transcript ENST00000358918.7). If a modified oligonucleotide is 100% complementary to SEQ ID NO: 1 and/or SEQ ID NO: 2, it may also be 100% complementary to any of SEQ ID NOs: 3-7, but this information is not displayed in the tables below. ‘N/A’ indicates that the modified oligonucleotide is not 100% complementary to that particular gene sequence.

Cultured SH-SY5Y cells at a density of 20,000 cells per well were treated with 7,000 nM of modified oligonucleotide by electroporation. After a treatment period of approximately 24 hours, total RNA was isolated from the cells and APP RNA levels were measured by quantitative real-time RTPCR. Human APP primer probe set HTS96 (forward sequence CCTTCCCGTGAATGGAGAGTT, designated herein as SEQ ID NO: 910; reverse sequence CACAGAGTCAGCCCCAAAAGA, designated herein as SEQ ID NO: 911; probe sequence CCTGGACGATCTCCAGCCGTGG, designated herein as SEQ ID NO: 912) was used to measure RNA levels. APP RNA levels were adjusted according to total RNA content, as measured by RIBOGREEN®. Results are presented in the tables below as percent APP RNA levels relative to untreated control cells.

TABLE 2
Reduction of APP RNA by 5-8-5 gapmers with mixed wings and a mixed backbone

| | SEQ ID | SEQ ID | SEQ ID | SEQ ID | | APP | |
|---|---|---|---|---|---|---|---|
| | NO: 1 | NO: 1 | NO: 2 | NO: 2 | | RNA | SEQ |
| Compound | Start | Stop | Start | Stop | | (% | ID |
| ID | Site | Site | Site | Site | Sequence (5’ to 3’) | control) | NO. |
| 699467 | 207 | 224 | 61970 | 61987 | CAATGCAGGTTTTGGTCC | 42 | 12 |
| 699501 | 308 | 325 | 83977 | 83994 | CCAGTTCTGGATGGTCAC | 40 | 13 |
| 699511 | 321 | 338 | 83990 | 84007 | GGCCCCGCTTGCACCAGT | 47 | 14 |
| 699519 | 331 | 348 | 84000 | 84017 | CACTGCTTGCGGCCCCGC | 36 | 15 |
| 699535 | 489 | 506 | N/A | N/A | ATGTCTCTTTGGCGACGG | 52 | 16 |
| 699594 | 1246 | 1263 | N/A | N/A | ATGACCTGGGACATTCTC | 26 | 17 |
| 699600 | 1257 | 1274 | 197971 | 197988 | CCCATTCTCTCATGACCT | 29 | 18 |
| 699631 | 1544 | 1561 | 218297 | 218314 | TAGGGTGTGCTGTCTGTC | 28 | 19 |
| 699660 | 1844 | 1861 | 262161 | 262178 | CACGGGAAGGAGCTCCAC | 69 | 20 |
| 828401 | 36 | 53 | 3382 | 3399 | GTGCCAAACCGGGCAGCA | 58 | 21 |
| 828407 | 61 | 78 | 3407 | 3424 | GCCGTCCAGGCGGCCAGC | 46 | 22 |
| 828413 | 83 | 100 | N/A | N/A | AGTGGGTACCTCCAGCGC | 54 | 23 |
| 828419 | 98 | 115 | 61861 | 61878 | GCCAGCATTACCATCAGT | 53 | 24 |
| 828430 | 224 | 241 | 61987 | 62004 | GATGCCTTCCTTGGTATC | 70 | 25 |
| 828436 | 246 | 263 | N/A | N/A | AGACTTCTTGGCAATACT | 24 | 26 |
| 828442 | 260 | 277 | 83929 | 83946 | CTGCAGTTCAGGGTAGAC | 28 | 27 |
| 828448 | 286 | 303 | 83955 | 83972 | TGGTTGGCTTCTACCACA | 54 | 28 |
| 828454 | 296 | 313 | 83965 | 83982 | GGTCACTGGTTGGTTGGC | 40 | 29 |
| 828464 | 341 | 358 | 84010 | 84027 | ATGGGTCTTGCACTGCTT | 19 | 30 |
| 828470 | 370 | 387 | 84039 | 84056 | AAGCAGCGGTAGGGAATC | 49 | 31 |
| 828476 | 379 | 396 | N/A | N/A | TCACCAACTAAGCAGCGG | 70 | 32 |
| 828482 | 422 | 439 | 120685 | 120702 | GAATTTGCACTTGTCAGG | 36 | 33 |
| 828488 | 451 | 468 | 120714 | 120731 | TCGCAAACATCCATCCTC | 32 | 34 |
| 828494 | 466 | 483 | 120729 | 120746 | CAGTGAAGATGAGTTTCG | 73 | 35 |
| 828502 | 516 | 533 | 122821 | 122838 | CATGCAAGTTGGTACTCT | 36 | 36 |
| 828508 | 533 | 550 | 122838 | 122855 | CAGCAACATGCCGTAGTC | 30 | 37 |
| 828514 | 549 | 566 | 122854 | 122871 | TGTCAATTCCGCAGGGCA | 60 | 38 |
| 828520 | 563 | 580 | 122868 | 122885 | TACCCCTCGGAACTTGTC | 38 | 39 |
| 828526 | 589 | 606 | 122894 | 122911 | TCAGCCAGTGGGCAACAC | 29 | 40 |
| 828532 | 638 | 655 | 122943 | 122960 | ATCCGAGTCATCCTCCTC | 19 | 41 |
| 828538 | 654 | 671 | 122959 | 122976 | CTCCGCCCCACCAGACAT | 46 | 42 |
| 828544 | 674 | 691 | 122979 | 122996 | ATCTGCATAGTCTGTGTC | 47 | 43 |
| 828550 | 752 | 769 | 152014 | 152031 | GTCATCATCGGCTTCTTC | 24 | 44 |
| 828562 | 1130 | 1147 | 191529 | 191546 | GGTACTGGCTGCTGTTGT | 26 | 45 |
| 828568 | 1159 | 1176 | 191558 | 191575 | GTCTCGAGATACTTGTCA | 42 | 46 |
| 828574 | 1186 | 1203 | 191585 | 191602 | TGGGCATGTTCATTCTCA | 15 | 47 |
| 828580 | 1233 | 1250 | 191632 | 191649 | TTCTCTCTCGGTGCTTGG | 31 | 48 |
| 828587 | 1453 | 1470 | 198892 | 198909 | GCGGTGATGTAGTTCTCC | 35 | 49 |
| 828593 | 1476 | 1493 | N/A | N/A | GCCGAGGAGGAACAGCCT | 46 | 50 |
| 828599 | 1520 | 1537 | 218273 | 218290 | TTCTGCGCGGACATACTT | 49 | 51 |
| 828610 | 1564 | 1581 | 218317 | 218334 | CGCACATGCTCGAAATGC | 20 | 52 |
| 828616 | 1575 | 1592 | 218328 | 218345 | GATCCACCATGCGCACAT | 48 | 53 |
| 828622 | 1586 | 1603 | 218339 | 218356 | GGCTTTCTTGGGATCCAC | 42 | 54 |
| 828628 | 1598 | 1615 | 218351 | 218368 | CCGGATCTGAGCGGCTTT | 46 | 55 |
| 828634 | 1605 | 1622 | 218358 | 218375 | CCTGGGACCGGATCTGAG | 37 | 56 |
| 828639 | 1629 | 1646 | 219319 | 219336 | AAATCACACGGAGGTGTG | 72 | 57 |
| 828645 | 1648 | 1665 | 219338 | 219355 | GACTGATTCATGCGCTCA | 13 | 58 |
| 828651 | 1765 | 1782 | 262082 | 262099 | TCACTAATCATGTTGGCC | 45 | 59 |
| 828657 | 1781 | 1798 | 262098 | 262115 | GTAACTGATCCTTGGTTC | 42 | 60 |
| 828663 | 1816 | 1833 | 262133 | 262150 | GTTTCGGTCAAAGATGGC | 44 | 61 |
| 828674 | 1882 | 1899 | 262199 | 262216 | TGCCACGGCTGGAGATCG | 57 | 62 |
| 828680 | 1947 | 1964 | 268927 | 268944 | GGCGGGCATCAACAGGCT | 99 | 63 |
| 828686 | 1970 | 1987 | 268950 | 268967 | GGTCAGTCCTCGGTCGGC | 106 | 64 |
| 828692 | 1979 | 1996 | 268959 | 268976 | TGGTCGAGTGGTCAGTCC | 71 | 65 |
| 828697 | 1988 | 2005 | N/A | N/A | CCCAGAACCTGGTCGAGT | 86 | 66 |
| 828703 | 2017 | 2034 | 276347 | 276364 | GAGATCTCCTCCGTCTTG | 57 | 67 |
| 828709 | 2053 | 2070 | 276383 | 276400 | GAGTCATGTCGGAATTCT | 42 | 68 |
| 828715 | 2070 | 2087 | 276400 | 276417 | GATGAACTTCATATCCTG | 70 | 69 |
| 828721 | 2128 | 2145 | 282162 | 282179 | CCAATGATTGCACCTTTG | 43 | 70 |
| 828727 | 2141 | 2158 | 282175 | 282192 | GCCCACCATGAGTCCAAT | 54 | 71 |
| 828733 | 2153 | 2170 | 282187 | 282204 | TATGACAACACCGCCCAC | 70 | 72 |
| 828739 | 2173 | 2190 | 282207 | 282224 | GTGATGACGATCACTGTC | 74 | 73 |
| 828745 | 2286 | 2303 | 292270 | 292287 | AGCCGTTCTGCTGCATCT | 79 | 74 |
| 828751 | 885 | 902 | N/A | N/A | CCTCTCGAACCACCTCTT | 65 | 75 |
| 828757 | 897 | 914 | N/A | N/A | GTTCAGAGCACACCTCTC | 35 | 76 |
| 828763 | 910 | 927 | 173829 | 173846 | CCCGTCTCGGCTTGTTCA | 46 | 77 |
| 828769 | 920 | 937 | 173839 | 173856 | TCGGCACGGCCCCGTCTC | 55 | 78 |
| 828775 | 934 | 951 | 173853 | 173870 | CGGGAGATCATTGCTCGG | 78 | 79 |
| 828781 | 946 | 963 | 173865 | 173882 | TCAAAGTACCAGCGGGAG | 58 | 80 |
| 828785 | 989 | 1006 | 173908 | 173925 | ACATCCGCCGTAAAAGAA | 69 | 81 |
| 828790 | 1015 | 1032 | 173934 | 173951 | GTGTCAAAGTTGTTCCGG | 46 | 82 |
| 828796 | 1038 | 1055 | 173957 | 173974 | ACACGGCCATGCAGTACT | 46 | 83 |
| 828802 | 1069 | 1086 | 176586 | 176603 | TTGAGTAAACTTTGGGAC | 69 | 84 |
| 828808 | 1094 | 1111 | 176611 | 176628 | TCGGGCAAGAGGTTCCTG | 77 | 85 |
| 828814 | 1102 | 1119 | 176619 | 176636 | ACAGGATCTCGGGCAAGA | 42 | 86 |
| 828820 | N/A | N/A | 33811 | 33828 | ACAAGTCCTCTAATTGGT | 56 | 87 |

TABLE 3
Reduction of APP RNA by 5-8-5 gapmers with mixed wings and a mixed backbone

| | SEQ ID | SEQ ID | SEQ ID | SEQ ID | | APP | |
|---|---|---|---|---|---|---|---|
| | NO: 1 | NO: 1 | NO: 2 | NO: 2 | | RNA | SEQ |
| Compound | Start | Stop | Start | Stop | | (% | ID |
| ID | Site | Site | Site | Site | Sequence (5’ to 3’) | control) | NO. |
| 699503 | 311 | 328 | 83980 | 83997 | GCACCAGTTCTGGATGGT | 49 | 88 |
| 699512 | 323 | 340 | 83992 | 84009 | GCGGCCCCGCTTGCACCA | 77 | 89 |
| 699537 | 491 | 508 | N/A | N/A | GCATGTCTCTTTGGCGAC | 72 | 90 |
| 699568 | 948 | 965 | 173867 | 173884 | CATCAAAGTACCAGCGGG | 70 | 91 |
| 699596 | 1248 | 1265 | N/A | N/A | TCATGACCTGGGACATTC | 48 | 92 |
| 699600 | 1257 | 1274 | 197971 | 197988 | CCCATTCTCTCATGACCT | 34 | 18 |
| 828402 | 38 | 55 | 3384 | 3401 | CAGTGCCAAACCGGGCAG | 69 | 93 |
| 828408 | 63 | 80 | 3409 | 3426 | GAGCCGTCCAGGCGGCCA | 80 | 94 |
| 828414 | 84 | 101 | N/A | N/A | CAGTGGGTACCTCCAGCG | 59 | 95 |
| 828420 | 112 | 129 | 61875 | 61892 | GGTTCAGCCAGCAGGCCA | 39 | 96 |
| 828425 | 212 | 229 | 61975 | 61992 | GGTATCAATGCAGGTTTT | 25 | 97 |
| 828431 | 225 | 242 | 61988 | 62005 | GGATGCCTTCCTTGGTAT | 63 | 98 |
| 828437 | 250 | 267 | N/A | N/A | GGGTAGACTTCTTGGCAA | 38 | 99 |
| 828443 | 265 | 282 | 83934 | 83951 | GTGATCTGCAGTTCAGGG | 22 | 100 |
| 828449 | 288 | 305 | 83957 | 83974 | GTTGGTTGGCTTCTACCA | 47 | 101 |
| 828455 | 300 | 317 | 83969 | 83986 | GGATGGTCACTGGTTGGT | 24 | 102 |
| 828459 | 332 | 349 | 84001 | 84018 | GCACTGCTTGCGGCCCCG | 36 | 103 |
| 828465 | 343 | 360 | 84012 | 84029 | GGATGGGTCTTGCACTGC | 39 | 104 |
| 828471 | 372 | 389 | 84041 | 84058 | CTAAGCAGCGGTAGGGAA | 70 | 105 |
| 828477 | 381 | 398 | N/A | N/A | ACTCACCAACTAAGCAGC | 52 | 106 |
| 828483 | 429 | 446 | 120692 | 120709 | GGTGTAAGAATTTGCACT | 61 | 107 |
| 828489 | 453 | 470 | 120716 | 120733 | TTTCGCAAACATCCATCC | 49 | 108 |
| 828495 | 473 | 490 | 120736 | 120753 | GGTGTGCCAGTGAAGATG | 64 | 109 |
| 828503 | 524 | 541 | 122829 | 122846 | GCCGTAGTCATGCAAGTT | 37 | 110 |
| 828509 | 540 | 557 | 122845 | 122862 | CGCAGGGCAGCAACATGC | 31 | 111 |
| 828515 | 551 | 568 | 122856 | 122873 | CTTGTCAATTCCGCAGGG | 74 | 112 |
| 828521 | 564 | 581 | 122869 | 122886 | CTACCCCTCGGAACTTGT | 67 | 113 |
| 828527 | 621 | 638 | 122926 | 122943 | CCGCATCAGCAGAATCCA | 24 | 114 |
| 828533 | 639 | 656 | 122944 | 122961 | CATCCGAGTCATCCTCCT | 16 | 115 |
| 828539 | 657 | 674 | 122962 | 122979 | CTGCTCCGCCCCACCAGA | 47 | 116 |
| 828545 | 676 | 693 | 122981 | 122998 | CCATCTGCATAGTCTGTG | 24 | 117 |
| 828551 | 804 | 821 | 152066 | 152083 | CGTAGGGTTCCTCAGCCT | 42 | 118 |
| 828563 | 1131 | 1148 | 191530 | 191547 | GGGTACTGGCTGCTGTTG | 41 | 119 |
| 828569 | 1162 | 1179 | 191561 | 191578 | GGTGTCTCGAGATACTTG | 52 | 120 |
| 828575 | 1224 | 1241 | 191623 | 191640 | GGTGCTTGGCCTCAAGCC | 71 | 121 |
| 828581 | 1235 | 1252 | 191634 | 191651 | CATTCTCTCTCGGTGCTT | 67 | 122 |
| 828588 | 1455 | 1472 | 198894 | 198911 | GAGCGGTGATGTAGTTCT | 65 | 123 |
| 828594 | 1485 | 1502 | N/A | N/A | CGTGACGAGGCCGAGGAG | 88 | 124 |
| 828600 | 1521 | 1538 | 218274 | 218291 | GTTCTGCGCGGACATACT | 35 | 125 |
| 828605 | 1546 | 1563 | 218299 | 218316 | TTTAGGGTGTGCTGTCTG | 29 | 126 |
| 828611 | 1566 | 1583 | 218319 | 218336 | TGCGCACATGCTCGAAAT | 52 | 127 |
| 828617 | 1576 | 1593 | 218329 | 218346 | GGATCCACCATGCGCACA | 46 | 128 |
| 828623 | 1588 | 1605 | 218341 | 218358 | GCGGCTTTCTTGGGATCC | 60 | 129 |
| 828629 | 1599 | 1616 | 218352 | 218369 | ACCGGATCTGAGCGGCTT | 50 | 130 |
| 828635 | 1607 | 1624 | N/A | N/A | AACCTGGGACCGGATCTG | 69 | 131 |
| 828640 | 1632 | 1649 | 219322 | 219339 | CATAAATCACACGGAGGT | 61 | 132 |
| 828646 | 1650 | 1667 | 219340 | 219357 | GAGACTGATTCATGCGCT | 30 | 133 |
| 828652 | 1768 | 1785 | 262085 | 262102 | GGTTCACTAATCATGTTG | 50 | 134 |
| 828658 | 1783 | 1800 | 262100 | 262117 | CCGTAACTGATCCTTGGT | 43 | 135 |
| 828664 | 1833 | 1850 | 262150 | 262167 | GCTCCACGGTGGTTTTCG | 69 | 136 |
| 828669 | 1848 | 1865 | 262165 | 262182 | CATTCACGGGAAGGAGCT | 39 | 137 |
| 828675 | 1883 | 1900 | 262200 | 262217 | ATGCCACGGCTGGAGATC | 43 | 138 |
| 828681 | 1961 | 1978 | 268941 | 268958 | TCGGTCGGCAGCAGGGCG | 98 | 139 |
| 828687 | 1971 | 1988 | 268951 | 268968 | TGGTCAGTCCTCGGTCGG | 87 | 140 |
| 828693 | 1981 | 1998 | 268961 | 268978 | CCTGGTCGAGTGGTCAGT | 91 | 141 |
| 828698 | 1991 | 2008 | N/A | N/A | CAACCCAGAACCTGGTCG | 88 | 142 |
| 828704 | 2019 | 2036 | 276349 | 276366 | CAGAGATCTCCTCCGTCT | 46 | 143 |
| 828710 | 2057 | 2074 | 276387 | 276404 | TCCTGAGTCATGTCGGAA | 49 | 144 |
| 828716 | 2073 | 2090 | 276403 | 276420 | GATGATGAACTTCATATC | 80 | 145 |
| 828722 | 2130 | 2147 | 282164 | 282181 | GTCCAATGATTGCACCTT | 51 | 146 |
| 828728 | 2143 | 2160 | 282177 | 282194 | CCGCCCACCATGAGTCCA | 91 | 147 |
| 828734 | 2155 | 2172 | 282189 | 282206 | GCTATGACAACACCGCCC | 41 | 148 |
| 828740 | 2175 | 2192 | 282209 | 282226 | AGGTGATGACGATCACTG | 75 | 149 |
| 828746 | 2288 | 2305 | 292272 | 292289 | GTAGCCGTTCTGCTGCAT | 85 | 150 |
| 828752 | 887 | 904 | N/A | N/A | CACCTCTCGAACCACCTC | 49 | 151 |
| 828758 | 900 | 917 | 173819 | 173836 | CTTGTTCAGAGCACACCT | 55 | 152 |
| 828764 | 912 | 929 | 173831 | 173848 | GCCCCGTCTCGGCTTGTT | 42 | 153 |
| 828770 | 924 | 941 | 173843 | 173860 | TTGCTCGGCACGGCCCCG | 33 | 154 |
| 828776 | 935 | 952 | 173854 | 173871 | GCGGGAGATCATTGCTCG | 53 | 155 |
| 828786 | 992 | 1009 | 173911 | 173928 | GCCACATCCGCCGTAAAA | 72 | 156 |
| 828791 | 1017 | 1034 | 173936 | 173953 | CTGTGTCAAAGTTGTTCC | 38 | 157 |
| 828797 | 1039 | 1056 | 173958 | 173975 | CACACGGCCATGCAGTAC | 38 | 158 |
| 828803 | 1077 | 1094 | 176594 | 176611 | GGGTAGTCTTGAGTAAAC | 54 | 159 |
| 828809 | 1095 | 1112 | 176612 | 176629 | CTCGGGCAAGAGGTTCCT | 90 | 160 |
| 828815 | 1105 | 1122 | 176622 | 176639 | TTAACAGGATCTCGGGCA | 58 | 161 |
| 828821 | N/A | N/A | 33815 | 33832 | ACCAACAAGTCCTCTAAT | 105 | 162 |

TABLE 4
Reduction of APP RNA by 5-8-5 gapmers with mixed wings and a mixed backbone

| | SEQ ID | SEQ ID | SEQ ID | SEQ ID | | APP | |
|---|---|---|---|---|---|---|---|
| | NO: 1 | NO: 1 | NO: 2 | NO: 2 | | RNA | SEQ |
| Compound | Start | Stop | Start | Stop | | (% | ID |
| ID | Site | Site | Site | Site | Sequence (5’ to 3’) | control) | NO: |
| 699505 | 313 | 330 | 83982 | 83999 | TTGCACCAGTTCTGGATG | 53 | 163 |
| 699514 | 325 | 342 | 83994 | 84011 | TTGCGGCCCCGCTTGCAC | 61 | 164 |
| 699539 | 493 | 510 | N/A | N/A | CTGCATGTCTCTTTGGCG | 42 | 165 |
| 699590 | 1237 | 1254 | 191636 | 191653 | GACATTCTCTCTCGGTGC | 66 | 166 |
| 699600 | 1257 | 1274 | 197971 | 197988 | CCCATTCTCTCATGACCT | 60 | 18 |
| 699669 | 1983 | 2000 | N/A | N/A | AACCTGGTCGAGTGGTCA | 166 | 167 |
| 828403 | 39 | 56 | 3385 | 3402 | GCAGTGCCAAACCGGGCA | 73 | 168 |
| 828409 | 66 | 83 | 3412 | 3429 | CCCGAGCCGTCCAGGCGG | 70 | 169 |
| 828415 | 86 | 103 | N/A | N/A | ATCAGTGGGTACCTCCAG | 56 | 170 |
| 828421 | 129 | 146 | 61892 | 61909 | AGAACATGGCAATCTGGG | 45 | 171 |
| 828426 | 214 | 231 | 61977 | 61994 | TTGGTATCAATGCAGGTT | 37 | 172 |
| 828432 | 233 | 250 | 61996 | 62013 | ATACTGCAGGATGCCTTC | 108 | 173 |
| 828438 | 251 | 268 | N/A | N/A | AGGGTAGACTTCTTGGCA | 54 | 174 |
| 828444 | 266 | 283 | 83935 | 83952 | GGTGATCTGCAGTTCAGG | 19 | 175 |
| 828450 | 289 | 306 | 83958 | 83975 | GGTTGGTTGGCTTCTACC | 52 | 176 |
| 828456 | 302 | 319 | 83971 | 83988 | CTGGATGGTCACTGGTTG | 38 | 177 |
| 828460 | 334 | 351 | 84003 | 84020 | TTGCACTGCTTGCGGCCC | 40 | 178 |
| 828466 | 361 | 378 | 84030 | 84047 | TAGGGAATCACAAAGTGG | 62 | 179 |
| 828472 | 373 | 390 | N/A | N/A | ACTAAGCAGCGGTAGGGA | 63 | 180 |
| 828478 | 409 | 426 | 120672 | 120689 | TCAGGAACGAGAAGGGCA | 67 | 181 |
| 828484 | 432 | 449 | 120695 | 120712 | CCTGGTGTAAGAATTTGC | 38 | 182 |
| 828490 | 456 | 473 | 120719 | 120736 | GAGTTTCGCAAACATCCA | 26 | 183 |
| 828496 | 474 | 491 | 120737 | 120754 | CGGTGTGCCAGTGAAGAT | 98 | 184 |
| 828504 | 525 | 542 | 122830 | 122847 | TGCCGTAGTCATGCAAGT | 82 | 185 |
| 828510 | 541 | 558 | 122846 | 122863 | CCGCAGGGCAGCAACATG | 76 | 186 |
| 828516 | 555 | 572 | 122860 | 122877 | GGAACTTGTCAATTCCGC | 115 | 187 |
| 828522 | 567 | 584 | 122872 | 122889 | ACTCTACCCCTCGGAACT | 101 | 188 |
| 828528 | 624 | 641 | 122929 | 122946 | CCTCCGCATCAGCAGAAT | 24 | 189 |
| 828534 | 643 | 660 | 122948 | 122965 | CAGACATCCGAGTCATCC | 45 | 190 |
| 828540 | 662 | 679 | 122967 | 122984 | TGTGTCTGCTCCGCCCCA | 49 | 191 |
| 828546 | 677 | 694 | 122982 | 122999 | CCCATCTGCATAGTCTGT | 33 | 192 |
| 828552 | 881 | 898 | 152143 | 152160 | TCGAACCACCTCTTCCAC | 88 | 193 |
| 828564 | 1147 | 1164 | 191546 | 191563 | TTGTCAACGGCATCAGGG | 88 | 194 |
| 828570 | 1164 | 1181 | 191563 | 191580 | CAGGTGTCTCGAGATACT | 80 | 195 |
| 828576 | 1225 | 1242 | 191624 | 191641 | CGGTGCTTGGCCTCAAGC | 88 | 196 |
| 828584 | 1315 | 1332 | 198029 | 198046 | TGGATAACTGCCTTCTTA | 150 | 197 |
| 828589 | 1457 | 1474 | 198896 | 198913 | CAGAGCGGTGATGTAGTT | 74 | 198 |
| 828595 | 1512 | 1529 | 218265 | 218282 | GGACATACTTCTTTAGCA | 67 | 199 |
| 828601 | 1524 | 1541 | 218277 | 218294 | TCTGTTCTGCGCGGACAT | 55 | 200 |
| 828606 | 1548 | 1565 | 218301 | 218318 | GCTTTAGGGTGTGCTGTC | 36 | 201 |
| 828612 | 1568 | 1585 | 218321 | 218338 | CATGCGCACATGCTCGAA | 48 | 202 |
| 828618 | 1577 | 1594 | 218330 | 218347 | GGGATCCACCATGCGCAC | 68 | 203 |
| 828624 | 1590 | 1607 | 218343 | 218360 | GAGCGGCTTTCTTGGGAT | 106 | 204 |
| 828630 | 1600 | 1617 | 218353 | 218370 | GACCGGATCTGAGCGGCT | 75 | 205 |
| 828636 | 1608 | 1625 | N/A | N/A | TAACCTGGGACCGGATCT | 105 | 206 |
| 828641 | 1635 | 1652 | 219325 | 219342 | GCTCATAAATCACACGGA | 40 | 207 |
| 828647 | 1684 | 1701 | 219374 | 219391 | TCGGCCACTGCAGGCACG | 87 | 208 |
| 828653 | 1771 | 1788 | 262088 | 262105 | CTTGGTTCACTAATCATG | 70 | 209 |
| 828659 | 1784 | 1801 | 262101 | 262118 | TCCGTAACTGATCCTTGG | 78 | 210 |
| 828665 | 1837 | 1854 | 262154 | 262171 | AGGAGCTCCACGGTGGTT | 165 | 211 |
| 828670 | 1849 | 1866 | 262166 | 262183 | CCATTCACGGGAAGGAGC | 31 | 212 |
| 828676 | 1885 | 1902 | 262202 | 262219 | GAATGCCACGGCTGGAGA | 17 | 213 |
| 828682 | 1963 | 1980 | 268943 | 268960 | CCTCGGTCGGCAGCAGGG | 151 | 214 |
| 828688 | 1973 | 1990 | 268953 | 268970 | AGTGGTCAGTCCTCGGTC | 97 | 215 |
| 828699 | 2001 | 2018 | 276331 | 276348 | TGATATTTGTCAACCCAG | 51 | 216 |
| 828705 | 2021 | 2038 | 276351 | 276368 | TTCAGAGATCTCCTCCGT | 101 | 217 |
| 828711 | 2058 | 2075 | 276388 | 276405 | ATCCTGAGTCATGTCGGA | 88 | 218 |
| 828717 | 2117 | 2134 | 282151 | 282168 | ACCTTTGTTTGAACCCAC | 47 | 219 |
| 828723 | 2132 | 2149 | 282166 | 282183 | GAGTCCAATGATTGCACC | 92 | 220 |
| 828729 | 2146 | 2163 | 282180 | 282197 | ACACCGCCCACCATGAGT | 94 | 221 |
| 828735 | 2157 | 2174 | 282191 | 282208 | TCGCTATGACAACACCGC | 47 | 222 |
| 828741 | 2209 | 2226 | 282243 | 282260 | ATGGATGTGTACTGTTTC | 46 | 223 |
| 828747 | 2289 | 2306 | 292273 | 292290 | CGTAGCCGTTCTGCTGCA | 157 | 224 |
| 828753 | 889 | 906 | N/A | N/A | CACACCTCTCGAACCACC | 56 | 225 |
| 828759 | 901 | 918 | 173820 | 173837 | GCTTGTTCAGAGCACACC | 63 | 226 |
| 828765 | 914 | 931 | 173833 | 173850 | CGGCCCCGTCTCGGCTTG | 119 | 227 |
| 828771 | 926 | 943 | 173845 | 173862 | CATTGCTCGGCACGGCCC | 59 | 228 |
| 828777 | 937 | 954 | 173856 | 173873 | CAGCGGGAGATCATTGCT | 75 | 229 |
| 828782 | 952 | 969 | 173871 | 173888 | GTCACATCAAAGTACCAG | 53 | 230 |
| 828787 | 993 | 1010 | 173912 | 173929 | CGCCACATCCGCCGTAAA | 100 | 231 |
| 828792 | 1032 | 1049 | 173951 | 173968 | CCATGCAGTACTCTTCTG | 87 | 232 |
| 828798 | 1041 | 1058 | 173960 | 173977 | CACACACGGCCATGCAGT | 80 | 233 |
| 828804 | 1080 | 1097 | 176597 | 176614 | CCTGGGTAGTCTTGAGTA | 114 | 234 |
| 828810 | 1097 | 1114 | 176614 | 176631 | ATCTCGGGCAAGAGGTTC | 69 | 235 |
| 828816 | 1108 | 1125 | N/A | N/A | AGTTTAACAGGATCTCGG | 71 | 236 |

TABLE 5
Reduction of APP RNA by 5-8-5 gapmers with mixed wings and a mixed backbone

| | SEQ ID | SEQ ID | SEQ ID | SEQ ID | | APP | |
|---|---|---|---|---|---|---|---|
| | NO: 1 | NO: 1 | NO: 2 | NO: 2 | | RNA | SEQ |
| Compound | Start | Stop | Start | Stop | | (% | ID |
| ID | Site | Site | Site | Site | Sequence (5′ to 3′) | control) | NO. |
| 699506 | 314 | 331 | 83983 | 84000 | CTTGCACCAGTTCTGGAT | 60 | 237 |
| 699516 | 327 | 344 | 83996 | 84013 | GCTTGCGGCCCCGCTTGC | 85 | 238 |
| 699573 | 995 | 1012 | 173914 | 173931 | GCCGCCACATCCGCCGTA | 97 | 239 |
| 699600 | 1257 | 1274 | 197971 | 197988 | CCCATTCTCTCATGACCT | 57 | 18 |
| 699623 | 1428 | 1445 | 198867 | 198884 | GGCGGCGGCGGTCATTGA | 105 | 240 |
| 828404 | 40 | 57 | 3386 | 3403 | AGCAGTGCCAAACCGGGC | 26 | 241 |
| 828410 | 67 | 84 | 3413 | 3430 | GCCCGAGCCGTCCAGGCG | 66 | 242 |
| 828416 | 89 | 106 | N/A | N/A | ACCATCAGTGGGTACCTC | 46 | 243 |
| 828422 | 150 | 167 | 61913 | 61930 | TGTGCATGTTCAGTCTGC | 23 | 244 |
| 828427 | 216 | 233 | 61979 | 61996 | CCTTGGTATCAATGCAGG | 45 | 245 |
| 828433 | 235 | 252 | 61998 | 62015 | CAATACTGCAGGATGCCT | 80 | 246 |
| 828439 | 252 | 269 | N/A | N/A | CAGGGTAGACTTCTTGGC | 104 | 247 |
| 828445 | 267 | 284 | 83936 | 83953 | TGGTGATCTGCAGTTCAG | 31 | 248 |
| 828451 | 291 | 308 | 83960 | 83977 | CTGGTTGGTTGGCTTCTA | 48 | 249 |
| 828457 | 304 | 321 | 83973 | 83990 | TTCTGGATGGTCACTGGT | 54 | 250 |
| 828461 | 335 | 352 | 84004 | 84021 | CTTGCACTGCTTGCGGCC | 48 | 251 |
| 828467 | 366 | 383 | 84035 | 84052 | AGCGGTAGGGAATCACAA | 58 | 252 |
| 828473 | 375 | 392 | N/A | N/A | CAACTAAGCAGCGGTAGG | 61 | 253 |
| 828479 | 410 | 427 | 120673 | 120690 | GTCAGGAACGAGAAGGGC | 117 | 254 |
| 828485 | 437 | 454 | 120700 | 120717 | CCTCTCCTGGTGTAAGAA | 42 | 255 |
| 828491 | 460 | 477 | 120723 | 120740 | AGATGAGTTTCGCAAACA | 52 | 256 |
| 828497 | 477 | 494 | 120740 | 120757 | CGACGGTGTGCCAGTGAA | 35 | 257 |
| 828499 | 506 | 523 | 122811 | 122828 | GGTACTCTTCTCACTGCA | 32 | 258 |
| 828505 | 527 | 544 | 122832 | 122849 | CATGCCGTAGTCATGCAA | 66 | 259 |
| 828511 | 543 | 560 | 122848 | 122865 | TTCCGCAGGGCAGCAACA | 57 | 260 |
| 828517 | 557 | 574 | 122862 | 122879 | TCGGAACTTGTCAATTCC | 82 | 261 |
| 828523 | 568 | 585 | 122873 | 122890 | AACTCTACCCCTCGGAAC | 79 | 262 |
| 828529 | 633 | 650 | 122938 | 122955 | AGTCATCCTCCTCCGCAT | 81 | 263 |
| 828535 | 644 | 661 | 122949 | 122966 | CCAGACATCCGAGTCATC | 40 | 264 |
| 828541 | 664 | 681 | 122969 | 122986 | TCTGTGTCTGCTCCGCCC | 33 | 265 |
| 828547 | 680 | 697 | N/A | N/A | ACTCCCATCTGCATAGTC | 43 | 266 |
| 828553 | 882 | 899 | 152144 | 152161 | CTCGAACCACCTCTTCCA | 100 | 267 |
| 828565 | 1152 | 1169 | 191551 | 191568 | GATACTTGTCAACGGCAT | 22 | 268 |
| 828571 | 1167 | 1184 | 191566 | 191583 | CCCCAGGTGTCTCGAGAT | 50 | 269 |
| 828577 | 1227 | 1244 | 191626 | 191643 | CTCGGTGCTTGGCCTCAA | 44 | 270 |
| 828582 | 1238 | 1255 | 191637 | 191654 | GGACATTCTCTCTCGGTG | 40 | 271 |
| 828590 | 1461 | 1478 | 198900 | 198917 | CCTGCAGAGCGGTGATGT | 98 | 272 |
| 828596 | 1515 | 1532 | 218268 | 218285 | CGCGGACATACTTCTTTA | 54 | 273 |
| 828602 | 1526 | 1543 | 218279 | 218296 | CTTCTGTTCTGCGCGGAC | 51 | 274 |
| 828607 | 1550 | 1567 | 218303 | 218320 | ATGCTTTAGGGTGTGCTG | 66 | 275 |
| 828613 | 1569 | 1586 | 218322 | 218339 | CCATGCGCACATGCTCGA | 67 | 276 |
| 828619 | 1579 | 1596 | 218332 | 218349 | TTGGGATCCACCATGCGC | 64 | 277 |
| 828625 | 1592 | 1609 | 218345 | 218362 | CTGAGCGGCTTTCTTGGG | 84 | 278 |
| 828631 | 1601 | 1618 | 218354 | 218371 | GGACCGGATCTGAGCGGC | 62 | 279 |
| 828637 | 1610 | 1627 | N/A | N/A | CATAACCTGGGACCGGAT | 38 | 280 |
| 828642 | 1637 | 1654 | 219327 | 219344 | GCGCTCATAAATCACACG | 55 | 281 |
| 828648 | 1692 | 1709 | 219382 | 219399 | GAATCTCCTCGGCCACTG | 38 | 282 |
| 828654 | 1773 | 1790 | 262090 | 262107 | TCCTTGGTTCACTAATCA | 67 | 283 |
| 828660 | 1788 | 1805 | 262105 | 262122 | CGTTTCCGTAACTGATCC | 83 | 284 |
| 828666 | 1839 | 1856 | 262156 | 262173 | GAAGGAGCTCCACGGTGG | 139 | 285 |
| 828671 | 1851 | 1868 | 262168 | 262185 | CTCCATTCACGGGAAGGA | 35 | 286 |
| 828677 | 1887 | 1904 | 262204 | 262221 | AAGAATGCCACGGCTGGA | 25 | 287 |
| 828683 | 1965 | 1982 | 268945 | 268962 | GTCCTCGGTCGGCAGCAG | 198 | 288 |
| 828689 | 1975 | 1992 | 268955 | 268972 | CGAGTGGTCAGTCCTCGG | 130 | 289 |
| 828694 | 1984 | 2001 | N/A | N/A | GAACCTGGTCGAGTGGTC | 131 | 290 |
| 828700 | 2010 | 2027 | 276340 | 276357 | CCTCCGTCTTGATATTTG | 291 | 291 |
| 828706 | 2046 | 2063 | 276376 | 276393 | GTCGGAATTCTGCATCCA | 50 | 292 |
| 828712 | 2059 | 2076 | 276389 | 276406 | TATCCTGAGTCATGTCGG | 84 | 293 |
| 828718 | 2119 | 2136 | 282153 | 282170 | GCACCTTTGTTTGAACCC | 48 | 294 |
| 828724 | 2134 | 2151 | 282168 | 282185 | ATGAGTCCAATGATTGCA | 55 | 295 |
| 828730 | 2147 | 2164 | 282181 | 282198 | AACACCGCCCACCATGAG | 113 | 296 |
| 828736 | 2162 | 2179 | 282196 | 282213 | CACTGTCGCTATGACAAC | 139 | 297 |
| 828742 | 2223 | 2240 | 282257 | 282274 | CCACACCATGATGAATGG | 84 | 298 |
| 828748 | 2305 | 2322 | 292289 | 292306 | TTGTAGGTTGGATTTTCG | 76 | 299 |
| 828754 | 890 | 907 | N/A | N/A | GCACACCTCTCGAACCAC | 71 | 300 |
| 828760 | 904 | 921 | 173823 | 173840 | TCGGCTTGTTCAGAGCAC | 90 | 301 |
| 828766 | 916 | 933 | 173835 | 173852 | CACGGCCCCGTCTCGGCT | 71 | 302 |
| 828772 | 928 | 945 | 173847 | 173864 | ATCATTGCTCGGCACGGC | 45 | 303 |
| 828778 | 940 | 957 | 173859 | 173876 | TACCAGCGGGAGATCATT | 68 | 304 |
| 828783 | 954 | 971 | 173873 | 173890 | CAGTCACATCAAAGTACC | 33 | 305 |
| 828793 | 1034 | 1051 | 173953 | 173970 | GGCCATGCAGTACTCTTC | 90 | 306 |
| 828799 | 1047 | 1064 | 173966 | 173983 | CGCTGCCACACACGGCCA | 73 | 307 |
| 828805 | 1081 | 1098 | 176598 | 176615 | TCCTGGGTAGTCTTGAGT | 124 | 308 |
| 828811 | 1098 | 1115 | 176615 | 176632 | GATCTCGGGCAAGAGGTT | 74 | 309 |
| 828817 | 1111 | 1128 | N/A | N/A | GGAAGTTTAACAGGATCT | 80 | 310 |

TABLE 6
Reduction of APP RNA by 5-8-5 gapmers with mixed wings and a mixed backbone

| | SEQ ID | SEQ ID | SEQ ID | SEQ ID | | APP | |
|---|---|---|---|---|---|---|---|
| | NO: 1 | NO: 1 | NO: 2 | NO: 2 | | RNA | SEQ |
| Compound | Start | Stop | Start | Stop | | (% | ID |
| ID | Site | Site | Site | Site | Sequence (5′ to 3′) | control) | NO |
| 699498 | 305 | 322 | 83974 | 83991 | GTTCTGGATGGTCACTGG | 83 | 311 |
| 699508 | 316 | 333 | 83985 | 84002 | CGCTTGCACCAGTTCTGG | 65 | 312 |
| 699518 | 329 | 346 | 83998 | 84015 | CTGCTTGCGGCCCCGCTT | 67 | 313 |
| 699600 | 1257 | 1274 | 197971 | 197988 | CCCATTCTCTCATGACCT | 61 | 18 |
| 699644 | 1612 | 1629 | N/A | N/A | GTCATAACCTGGGACCGG | 77 | 314 |
| 828405 | 42 | 59 | 3388 | 3405 | GGAGCAGTGCCAAACCGG | 44 | 315 |
| 828411 | 68 | 85 | 3414 | 3431 | CGCCCGAGCCGTCCAGGC | 81 | 316 |
| 828417 | 94 | 111 | 61857 | 61874 | GCATTACCATCAGTGGGT | 39 | 317 |
| 828423 | 194 | 211 | 61957 | 61974 | GGTCCCTGATGGATCTGA | 50 | 318 |
| 828428 | 219 | 236 | 61982 | 61999 | CTTCCTTGGTATCAATGC | 29 | 319 |
| 828434 | 236 | 253 | 61999 | 62016 | GCAATACTGCAGGATGCC | 48 | 320 |
| 828440 | 258 | 275 | N/A | N/A | GCAGTTCAGGGTAGACTT | 51 | 321 |
| 828446 | 279 | 296 | 83948 | 83965 | CTTCTACCACATTGGTGA | 88 | 322 |
| 828452 | 294 | 311 | 83963 | 83980 | TCACTGGTTGGTTGGCTT | 41 | 323 |
| 828462 | 337 | 354 | 84006 | 84023 | GTCTTGCACTGCTTGCGG | 84 | 324 |
| 828468 | 367 | 384 | 84036 | 84053 | CAGCGGTAGGGAATCACA | 71 | 325 |
| 828474 | 376 | 393 | N/A | N/A | CCAACTAAGCAGCGGTAG | 76 | 326 |
| 828480 | 415 | 432 | 120678 | 120695 | CACTTGTCAGGAACGAGA | 35 | 327 |
| 828486 | 439 | 456 | 120702 | 120719 | ATCCTCTCCTGGTGTAAG | 77 | 328 |
| 828492 | 462 | 479 | 120725 | 120742 | GAAGATGAGTTTCGCAAA | 80 | 329 |
| 828498 | 478 | 495 | 120741 | 120758 | GCGACGGTGTGCCAGTGA | 64 | 330 |
| 828500 | 509 | 526 | 122814 | 122831 | GTTGGTACTCTTCTCACT | 64 | 331 |
| 828506 | 528 | 545 | 122833 | 122850 | ACATGCCGTAGTCATGCA | 114 | 332 |
| 828512 | 545 | 562 | 122850 | 122867 | AATTCCGCAGGGCAGCAA | 63 | 333 |
| 828518 | 561 | 578 | 122866 | 122883 | CCCCTCGGAACTTGTCAA | 96 | 334 |
| 828524 | 570 | 587 | 122875 | 122892 | CAAACTCTACCCCTCGGA | 75 | 335 |
| 828530 | 634 | 651 | 122939 | 122956 | GAGTCATCCTCCTCCGCA | 70 | 336 |
| 828536 | 646 | 663 | 122951 | 122968 | CACCAGACATCCGAGTCA | 96 | 337 |
| 828542 | 666 | 683 | 122971 | 122988 | AGTCTGTGTCTGCTCCGC | 26 | 338 |
| 828548 | 681 | 698 | N/A | N/A | CACTCCCATCTGCATAGT | 71 | 339 |
| 828560 | N/A | N/A | 191523 | 191540 | GGCTGCTGTTGTAGGAAC | 45 | 340 |
| 828566 | 1154 | 1171 | 191553 | 191570 | GAGATACTTGTCAACGGC | 41 | 341 |
| 828572 | 1168 | 1185 | 191567 | 191584 | TCCCCAGGTGTCTCGAGA | 63 | 342 |
| 828578 | 1229 | 1246 | 191628 | 191645 | CTCTCGGTGCTTGGCCTC | 63 | 343 |
| 828583 | 1239 | 1256 | 191638 | 191655 | GGGACATTCTCTCTCGGT | 65 | 344 |
| 828585 | 1451 | 1468 | 198890 | 198907 | GGTGATGTAGTTCTCCAG | 58 | 345 |
| 828591 | 1462 | 1479 | 198901 | 198918 | GCCTGCAGAGCGGTGATG | 100 | 346 |
| 828597 | 1517 | 1534 | 218270 | 218287 | TGCGCGGACATACTTCTT | 67 | 347 |
| 828603 | 1529 | 1546 | 218282 | 218299 | GTCCTTCTGTTCTGCGCG | 128 | 348 |
| 828608 | 1557 | 1574 | 218310 | 218327 | GCTCGAAATGCTTTAGGG | 39 | 349 |
| 828614 | 1572 | 1589 | 218325 | 218342 | CCACCATGCGCACATGCT | 28 | 350 |
| 828620 | 1580 | 1597 | 218333 | 218350 | CTTGGGATCCACCATGCG | 69 | 351 |
| 828626 | 1594 | 1611 | 218347 | 218364 | ATCTGAGCGGCTTTCTTG | 74 | 352 |
| 828632 | 1603 | 1620 | 218356 | 218373 | TGGGACCGGATCTGAGCG | 77 | 353 |
| 828643 | 1640 | 1657 | 219330 | 219347 | CATGCGCTCATAAATCAC | 52 | 354 |
| 828649 | 1694 | 1711 | 219384 | 219401 | CTGAATCTCCTCGGCCAC | 90 | 355 |
| 828655 | 1775 | 1792 | 262092 | 262109 | GATCCTTGGTTCACTAAT | 85 | 356 |
| 828661 | 1804 | 1821 | 262121 | 262138 | GATGGCATGAGAGCATCG | 88 | 357 |
| 828667 | 1841 | 1858 | 262158 | 262175 | GGGAAGGAGCTCCACGGT | 91 | 358 |
| 828672 | 1853 | 1870 | 262170 | 262187 | CTCTCCATTCACGGGAAG | 73 | 359 |
| 828678 | 1891 | 1908 | 262208 | 262225 | CCAAAAGAATGCCACGGC | 10 | 360 |
| 828684 | 1967 | 1984 | 268947 | 268964 | CAGTCCTCGGTCGGCAGC | 233 | 361 |
| 828690 | 1977 | 1994 | 268957 | 268974 | GTCGAGTGGTCAGTCCTC | 74 | 362 |
| 828695 | 1986 | 2003 | N/A | N/A | CAGAACCTGGTCGAGTGG | 90 | 363 |
| 828701 | 2013 | 2030 | 276343 | 276360 | TCTCCTCCGTCTTGATAT | 242 | 364 |
| 828707 | 2047 | 2064 | 276377 | 276394 | TGTCGGAATTCTGCATCC | 84 | 365 |
| 828713 | 2061 | 2078 | 276391 | 276408 | CATATCCTGAGTCATGTC | 67 | 366 |
| 828719 | 2121 | 2138 | 282155 | 282172 | TTGCACCTTTGTTTGAAC | 76 | 367 |
| 828725 | 2136 | 2153 | 282170 | 282187 | CCATGAGTCCAATGATTG | 85 | 368 |
| 828731 | 2148 | 2165 | 282182 | 282199 | CAACACCGCCCACCATGA | 257 | 369 |
| 828737 | 2166 | 2183 | 282200 | 282217 | CGATCACTGTCGCTATGA | 86 | 370 |
| 828743 | 2283 | 2300 | 292267 | 292284 | CGTTCTGCTGCATCTTGG | 90 | 371 |
| 828749 | 2310 | 2327 | 292294 | 292311 | AGAACTTGTAGGTTGGAT | 50 | 372 |
| 828755 | 892 | 909 | N/A | N/A | GAGCACACCTCTCGAACC | 85 | 373 |
| 828761 | 906 | 923 | 173825 | 173842 | TCTCGGCTTGTTCAGAGC | 62 | 374 |
| 828767 | 917 | 934 | 173836 | 173853 | GCACGGCCCCGTCTCGGC | 75 | 375 |
| 828773 | 932 | 949 | 173851 | 173868 | GGAGATCATTGCTCGGCA | 35 | 376 |
| 828779 | 942 | 959 | 173861 | 173878 | AGTACCAGCGGGAGATCA | 84 | 377 |
| 828784 | 969 | 986 | 173888 | 173905 | GGGCACACTTCCCTTCAG | 84 | 378 |
| 828788 | 996 | 1013 | 173915 | 173932 | TGCCGCCACATCCGCCGT | 64 | 379 |
| 828794 | 1036 | 1053 | 173955 | 173972 | ACGGCCATGCAGTACTCT | 53 | 380 |
| 828800 | 1048 | 1065 | 173967 | 173984 | GCGCTGCCACACACGGCC | 67 | 381 |
| 828806 | 1084 | 1101 | 176601 | 176618 | GGTTCCTGGGTAGTCTTG | 73 | 382 |
| 828812 | 1099 | 1116 | 176616 | 176633 | GGATCTCGGGCAAGAGGT | 80 | 383 |
| 828818 | 1124 | 1141 | N/A | N/A | GGCTGCTGTTGTAGGAAG | 73 | 384 |
| 828830 | N/A | N/A | 83927 | 83944 | GCAGTTCAGGGTAGACCT | 74 | 385 |

TABLE 7
Reduction of APP RNA by 5-8-5 gapmers with mixed wings and a mixed backbone

| | SEQ ID | SEQ ID | SEQ ID | SEQ ID | | APP | |
|---|---|---|---|---|---|---|---|
| | NO: 1 | NO: 1 | NO: 2 | NO: 2 | | RNA | SEQ |
| Compound | Start | Stop | Start | Stop | | (% | ID |
| ID | Site | Site | Site | Site | Sequence (5′ to 3′) | control) | NO: |
| 699500 | 307 | 324 | 83976 | 83993 | CAGTTCTGGATGGTCACT | 59 | 386 |
| 699509 | 317 | 334 | 83986 | 84003 | CCGCTTGCACCAGTTCTG | 43 | 387 |
| 699533 | 487 | 504 | N/A | N/A | GTCTCTTTGGCGACGGTG | 32 | 388 |
| 699572 | 985 | 1002 | 173904 | 173921 | CCGCCGTAAAAGAATGGG | 85 | 389 |
| 699592 | 1244 | 1261 | N/A | N/A | GACCTGGGACATTCTCTC | 58 | 390 |
| 699600 | 1257 | 1274 | 197971 | 197988 | CCCATTCTCTCATGACCT | 47 | 18 |
| 828406 | 44 | 61 | 3390 | 3407 | CAGGAGCAGTGCCAAACC | 45 | 391 |
| 828412 | 69 | 86 | 3415 | 3432 | GCGCCCGAGCCGTCCAGG | 51 | 392 |
| 828418 | 97 | 114 | 61860 | 61877 | CCAGCATTACCATCAGTG | 31 | 393 |
| 828424 | 200 | 217 | 61963 | 61980 | GGTTTTGGTCCCTGATGG | 55 | 394 |
| 828429 | 221 | 238 | 61984 | 62001 | GCCTTCCTTGGTATCAAT | 60 | 395 |
| 828435 | 238 | 255 | 62001 | 62018 | TGGCAATACTGCAGGATG | 27 | 396 |
| 828441 | 259 | 276 | N/A | N/A | TGCAGTTCAGGGTAGACT | 52 | 397 |
| 828447 | 285 | 302 | 83954 | 83971 | GGTTGGCTTCTACCACAT | 51 | 398 |
| 828453 | 295 | 312 | 83964 | 83981 | GTCACTGGTTGGTTGGCT | 78 | 399 |
| 828458 | 330 | 347 | 83999 | 84016 | ACTGCTTGCGGCCCCGCT | 66 | 400 |
| 828463 | 339 | 356 | 84008 | 84025 | GGGTCTTGCACTGCTTGC | 41 | 401 |
| 828469 | 368 | 385 | 84037 | 84054 | GCAGCGGTAGGGAATCAC | 85 | 402 |
| 828475 | 378 | 395 | N/A | N/A | CACCAACTAAGCAGCGGT | 102 | 403 |
| 828481 | 417 | 434 | 120680 | 120697 | TGCACTTGTCAGGAACGA | 43 | 404 |
| 828487 | 441 | 458 | 120704 | 120721 | CCATCCTCTCCTGGTGTA | 54 | 405 |
| 828493 | 464 | 481 | 120727 | 120744 | GTGAAGATGAGTTTCGCA | 88 | 406 |
| 828501 | 513 | 530 | 122818 | 122835 | GCAAGTTGGTACTCTTCT | 40 | 407 |
| 828507 | 530 | 547 | 122835 | 122852 | CAACATGCCGTAGTCATG | 44 | 408 |
| 828513 | 547 | 564 | 122852 | 122869 | TCAATTCCGCAGGGCAGC | 72 | 409 |
| 828519 | 562 | 579 | 122867 | 122884 | ACCCCTCGGAACTTGTCA | 66 | 410 |
| 828525 | 571 | 588 | 122876 | 122893 | ACAAACTCTACCCCTCGG | 90 | 411 |
| 828531 | 636 | 653 | 122941 | 122958 | CCGAGTCATCCTCCTCCG | 42 | 412 |
| 828537 | 652 | 669 | 122957 | 122974 | CCGCCCCACCAGACATCC | 44 | 413 |
| 828543 | 671 | 688 | 122976 | 122993 | TGCATAGTCTGTGTCTGC | 45 | 414 |
| 828549 | 682 | 699 | N/A | N/A | TCACTCCCATCTGCATAG | 94 | 415 |
| 828561 | 1128 | 1145 | 191527 | 191544 | TACTGGCTGCTGTTGTAG | 116 | 416 |
| 828567 | 1157 | 1174 | 191556 | 191573 | CTCGAGATACTTGTCAAC | 60 | 417 |
| 828573 | 1169 | 1186 | 191568 | 191585 | ATCCCCAGGTGTCTCGAG | 78 | 418 |
| 828579 | 1231 | 1248 | 191630 | 191647 | CTCTCTCGGTGCTTGGCC | 58 | 419 |
| 828586 | 1452 | 1469 | 198891 | 198908 | CGGTGATGTAGTTCTCCA | 71 | 420 |
| 828592 | 1474 | 1491 | 198913 | 198930 | CGAGGAGGAACAGCCTGC | 69 | 421 |
| 828598 | 1518 | 1535 | 218271 | 218288 | CTGCGCGGACATACTTCT | 61 | 422 |
| 828604 | 1531 | 1548 | 218284 | 218301 | CTGTCCTTCTGTTCTGCG | 57 | 423 |
| 828609 | 1559 | 1576 | 218312 | 218329 | ATGCTCGAAATGCTTTAG | 59 | 424 |
| 828615 | 1574 | 1591 | 218327 | 218344 | ATCCACCATGCGCACATG | 53 | 425 |
| 828621 | 1582 | 1599 | 218335 | 218352 | TTCTTGGGATCCACCATG | 112 | 426 |
| 828627 | 1595 | 1612 | 218348 | 218365 | GATCTGAGCGGCTTTCTT | 127 | 427 |
| 828633 | 1604 | 1621 | 218357 | 218374 | CTGGGACCGGATCTGAGC | 68 | 428 |
| 828638 | 1624 | 1641 | 219314 | 219331 | ACACGGAGGTGTGTCATA | 54 | 429 |
| 828644 | 1642 | 1659 | 219332 | 219349 | TTCATGCGCTCATAAATC | 100 | 430 |
| 828650 | 1696 | 1713 | 219386 | 219403 | TCCTGAATCTCCTCGGCC | 59 | 431 |
| 828656 | 1778 | 1795 | 262095 | 262112 | ACTGATCCTTGGTTCACT | 61 | 432 |
| 828662 | 1812 | 1829 | 262129 | 262146 | CGGTCAAAGATGGCATGA | 79 | 433 |
| 828668 | 1842 | 1859 | 262159 | 262176 | CGGGAAGGAGCTCCACGG | 100 | 434 |
| 828673 | 1856 | 1873 | 262173 | 262190 | GAACTCTCCATTCACGGG | 113 | 435 |
| 828679 | 1939 | 1956 | N/A | N/A | TCAACAGGCTCAACTTCG | 178 | 436 |
| 828685 | 1969 | 1986 | 268949 | 268966 | GTCAGTCCTCGGTCGGCA | 138 | 437 |
| 828691 | 1978 | 1995 | 268958 | 268975 | GGTCGAGTGGTCAGTCCT | 158 | 438 |
| 828696 | 1987 | 2004 | N/A | N/A | CCAGAACCTGGTCGAGTG | 124 | 439 |
| 828702 | 2014 | 2031 | 276344 | 276361 | ATCTCCTCCGTCTTGATA | 322 | 440 |
| 828708 | 2051 | 2068 | 276381 | 276398 | GTCATGTCGGAATTCTGC | 49 | 441 |
| 828714 | 2067 | 2084 | 276397 | 276414 | GAACTTCATATCCTGAGT | 99 | 442 |
| 828720 | 2123 | 2140 | 282157 | 282174 | GATTGCACCTTTGTTTGA | 306 | 443 |
| 828726 | 2138 | 2155 | 282172 | 282189 | CACCATGAGTCCAATGAT | 114 | 444 |
| 828732 | 2151 | 2168 | 282185 | 282202 | TGACAACACCGCCCACCA | 102 | 445 |
| 828738 | 2170 | 2187 | 282204 | 282221 | ATGACGATCACTGTCGCT | 96 | 446 |
| 828744 | 2285 | 2302 | 292269 | 292286 | GCCGTTCTGCTGCATCTT | 152 | 447 |
| 828750 | 883 | 900 | N/A | N/A | TCTCGAACCACCTCTTCC | 100 | 448 |
| 828756 | 895 | 912 | N/A | N/A | TCAGAGCACACCTCTCGA | 109 | 449 |
| 828762 | 909 | 926 | 173828 | 173845 | CCGTCTCGGCTTGTTCAG | 64 | 450 |
| 828768 | 918 | 935 | 173837 | 173854 | GGCACGGCCCCGTCTCGG | 70 | 451 |
| 828774 | 933 | 950 | 173852 | 173869 | GGGAGATCATTGCTCGGC | 56 | 452 |
| 828780 | 945 | 962 | 173864 | 173881 | CAAAGTACCAGCGGGAGA | 79 | 453 |
| 828789 | 999 | 1016 | 173918 | 173935 | GGTTGCCGCCACATCCGC | 98 | 454 |
| 828795 | 1037 | 1054 | 173956 | 173973 | CACGGCCATGCAGTACTC | 58 | 455 |
| 828801 | 1062 | 1079 | N/A | N/A | AACTTTGGGACATGGCGC | 89 | 456 |
| 828807 | 1086 | 1103 | 176603 | 176620 | GAGGTTCCTGGGTAGTCT | 86 | 457 |
| 828813 | 1101 | 1118 | 176618 | 176635 | CAGGATCTCGGGCAAGAG | 68 | 458 |
| 828819 | N/A | N/A | 33809 | 33826 | AAGTCCTCTAATTGGTCC | 82 | 459 |

TABLE 8
Reduction of APP RNA by 5-8-5 gapmers with mixed wings and a mixed backbone

| | SEQ ID | SEQ ID | SEQ ID | SEQ ID | | APP | |
|---|---|---|---|---|---|---|---|
| | NO: 3 | NO: 3 | NO: 4 | NO: 4 | | RNA | SEQ |
| Compound | Start | Stop | Start | Stop | | (% | ID |
| ID | Site | Site | Site | Site | Sequence (5′ to 3′) | control) | NO |
| 828554 | N/A | N/A | 999 | 1016 | GGAACTCGAACCACCTCT | 121 | 460 |
| 828555 | N/A | N/A | 1001 | 1018 | TAGGAACTCGAACCACCT | 87 | 461 |
| 828556 | N/A | N/A | 1002 | 1019 | GTAGGAACTCGAACCACC | 56 | 462 |
| 828557 | N/A | N/A | 1005 | 1022 | GTTGTAGGAACTCGAACC | 88 | 463 |
| 828558 | N/A | N/A | 1008 | 1025 | GCTGTTGTAGGAACTCGA | 79 | 464 |
| 828559 | N/A | N/A | 1010 | 1027 | CTGCTGTTGTAGGAACTC | 43 | 465 |
| 828824 | 1195 | 1212 | N/A | N/A | CTGTTGTAGGAATGGCGC | 145 | 468 |
| 828825 | 1200 | 1217 | N/A | N/A | GGCTGCTGTTGTAGGAAT | 57 | 469 |

TABLE 9
Reduction of APP RNA by 5-8-5 gapmers with mixed wings and a mixed backbone

| | SEQ ID | SEQ ID | SEQ ID | SEQ ID | SEQ ID | SEQ ID | | APP | |
|---|---|---|---|---|---|---|---|---|---|
| | NO: 5 | NO: 5 | NO: 6 | NO: 6 | NO: 7 | NO: 7 | | RNA | SEQ |
| Compound | Start | Stop | Start | Stop | Start | Stop | | (% | ID |
| ID | Site | Site | Site | Site | Site | Site | Sequence (5′ to 3′) | control) | NO |
| 828822 | 241 | 258 | N/A | N/A | N/A | N/A | CAGTGGGTACATAGTTGA | 122 | 466 |
| 828823 | 243 | 260 | N/A | N/A | N/A | N/A | CCATCAGTGGGTACATAG | 57 | 467 |
| 828826 | N/A | N/A | 176 | 193 | N/A | N/A | AGGGTAGACCTCCAGCGC | 57 | 470 |
| 828827 | N/A | N/A | 178 | 195 | N/A | N/A | TCAGGGTAGACCTCCAGC | 80 | 471 |
| 828828 | N/A | N/A | 180 | 197 | N/A | N/A | GTTCAGGGTAGACCTCCA | 84 | 472 |
| 828829 | N/A | N/A | 182 | 199 | N/A | N/A | CAGTTCAGGGTAGACCTC | 67 | 473 |
| 828831 | N/A | N/A | N/A | N/A | 1952 | 1969 | GTCAACCCAGAACCTTCG | 121 | 474 |

### Example 2: Effect of Modified Oligonucleotides on Human APP In Vitro, Multiple Doses

Modified oligonucleotides selected from the examples above were tested at various doses in SH-S5Y cells. Cells were plated at a density of 20,000 cells per well and treated by electroporation with various modified oligonucleotides, as specified in the tables below. After a treatment period of approximately 24 hours, total RNA was isolated from the cells and APP RNA levels were measured by quantitative real-time PCR. Human APP primer probe set HTS96, described herein above, was used to measure RNA levels. APP RNA levels were normalized to GADPH. Results are presented in the tables below as percent APP RNA levels relative to untreated control cells. The half maximal inhibitory concentration (IC₅₀) of each modified oligonucleotide is also presented. IC₅₀ was calculated using a linear regression on a log/linear plot of the data in excel. ‘N.D.’ (‘no data’) indicates that the % inhibition was not determined for that particular modified oligonucleotide in that particular experiment. ‘N.C.’ (“no calculation”) indicates that the range of concentrations tested was not sufficient for an accurate calculation of IC₅₀.

TABLE 10
Dose-dependent reduction of human APP RNA expression
in SH-S5Y cells

| | APP RNA Expression (% control) | | | | |
|---|---|---|---|---|---|
| Compound ID | 0.31 nM | 1.25 nM | 5.0 nM | 20.0 nM | IC50(μM) |
| 828425 | 87 | 55 | 36 | 18 | 2.3 |
| 828426 | 84 | 87 | 54 | 27 | 6.6 |
| 828443 | 126 | 107 | 144 | 62 | N.C. |
| 828444 | 108 | 83 | 51 | 26 | 5.8 |
| 828455 | 83 | 96 | 58 | 25 | 7.3 |
| 828464 | 71 | 43 | 18 | 7 | 0.9 |
| 828490 | 61 | 38 | 34 | 19 | 0.7 |
| 828527 | 85 | 52 | 21 | 30 | 1.4 |
| 828528 | 97 | 63 | 37 | 20 | 3.2 |
| 828532 | 74 | 49 | 44 | 11 | 1.4 |
| 828533 | 64 | 49 | 40 | 10 | 1.2 |
| 828545 | 78 | 52 | 27 | 14 | 1.5 |
| 828546 | 110 | 62 | 72 | 52 | N.C. |
| 828550 | 103 | 75 | 82 | 59 | N.C. |
| 828574 | 59 | 38 | 22 | 21 | 0.6 |
| 828606 | 151 | 107 | 76 | 63 | N.C. |
| 828610 | 129 | 81 | 58 | 34 | 7.8 |
| 828645 | 69 | 58 | 25 | 20 | 1.5 |

TABLE 11
Dose-dependent reduction of human APP RNA expression
in SH-S5Y cells

| | APP RNA Expression (% control) | | | | |
|---|---|---|---|---|---|
| | 0.31 nM | 1.25 nM | 5.0 nM | 20.0 nM | IC50(μM) |
| 699533 | 63 | 33 | 25 | 13 | 0.6 |
| 828404 | 78 | 56 | 30 | N.D. | 1.7 |
| 828417 | 92 | 57 | 48 | 19 | 3.2 |
| 828418 | 63 | 40 | 27 | 10 | 0.7 |
| 828422 | 68 | 36 | 23 | 8 | 0.8 |
| 828428 | 44 | 29 | 40 | 13 | 0.1 |
| 828435 | 83 | 55 | 34 | 34 | 1.9 |
| 828445 | 113 | 52 | 37 | 6 | 2.7 |
| 828463 | 103 | 103 | 13 | N.D. | 2.6 |
| 828480 | 83 | 56 | 30 | 11 | 1.9 |
| 828499 | 86 | 66 | 32 | 11 | 2.3 |
| 828501 | 134 | 47 | 38 | 9 | 3.2 |
| 828531 | 77 | 76 | 39 | 54 | 3.6 |
| 828541 | 70 | 69 | 40 | 31 | 3.1 |
| 828542 | 60 | 51 | 31 | 7 | 0.9 |
| 828565 | 46 | 27 | 14 | 5 | 0.2 |
| 828614 | 63 | 40 | 26 | 9 | 0.7 |
| 828645 | 94 | 73 | 35 | 27 | 3.8 |
| 828773 | 61 | 39 | 43 | 17 | 0.8 |

### Example 3: Effect of Mixed Wing and Mixed Backbone or MOE and Mixed Backbone Modified Oligonucleotides on Human APP RNA In Vitro, Single Dose

Modified oligonucleotides complementary to human APP were synthesized with chemical modification patterns as indicated in the table below. The modified oligonucleotides in the table below are gapmers. The gapmers have a central gap segment that comprises 2′-deoxynucleosides and is flanked by wing segments on both the 5′ end on the 3′ end comprising and cEt nucleosides and/or 2′-MOE nucleosides. All cytosine residues throughout each gapmer are 5′-methyl cytosines. The internucleoside linkages are mixed phosphodiester internucleoside linkages and phosphorothioate internucleoside linkages.

Cultured SH-SY5Y cells at a density of 20,000 cells per well were treated with 4,000 nM of modified oligonucleotide by electroporation. After a treatment period of approximately 24 hours, total RNA was isolated from the cells and APP RNA levels were measured by quantitative real-time RTPCR. Human APP primer probe set RTS35571 (forward sequence CCCACTTTGTGATTCCCTACC, designated herein as SEQ ID NO: 913; reverse sequence ATCCATCCTCTCCTGGTGTAA, designated herein as SEQ ID NO: 914; probe sequence TGATGCCCTTCTCGTTCCTGACAA, designated herein as SEQ ID NO: 915) was used to measure RNA levels. APP RNA levels were adjusted according to total RNA content, as measured by RIBOGREEN®. Results are presented in the tables below as percent APP RNA levels relative to untreated control cells.

Modified oligonucleotides in Table 12 below are 18 nucleosides in length and have the sugar motif eeeeeddddddddkeeee, wherein each “e” is nucleoside comprising a 2′-MOE sugar moiety, each “k” is a nucleoside comprising a cEt sugar moiety, and each “d” is a nucleoside comprising a 2′-β-D-deoxyribosyl sugar moiety. The internucleoside linkage motif is sososssssssssosss, wherein each “s” represents a phosphorothioate internucleoside linkage and each “o” represents a phosphodiester internucleoside linkage. All cytosine residues are 5-methylcytosines. “Start Site” indicates the 5′-most nucleoside to which the gapmer is complementary in the human nucleic acid sequence. “Stop Site” indicates the 3′-most nucleoside to which the gapmer is complementary in the human nucleic acid sequence.

TABLE 12
Reduction of APP with 5-8-5 gapmers with mixed wings and a mixed backbone

| | SEQ ID | SEQ ID | SEQ ID | SEQ ID | | APP | |
|---|---|---|---|---|---|---|---|
| | NO: 1 | NO: 1 | NO: 2 | NO: 2 | | RNA | SEQ |
| Compound | Start | Stop | Start | Stop | | (% | ID |
| ID | Site | Site | Site | Site | Sequence (5′ to 3′) | control) | NO: |
| 912249 | 487 | 504 | N/A | N/A | GTCTCTTTGGCGACGGTG | 99 | 388 |
| 912250 | 488 | 505 | N/A | N/A | TGTCTCTTTGGCGACGGT | 36 | 475 |
| 912251 | 489 | 506 | N/A | N/A | ATGTCTCTTTGGCGACGG | 29 | 16 |
| 912252 | 490 | 507 | N/A | N/A | CATGTCTCTTTGGCGACG | 36 | 476 |
| 912253 | 491 | 508 | N/A | N/A | GCATGTCTCTTTGGCGAC | 33 | 90 |
| 912254 | 492 | 509 | N/A | N/A | TGCATGTCTCTTTGGCGA | 27 | 477 |
| 912255 | 493 | 510 | N/A | N/A | CTGCATGTCTCTTTGGCG | 19 | 165 |
| 912256 | 657 | 674 | 122962 | 122979 | CTGCTCCGCCCCACCAGA | 52 | 116 |
| 912257 | 659 | 676 | 122964 | 122981 | GTCTGCTCCGCCCCACCA | 64 | 478 |
| 912258 | 661 | 678 | 122966 | 122983 | GTGTCTGCTCCGCCCCAC | 32 | 479 |
| 912259 | 662 | 679 | 122967 | 122984 | TGTGTCTGCTCCGCCCCA | 30 | 191 |
| 912260 | 663 | 680 | 122968 | 122985 | CTGTGTCTGCTCCGCCCC | 46 | 480 |
| 912261 | 664 | 681 | 122969 | 122986 | TCTGTGTCTGCTCCGCCC | 99 | 265 |
| 912262 | 665 | 682 | 122970 | 122987 | GTCTGTGTCTGCTCCGCC | 17 | 481 |
| 912263 | 666 | 683 | 122971 | 122988 | AGTCTGTGTCTGCTCCGC | 15 | 338 |
| 912264 | 667 | 684 | 122972 | 122989 | TAGTCTGTGTCTGCTCCG | 43 | 482 |
| 912265 | 668 | 685 | 122973 | 122990 | ATAGTCTGTGTCTGCTCC | 43 | 483 |
| 912266 | 669 | 686 | 122974 | 122991 | CATAGTCTGTGTCTGCTC | 28 | 484 |
| 912267 | 670 | 687 | 122975 | 122992 | GCATAGTCTGTGTCTGCT | 24 | 485 |
| 912268 | 671 | 688 | 122976 | 122993 | TGCATAGTCTGTGTCTGC | 33 | 414 |
| 912269 | 672 | 689 | 122977 | 122994 | CTGCATAGTCTGTGTCTG | 41 | 486 |
| 912270 | 674 | 691 | 122979 | 122996 | ATCTGCATAGTCTGTGTC | 60 | 43 |
| 912271 | 676 | 693 | 122981 | 122998 | CCATCTGCATAGTCTGTG | 24 | 117 |
| 912272 | 678 | 695 | 122983 | 123000 | TCCCATCTGCATAGTCTG | 17 | 487 |
| 912273 | 1614 | 1631 | N/A | N/A | GTGTCATAACCTGGGACC | 36 | 488 |
| 912274 | 1616 | 1633 | N/A | N/A | GTGTGTCATAACCTGGGA | 41 | 489 |
| 912275 | 1618 | 1635 | N/A | N/A | AGGTGTGTCATAACCTGG | 61 | 490 |
| 912276 | 1620 | 1637 | 219310 | 219327 | GGAGGTGTGTCATAACCT | 52 | 491 |
| 912277 | 1622 | 1639 | 219312 | 219329 | ACGGAGGTGTGTCATAAC | 56 | 492 |
| 912278 | 1624 | 1641 | 219314 | 219331 | ACACGGAGGTGTGTCATA | 52 | 429 |
| 912279 | 1626 | 1643 | 219316 | 219333 | TCACACGGAGGTGTGTCA | 69 | 493 |
| 912280 | 1628 | 1645 | 219318 | 219335 | AATCACACGGAGGTGTGT | 51 | 494 |
| 912281 | 1630 | 1647 | 219320 | 219337 | TAAATCACACGGAGGTGT | 45 | 495 |
| 912282 | 1631 | 1648 | 219321 | 219338 | ATAAATCACACGGAGGTG | 68 | 496 |
| 912283 | 1632 | 1649 | 219322 | 219339 | CATAAATCACACGGAGGT | 352 | 132 |
| 912284 | 1633 | 1650 | 219323 | 219340 | TCATAAATCACACGGAGG | 362 | 497 |
| 912285 | 1634 | 1651 | 219324 | 219341 | CTCATAAATCACACGGAG | 32 | 498 |
| 912286 | 1635 | 1652 | 219325 | 219342 | GCTCATAAATCACACGGA | 26 | 207 |
| 912287 | 1636 | 1653 | 219326 | 219343 | CGCTCATAAATCACACGG | 85 | 499 |
| 912288 | 1637 | 1654 | 219327 | 219344 | GCGCTCATAAATCACACG | 150 | 281 |
| 912289 | 1638 | 1655 | 219328 | 219345 | TGCGCTCATAAATCACAC | 63 | 500 |
| 912290 | 1639 | 1656 | 219329 | 219346 | ATGCGCTCATAAATCACA | 56 | 501 |
| 912291 | 1640 | 1657 | 219330 | 219347 | CATGCGCTCATAAATCAC | 61 | 354 |

Modified oligonucleotides in Table 13 below are 20 nucleosides in length and are 5-10-5 MOE gapmers. The internucleoside linkage motif is sososssssssssssosss, wherein each “s” represents a phosphorothioate internucleoside linkage and each “o” represents a phosphodiester internucleoside linkage. All cytosine residues are 5-methylcytosines. “Start Site” indicates the 5′-most nucleoside to which the gapmer is complementary in the human nucleic acid sequence. “Stop Site” indicates the 3′-most nucleoside to which the gapmer is complementary in the human nucleic acid sequence.

TABLE 13
Reduction of APP RNA by 5-10-5 MOE gapmers having a mixed backbone

| | SEQ ID | SEQ ID | SEQ ID | SEQ ID | | APP | |
|---|---|---|---|---|---|---|---|
| | NO: 1 | NO: 1 | NO: 2 | NO: 2 | | RNA | SEQ |
| Compound | Start | Stop | Start | Stop | | (% | ID |
| ID | Site | Site | Site | Site | Sequence (5′ to 3′) | control) | NO: |
| 912292 | 486 | 505 | N/A | N/A | TGTCTCTTTGGCGACGGTGT | 37 | 502 |
| 912293 | 490 | 509 | N/A | N/A | TGCATGTCTCTTTGGCGACG | 25 | 503 |
| 912294 | 492 | 511 | N/A | N/A | ACTGCATGTCTCTTTGGCGA | 23 | 504 |
| 912295 | 663 | 682 | 122968 | 122985 | GTCTGTGTCTGCTCCGCCCC | 15 | 505 |
| 912296 | 665 | 684 | 122970 | 122987 | TAGTCTGTGTCTGCTCCGCC | 32 | 506 |
| 912297 | 670 | 689 | 122975 | 122992 | CTGCATAGTCTGTGTCTGCT | 40 | 507 |
| 912298 | 1634 | 1653 | 219324 | 219341 | CGCTCATAAATCACACGGAG | 18 | 508 |
| 912299 | 1635 | 1654 | 219325 | 219342 | GCGCTCATAAATCACACGGA | 30 | 509 |
| 912300 | 1636 | 1655 | 219326 | 219343 | TGCGCTCATAAATCACACGG | 67 | 510 |
| 912301 | 1633 | 1652 | 219323 | 219340 | GCTCATAAATCACACGGAGG | 23 | 511 |
| 912302 | 1632 | 1651 | 219322 | 219339 | CTCATAAATCACACGGAGGT | 72 | 512 |
| 912303 | 669 | 688 | 122974 | 122991 | TGCATAGTCTGTGTCTGCTC | 27 | 513 |
| 912304 | 668 | 687 | 122973 | 122990 | GCATAGTCTGTGTCTGCTCC | 28 | 514 |
| 912305 | 671 | 690 | 122976 | 122993 | TCTGCATAGTCTGTGTCTGC | 42 | 515 |
| 912306 | 672 | 691 | 122977 | 122994 | ATCTGCATAGTCTGTGTCTG | 48 | 516 |

### Example 4: Design of RNAi Compounds with Antisense RNAi Oligonucleotides Complementary to a Human APP Nucleic Acid

RNAi compounds comprising antisense RNAi oligonucleotides complementary to a human APP nucleic acid and sense RNAi oligonucleotides complementary to the antisense RNAi oligonucleotides were designed as follows.

The RNAi compounds in the tables below consist of an antisense RNAi oligonucleotide and a sense RNAi oligonucleotide, wherein, in each case the antisense RNAi oligonucleotides is 23 nucleosides in length; has a sugar motif (from 5′ to 3′) of: mfmfmfmfmfmfmfmfmfmfmmm; wherein “m” represents a 2′-O methylribosyl sugar, and the “f” represents a 2′-fluororibosyl sugar; and a linkage motif (from 5′ to 3′) of: ssooooooooooooooooooss; wherein ‘o’ represents a phosphodiester internucleoside linkage and ‘s’ represents a phosphorothioate internucleoside linkage. The sense RNAi oligonucleotides in each case is 21 nucleosides in length; has a sugar motif (from 5′ to 3′) of: fmfmfmfmfmfmfmfmfmfmf; wherein “m” represents a 2′-O methylribosyl sugar, and the “f” represents a 2′-fluororibosyl sugar; and a linkage motif (from 5′ to 3′) of: ssooooooooooooooooss; wherein ‘o’ represents a phosphodiester internucleoside linkage and ‘s’ represents a phosphorothioate internucleoside linkage. Each antisense RNAi oligonucleotides is complementary to the target nucleic acid (APP), and each sense RNAi oligonucleotides is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

“Start site” indicates the 5′-most nucleoside to which the antisense RNAi oligonucleotides is complementary in the human gene sequence. “Stop site” indicates the 3′-most nucleoside to which the antisense RNAi oligonucleotide is complementary in the human gene sequence. Each modified antisense RNAi oligonucleoside listed in the Tables below is 100% complementary to either SEQ ID NO: 1 (described herein above), SEQ ID NO: 2 (described herein above) or SEQ ID No:3 (described herein above) as indicated in the tables below.

TABLE 14
RNAi compounds targeting human APP SEQ ID No: 1

| | | | | SEQ ID | SEQ ID | | | |
|---|---|---|---|---|---|---|---|---|
| | | Antisense | SEQ | NO: 1 | NO: 1 | | | |
| Compound | Antisense | Sequence | ID | Antisense | Antisense | Sense | Sense Sequence | SEQ |
| Number | oligo ID | (5′ to 3′) | NO | Start Site | Stop Site | oligo ID | (5′ to 3′) | ID NO |
| 1381709 | 1381714 | AGCAGUGCCAAAC | 517 | 35 | 57 | 1381715 | GCUGCCCGGUUU | 666 |
| | | CGGGCAGCAU | | | | | GGCACUGCU | |
| 1381710 | 1381713 | AUCGCGACCCUGC | 518 | 14 | 36 | 1381711 | UGCCCCGCGCAG | 667 |
| | | GCGGGGCACC | | | | | GGUCGCGAU | |
| 1381712 | 1381717 | GCCGUCCAGGCGG | 519 | 56 | 78 | 1381716 | CCUGCUGGCCGC | 668 |
| | | CCAGCAGGAG | | | | | CUGGACGGC | |
| 1381718 | 1381720 | UUGUCAACGGCAU | 520 | 1142 | 1164 | 1381719 | UACCCCUGAUGC | 669 |
| | | CAGGGGUACU | | | | | CGUUGACAA | |
| 1381721 | 1381722 | AAAUGGGCAUGUU | 521 | 1184 | 1206 | 1381724 | UGAGAAUGAACA | 670 |
| | | CAUUCUCAUC | | | | | UGCCCAUUU | |
| 1381723 | 1381726 | UCCCCAGGUGUCU | 522 | 1163 | 1185 | 1381728 | GUAUCUCGAGAC | 671 |
| | | CGAGAUACUU | | | | | ACCUGGGGA | |
| 1381725 | 1381730 | AGCCUCUCUUUGG | 523 | 1205 | 1227 | 1381729 | CCAGAAAGCCAA | 672 |
| | | CUUUCUGGAA | | | | | AGAGAGGCU | |
| 1381727 | 1381732 | CUCUCUCGGUGCU | 524 | 1226 | 1248 | 1381731 | UGAGGCCAAGCA | 673 |
| | | UGGCCUCAAG | | | | | CCGAGAGAG | |
| 1381733 | 1381742 | AGCAGGCCAGCAU | 525 | 98 | 120 | 1381739 | UGAUGGUAAUGC | 674 |
| | | UACCAUCAGU | | | | | UGGCCUGCU | |
| 1381734 | 1381737 | GUGGGUACCUCCA | 526 | 77 | 99 | 1381738 | UCGGGCGCUGGA | 675 |
| | | GCGCCCGAGC | | | | | GGUACCCAC | |
| 1381735 | 1381743 | CCAUUCUGGACAU | 527 | 161 | 183 | 1381741 | GCACAUGAAUGU | 676 |
| | | UCAUGUGCAU | | | | | CCAGAAUGG | |
| 1381736 | 1381745 | AUGUUCAGUCUGC | 528 | 140 | 162 | 1381744 | GUUCUGUGGCAG | 677 |
| | | CACAGAACAU | | | | | ACUGAACAU | |
| 1381740 | 1381748 | AUGGCAAUCUGGG | 529 | 119 | 141 | 1381750 | GGCUGAACCCCA | 678 |
| | | GUUCAGCCAG | | | | | GAUUGCCAU | |
| 1381746 | 1381749 | UCUCUCAUGACCU | 530 | 1247 | 1269 | 1381747 | AAUGUCCCAGGU | 679 |
| | | GGGACAUUCU | | | | | CAUGAGAGA | |
| 1381751 | 1381754 | UGACGUUCUGCCU | 531 | 1268 | 1290 | 1381753 | AUGGGAAGAGGC | 680 |
| | | CUUCCCAUUC | | | | | AGAACGUCA | |
| 1381752 | 1381762 | GCUUUAGGCAAGU | 532 | 1289 | 1311 | 1381764 | AGCAAAGAACUU | 681 |
| | | UCUUUGCUUG | | | | | GCCUAAAGC | |
| 1381755 | 1381758 | GAUGGAUCUGAAU | 533 | 182 | 204 | 1381757 | GAAGUGGGAUUC | 682 |
| | | CCCACUUCCC | | | | | AGAUCCAUC | |
| 1381756 | 1381760 | UGGAUAACUGCCU | 534 | 1310 | 1332 | 1381763 | UGAUAAGAAGGC | 683 |
| | | UCUUAUCAGC | | | | | AGUUAUCCA | |
| 1381759 | 1381767 | UCCACUUUCUCCU | 535 | 1331 | 1353 | 1381768 | GCAUUUCCAGGA | 684 |
| | | GGAAAUGCUG | | | | | GAAAGUGGA | |
| 1381761 | 1381765 | GCUGCUUCCUGUU | 536 | 1352 | 1374 | 1381766 | AUCUUUGGAACA | 685 |
| | | CCAAAGAUUC | | | | | GGAAGCAGC | |
| 1381771 | 1381769 | UCAAUGCAGGUUU | 537 | 203 | 225 | 1381770 | AGGGACCAAAAC | 686 |
| | | UGGUCCCUGA | | | | | CUGCAUUGA | |
| 1381772 | 1381777 | GGGUAGACUUCUU | 538 | 245 | 267 | 1381775 | GUAUUGCCAAGA | 687 |
| | | GGCAAUACUG | | | | | AGUCUACCC | |
| 1381773 | 1381780 | UGCAGGAUGCCUU | 539 | 224 | 246 | 1381774 | UACCAAGGAAGG | 688 |
| | | CCUUGGUAUC | | | | | CAUCCUGCA | |
| 1381776 | 1381785 | ACAUUGGUGAUCU | 540 | 266 | 288 | 1381783 | UGAACUGCAGAU | 689 |
| | | GCAGUUCAGG | | | | | CACCAAUGU | |
| 1381778 | 1381779 | ACUGGUUGGUUGG | 541 | 287 | 309 | 1381781 | GGUAGAAGCCAA | 690 |
| | | CUUCUACCAC | | | | | CCAACCAGU | |
| 1381782 | 1381786 | ACCAGCUGCUGUC | 542 | 1373 | 1395 | 1381784 | CAACGAGAGACA | 691 |
| | | UCUCGUUGGC | | | | | GCAGCUGGU | |
| 1381789 | 1381787 | UUGCACCAGUUCU | 543 | 308 | 330 | 1381788 | GACCAUCCAGAA | 692 |
| | | GGAUGGUCAC | | | | | CUGGUGCAA | |
| 1381790 | 1381799 | UUGCACUGCUUGC | 544 | 329 | 351 | 1381800 | GCGGGGCCGCAA | 693 |
| | | GGCCCCGCUU | | | | | GCAGUGCAA | |
| 1381791 | 1381793 | CGGUCAUUGAGCA | 545 | 1415 | 1437 | 1381794 | GGAAGCCAUGCU | 694 |
| | | UGGCUUCCAC | | | | | CAAUGACCG | |
| 1381792 | 1381797 | ACUCUGGCCAUGU | 546 | 1394 | 1416 | 1381796 | GGAGACACACAU | 695 |
| | | GUGUCUCCAC | | | | | GGCCAGAGU | |
| 1381795 | 1381804 | UUCUCCAGGGCCA | 547 | 1436 | 1458 | 1381803 | CCGCCGCCUGGC | 696 |
| | | GGCGGCGGCG | | | | | CCUGGAGAA | |
| 1381798 | 1381801 | AUCACAAAGUGGG | 548 | 350 | 372 | 1381806 | GACCCAUCCCCA | 697 |
| | | GAUGGGUCUU | | | | | CUUUGUGAU | |
| 1381802 | 1381807 | GCCUGCAGAGCGG | 549 | 1457 | 1479 | 1381805 | CUACAUCACCGC | 698 |
| | | UGAUGUAGUU | | | | | UCUGCAGGC | |
| 1381808 | 1381815 | UGACGAGGCCGAG | 550 | 1478 | 1500 | 1381819 | UGUUCCUCCUCG | 699 |
| | | GAGGAACAGC | | | | | GCCUCGUCA | |
| 1381809 | 1381812 | AGGGCAUCACUUA | 551 | 392 | 414 | 1381811 | UGAGUUUGUAA | 700 |
| | | CAAACUCACC | | | | | GUGAUGCCCU | |
| 1381810 | 1381816 | CCAACUAAGCAGC | 552 | 371 | 393 | 1381813 | UCCCUACCGCUG | 701 |
| | | GGUAGGGAAU | | | | | CUUAGUUGG | |
| 1381817 | 1381820 | AUCCUCUCCUGGU | 553 | 434 | 456 | 1381814 | AUUCUUACACCA | 702 |
| | | GUAAGAAUUU | | | | | GGAGAGGAU | |
| 1381818 | 1381823 | UUGCACUUGUCAG | 554 | 413 | 435 | 1381821 | UCUCGUUCCUGA | 703 |
| | | GAACGAGAAG | | | | | CAAGUGCAA | |
| 1381825 | 1381824 | AGAUGAGUUUCGC | 555 | 455 | 477 | 1381822 | GGAUGUUUGCGA | 704 |
| | | AAACAUCCAU | | | | | AACUCAUCU | |
| 1381826 | 1381829 | UUCUUUAGCAUAU | 556 | 1499 | 1521 | 1381827 | CGUGUUCAAUAU | 705 |
| | | UGAACACGUG | | | | | GCUAAAGAA | |
| 1381828 | 1381833 | UUCUGUUCUGCGC | 557 | 1520 | 1542 | 1381834 | GUAUGUCCGCGC | 706 |
| | | GGACAUACUU | | | | | AGAACAGAA | |
| 1381830 | 1381836 | UUGGCGACGGUGU | 558 | 476 | 498 | 1381831 | UCACUGGCACAC | 707 |
| | | GCCAGUGAAG | | | | | CGUCGCCAA | |
| 1381832 | 1381837 | CUCUUCUCACUGC | 559 | 497 | 519 | 1381838 | AGAGACAUGCAG | 708 |
| | | AUGUCUCUUU | | | | | UGAGAAGAG | |
| 1381835 | 1381843 | UUUAGGGUGUGCU | 560 | 1541 | 1563 | 1381839 | GGACAGACAGCA | 709 |
| | | GUCUGUCCUU | | | | | CACCCUAAA | |
| 1381840 | 1381841 | AUGCGCACAUGCU | 561 | 1562 | 1584 | 1381845 | GCAUUUCGAGCA | 710 |
| | | CGAAAUGCUU | | | | | UGUGCGCAU | |
| 1381842 | 1381846 | GCGGCUUUCUUGG | 562 | 1583 | 1605 | 1381847 | GGUGGAUCCCAA | 711 |
| | | GAUCCACCAU | | | | | GAAAGCCGC | |
| 1381844 | 1381849 | AUAACCUGGGACC | 563 | 1604 | 1626 | 1381848 | UCAGAUCCGGUC | 712 |
| | | GGAUCUGAGC | | | | | CCAGGUUAU | |
| 1381850 | 1381852 | UAAAUCACACGGA | 564 | 1625 | 1647 | 1381851 | GACACACCUCCG | 713 |
| | | GGUGUGUCAU | | | | | UGUGAUUUA | |
| 1381853 | 1381856 | AGAGACUGAUUCA | 565 | 1646 | 1668 | 1381857 | UGAGCGCAUGAA | 714 |
| | | UGCGCUCAUA | | | | | UCAGUCUCU | |
| 1381854 | 1381859 | GGCACGUUGUAGA | 566 | 1667 | 1689 | 1381860 | CUCCCUGCUCUA | 715 |
| | | GCAGGGAGAG | | | | | CAACGUGCC | |
| 1381855 | 1381866 | UGAAUCUCCUCGG | 567 | 1688 | 1710 | 1381867 | UGCAGUGGCCGA | 716 |
| | | CCACUGCAGG | | | | | GGAGAUUCA | |
| 1381858 | 1381865 | AGCAGCUCAUCAA | 568 | 1709 | 1731 | 1381862 | GGAUGAAGUUG | 717 |
| | | CUUCAUCCUG | | | | | AUGAGCUGCU | |
| 1381861 | 1381869 | GAAUAGUUUUGCU | 569 | 1730 | 1752 | 1381863 | UCAGAAAGAGCA | 718 |
| | | CUUUCUGAAG | | | | | AAACUAUUC | |
| 1381864 | 1381870 | AUGUUGGCCAAGA | 570 | 1751 | 1773 | 1381868 | AGAUGACGUCUU | 719 |
| | | CGUCAUCUGA | | | | | GGCCAACAU | |
| 1381872 | 1381871 | CUGAUCCUUGGUU | 571 | 1772 | 1794 | 1381873 | GAUUAGUGAACC | 720 |
| | | CACUAAUCAU | | | | | AAGGAUCAG | |
| 1381874 | 1381878 | AUGAGAGCAUCGU | 572 | 1793 | 1815 | 1381877 | UUACGGAAACGA | 721 |
| | | UUCCGUAACU | | | | | UGCUCUCAU | |
| 1381875 | 1381881 | GGAAGGAGCUCCA | 573 | 1835 | 1857 | 1381880 | AACCACCGUGGA | 722 |
| | | CGGUGGUUUU | | | | | GCUCCUUCC | |
| 1381876 | 1381883 | UUCGUUUCGGUCA | 574 | 1814 | 1836 | 1381885 | GCCAUCUUUGAC | 723 |
| | | AAGAUGGCAU | | | | | CGAAACGAA | |
| 1381879 | 1381884 | UGCCACGGCUGGA | 575 | 1877 | 1899 | 1381886 | GGACGAUCUCCA | 724 |
| | | GAUCGUCCAG | | | | | GCCGUGGCA | |
| 1381882 | 1381890 | AGGCUGAACUCUC | 576 | 1856 | 1878 | 1381891 | CGUGAAUGGAGA | 725 |
| | | CAUUCACGGG | | | | | GUUCAGCCU | |
| 1381887 | 1381888 | ACAGAGUCAGCCC | 577 | 1898 | 1920 | 1381889 | UUCUUUUGGGGC | 726 |
| | | CAAAAGAAUG | | | | | UGACUCUGU | |
| 1381892 | 1381895 | UCGUUUUCUGUGU | 578 | 1919 | 1941 | 1381894 | GCCAGCCAACAC | 727 |
| | | UGGCUGGCAC | | | | | AGAAAACGA | |
| 1381893 | 1381902 | CGGGCAUCAACAG | 579 | 1940 | 1962 | 1381903 | AGUUGAGCCUGU | 728 |
| | | GCUCAACUUC | | | | | UGAUGCCCG | |
| 1381896 | 1381898 | CCAGAACCUGGUC | 580 | 1982 | 2004 | 1381899 | GACCACUCGACC | 729 |
| | | GAGUGGUCAG | | | | | AGGUUCUGG | |
| 1381897 | 1381900 | AGUCCUCGGUCGG | 581 | 1961 | 1983 | 1381901 | CCCUGCUGCCGA | 730 |
| | | CAGCAGGGCG | | | | | CCGAGGACU | |
| 1381904 | 1381908 | CCGUAGUCAUGCA | 582 | 518 | 540 | 1381907 | UACCAACUUGCA | 731 |
| | | AGUUGGUACU | | | | | UGACUACGG | |
| 1381909 | 1381906 | AUUCCGCAGGGCA | 583 | 539 | 561 | 1381905 | CAUGUUGCUGCC | 732 |
| | | GCAACAUGCC | | | | | CUGCGGAAU | |
| 1381910 | 1381915 | UCUACCCCUCGGA | 584 | 560 | 582 | 1381916 | UGACAAGUUCCG | 733 |
| | | ACUUGUCAAU | | | | | AGGGGUAGA | |
| 1381911 | 1381914 | UCCGUCUUGAUAU | 585 | 2003 | 2025 | 1381913 | GUUGACAAAUAU | 734 |
| | | UUGUCAACCC | | | | | CAAGACGGA | |
| 1381912 | 1381919 | AUCUUCACUUCAG | 586 | 2024 | 2046 | 1381917 | GGAGAUCUCUGA | 735 |
| | | AGAUCUCCUC | | | | | AGUGAAGAU | |
| 1381918 | 1381921 | UCCACAUUGUCAC | 587 | 602 | 624 | 1381920 | UGAAGAAAGUG | 736 |
| | | UUUCUUCAGC | | | | | ACAAUGUGGA | |
| 1381922 | 1381924 | UCAUGUCGGAAUU | 588 | 2045 | 2067 | 1381926 | GGAUGCAGAAUU | 737 |
| | | CUGCAUCCAU | | | | | CCGACAUGA | |
| 1381923 | 1381925 | GCCAGUGGGCAAC | 589 | 581 | 603 | 1381927 | GUUUGUGUGUU | 738 |
| | | ACACAAACUC | | | | | GCCCACUGGC | |
| 1381928 | 1381932 | UGAUGAACUUCAU | 590 | 2066 | 2088 | 1381936 | CUCAGGAUAUGA | 739 |
| | | AUCCUGAGUC | | | | | AGUUCAUCA | |
| 1381929 | 1381934 | GCAAAGAACACCA | 591 | 2087 | 2109 | 1381931 | UCAAAAAUUGGU | 740 |
| | | AUUUUUGAUG | | | | | GUUCUUUGC | |
| 1381930 | 1381937 | UCCUCCUCCGCAU | 592 | 623 | 645 | 1381938 | UUCUGCUGAUGC | 741 |
| | | CAGCAGAAUC | | | | | GGAGGAGGA | |
| 1381933 | 1381941 | UUGUUUGAACCCA | 593 | 2108 | 2130 | 1381942 | AGAAGAUGUGG | 742 |
| | | CAUCUUCUGC | | | | | GUUCAAACAA | |
| 1381935 | 1381940 | CCCCACCAGACAU | 594 | 644 | 666 | 1381943 | UGACUCGGAUGU | 743 |
| | | CCGAGUCAUC | | | | | CUGGUGGGG | |
| 1381939 | 1381945 | AUGAGUCCAAUGA | 595 | 2129 | 2151 | 1381944 | AGGUGCAAUCAU | 744 |
| | | UUGCACCUUU | | | | | UGGACUCAU | |
| 1381946 | 1381955 | GCUAUGACAACAC | 596 | 2150 | 2172 | 1381951 | GGUGGGCGGUGU | 745 |
| | | CGCCCACCAU | | | | | UGUCAUAGC | |
| 1381949 | 1381954 | UGUUUCUUCUUCA | 597 | 2192 | 2214 | 1381947 | GGUGAUGCUGAA | 746 |
| | | GCAUCACCAA | | | | | GAAGAAACA | |
| 1381950 | 1381956 | AAGGUGAUGACGA | 598 | 2171 | 2193 | 1381957 | GACAGUGAUCGU | 747 |
| | | UCACUGUCGC | | | | | CAUCACCUU | |
| 1381953 | 1381952 | GCAUAGUCUGUGU | 599 | 665 | 687 | 1381948 | CGGAGCAGACAC | 748 |
| | | CUGCUCCGCC | | | | | AGACUAUGC | |
| 1381958 | 1381965 | CCAUGAUGAAUGG | 600 | 2213 | 2235 | 1381966 | GUACACAUCCAU | 749 |
| | | AUGUGUACUG | | | | | UCAUCAUGG | |
| 1381959 | 1381962 | GCGGCGUCAACCU | 601 | 2234 | 2256 | 1381961 | UGUGGUGGAGG | 750 |
| | | CCACCACACC | | | | | UUGACGCCGC | |
| 1381960 | 1381968 | UGGCGCUCCUCUG | 602 | 2255 | 2277 | 1381964 | UGUCACCCCAGA | 751 |
| | | GGGUGACAGC | | | | | GGAGCGCCA | |
| 1381963 | 1381971 | UAGGUUGGAUUU | 603 | 2297 | 2319 | 1381969 | CGGCUACGAAAA | 752 |
| | | UCGUAGCCGUU | | | | | UCCAACCUA | |
| 1381967 | 1381973 | ACUUUGUCUUCAC | 604 | 686 | 708 | 1381975 | AGAUGGGAGUG | 753 |
| | | UCCCAUCUGC | | | | | AAGACAAAGU | |
| 1381970 | 1381974 | UUCUGCUGCAUCU | 605 | 2276 | 2298 | 1381972 | CCUGUCCAAGAU | 754 |
| | | UGGACAGGUG | | | | | GCAGCAGAA | |
| 1381976 | 1381978 | UCCUCCUCUGCUA | 606 | 707 | 729 | 1381980 | AGUAGAAGUAGC | 755 |
| | | CUUCUACUAC | | | | | AGAGGAGGA | |
| 1381977 | 1381989 | UGCAUCUGCUCAA | 607 | 2318 | 2340 | 1381990 | CAAGUUCUUUGA | 756 |
| | | AGAACUUGUA | | | | | GCAGAUGCA | |
| 1381979 | 1381984 | GCUGUGGCGGGGG | 608 | 2339 | 2361 | 1381985 | GAACUAGACCCC | 757 |
| | | UCUAGUUCUG | | | | | CGCCACAGC | |
| 1381981 | 1381983 | UGCUGUCCAACUU | 609 | 2360 | 2382 | 1381987 | AGCCUCUGAAGU | 758 |
| | | CAGAGGCUGC | | | | | UGGACAGCA | |
| 1381982 | 1381991 | UCGUCAUCAUCGG | 610 | 749 | 771 | 1381986 | AGAAGAAGCCGA | 759 |
| | | cuucuucuuc | | | | | UGAUGACGA | |
| 1381988 | 1381992 | UCUUCCACCUCAG | 611 | 728 | 750 | 1381993 | AGAAGUGGCUGA | 760 |
| | | CCACUUCUUC | | | | | GGUGGAAGA | |
| 1381994 | 1381997 | UGGGUAGUGAAGC | 612 | 2381 | 2403 | 1381996 | AAACCAUUGCUU | 761 |
| | | AAUGGUUUUG | | | | | CACUACCCA | |
| 1381995 | 1382003 | UAUUCUAUAAAUG | 613 | 2402 | 2424 | 1382000 | UCGGUGUCCAUU | 762 |
| | | GACACCGAUG | | | | | UAUAGAAUA | |
| 1381998 | 1382002 | ACAGCACAGCUGU | 614 | 2465 | 2487 | 1382001 | GCCUUUUGACAG | 763 |
| | | CAAAAGGCGA | | | | | CUGUGCUGU | |
| 1381999 | 1382007 | GAUAAUGAGUAA | 615 | 2444 | 2466 | 1382005 | UUUUAUGAUUU | 764 |
| | | AUCAUAAAACG | | | | | ACUCAUUAUC | |
| 1382004 | 1382011 | CGGGUUUGUUUCU | 616 | 2423 | 2445 | 1382008 | AUGUGGGAAGA | 765 |
| | | UCCCACAUUA | | | | | AACAAACCCG | |
| 1382006 | 1382010 | GUUCAGGCAUCUA | 617 | 2486 | 2508 | 1382009 | AACACAAGUAGA | 766 |
| | | CUUGUGUUAC | | | | | UGCCUGAAC | |
| 1382012 | 1382013 | UCCUCAGCCUCUU | 618 | 791 | 813 | 1382014 | GGUAGAGGAAG | 767 |
| | | CCUCUACCUC | | | | | AGGCUGAGGA | |
| 1382015 | 1382017 | UCUGUGGCUUCUU | 619 | 812 | 834 | 1382018 | ACCCUACGAAGA | 768 |
| | | CGUAGGGUUC | | | | | AGCCACAGA | |
| 1382019 | 1382021 | AAAGAGAGAUAG | 620 | 2528 | 2550 | 1382016 | UAAUGUAUUCUA | 769 |
| | | AAUACAUUACU | | | | | UCUCUCUUU | |
| 1382020 | 1382024 | CUGAUGUGUGGAU | 621 | 2507 | 2529 | 1382022 | UUGAAUUAAUCC | 770 |
| | | UAAUUCAAGU | | | | | ACACAUCAG | |
| 1382023 | 1382029 | GUGGCAAUGCUGG | 622 | 833 | 855 | 1382028 | GAGAACCACCAG | 771 |
| | | UGGUUCUCUC | | | | | CAUUGCCAC | |
| 1382025 | 1382027 | GUAGUAUAGAGAC | 623 | 2549 | 2571 | 1382026 | ACAUUUUGGUCU | 772 |
| | | CAAAAUGUAA | | | | | CUAUACUAC | |
| 1382030 | 1382032 | UCAUCACCAUCCU | 624 | 770 | 792 | 1382031 | GGACGAUGAGGA | 773 |
| | | CAUCGUCCUC | | | | | UGGUGAUGA | |
| 1382033 | 1382038 | UACACAAAACCCA | 625 | 2570 | 2592 | 1382035 | AUUAUUAAUGG | 774 |
| | | UUAAUAAUGU | | | | | GUUUUGUGUA | |
| 1382034 | 1382037 | AUACAGCUAAAUU | 626 | 2591 | 2613 | 1382042 | CUGUAAAGAAUU | 775 |
| | | CUUUACAGUA | | | | | UAGCUGUAU | |
| 1382036 | 1382041 | UCUGUGGUGGUGG | 627 | 854 | 876 | 1382040 | CACCACCACCAC | 776 |
| | | UGGUGGUGGU | | | | | CACCACAGA | |
| 1382039 | 1382046 | AUCUAUUCAUGCA | 628 | 2612 | 2634 | 1382047 | CAAACUAGUGCA | 777 |
| | | CUAGUUUGAU | | | | | UGAAUAGAU | |
| 1382043 | 1382044 | CGAACCACCUCUU | 629 | 875 | 897 | 1382045 | GUCUGUGGAAGA | 778 |
| | | CCACAGACUC | | | | | GGUGGUUCG | |
| 1382051 | 1382064 | GUGAUAAAUAAUC | 630 | 2633 | 2655 | 1382060 | UCUCUCCUGAUU | 779 |
| | | AGGAGAGAAU | | | | | AUUUAUCAC | |
| 1382052 | 1382058 | UCACAAACCACAA | 631 | 2675 | 2697 | 1382055 | UAUUAUUCUUGU | 780 |
| | | GAAUAAUAUA | | | | | GGUUUGUGA | |
| 1382053 | 1382057 | UACAACUGGCUAA | 632 | 2654 | 2676 | 1382056 | AUAGCCCCUUAG | 781 |
| | | GGGGCUAUGU | | | | | CCAGUUGUA | |
| 1382054 | 1382061 | GUAAAGUAGGACU | 633 | 2696 | 2718 | 1382062 | CCCAAUUAAGUC | 782 |
| | | UAAUUGGGUC | | | | | CUACUUUAC | |
| 1382059 | 1382068 | CCAUCGAUUCUUA | 634 | 2717 | 2739 | 1382065 | AUAUGCUUUAAG | 783 |
| | | AAGCAUAUGU | | | | | AAUCGAUGG | |
| 1382063 | 1382067 | CACGUUCACAUGA | 635 | 2738 | 2760 | 1382066 | GGGAUGCUUCAU | 784 |
| | | AGCAUCCCCC | | | | | GUGAACGUG | |
| 1382069 | 1382076 | CAAGAGAAGCAGC | 636 | 2759 | 2781 | 1382072 | GGAGUUCAGCUG | 785 |
| | | UGAACUCCCA | | | | | CUUCUCUUG | |
| 1382070 | 1382074 | AUCAGGAAAGGAA | 637 | 2780 | 2802 | 1382073 | CCUAAGUAUUCC | 786 |
| | | UACUUAGGCA | | | | | UUUCCUGAU | |
| 1382071 | 1382077 | AUCUGAAAUACUU | 638 | 2822 | 2844 | 1382079 | ACAUUUUUAAGU | 787 |
| | | AAAAAUGUUU | | | | | AUUUCAGAU | |
| 1382075 | 1382081 | UUAACUUUAAAAU | 639 | 2801 | 2823 | 1382082 | CACUAUGCAUUU | 788 |
| | | GCAUAGUGAU | | | | | UAAAGUUAA | |
| 1382078 | 1382085 | GAAAAAAAAUCUC | 640 | 2843 | 2865 | 1382083 | GCUUUAGAGAGA | 789 |
| | | UCUAAAGCAU | | | | | UUUUUUUUC | |
| 1382080 | 1382086 | GUACAGUAAAAUG | 641 | 2864 | 2886 | 1382084 | CAUGACUGCAUU | 790 |
| | | CAGUCAUGGA | | | | | UUACUGUAC | |
| 1382087 | 1382095 | AUAUAGCAGAAGC | 642 | 2885 | 2907 | 1382098 | AGAUUGCUGCUU | 791 |
| | | AGCAAUCUGU | | | | | CUGCUAUAU | |
| 1382088 | 1382092 | CUCUUAAUUCCUA | 643 | 2906 | 2928 | 1382091 | UUGUGAUAUAG | 792 |
| | | UAUCACAAAU | | | | | GAAUUAAGAG | |
| 1382089 | 1382093 | GAAGAAACAAACG | 644 | 2927 | 2949 | 1382097 | GAUACACACGUU | 793 |
| | | UGUGUAUCCU | | | | | UGUUUCUUC | |
| 1382090 | 1382096 | GUGUGCACAUAAA | 645 | 2948 | 2970 | 1382100 | GUGCCUGUUUUA | 794 |
| | | ACAGGCACGA | | | | | UGUGCACAC | |
| 1382103 | 1382101 | CUUGAAGUCUCAA | 646 | 2969 | 2991 | 1382102 | AUUAGGCAUUGA | 795 |
| | | UGCCUAAUGU | | | | | GACUUCAAG | |
| 1382104 | 1382099 | ACGUGGACAAAAA | 647 | 2990 | 3012 | 1382094 | CUUUUCUUUUUU | 796 |
| | | AAGAAAAGCU | | | | | UGUCCACGU | |
| 1382105 | 1382110 | CUUUAUCAAAGAC | 648 | 3011 | 3033 | 1382118 | AUCUUUGGGUCU | 797 |
| | | CCAAAGAUAC | | | | | UUGAUAAAG | |
| 1382106 | 1382112 | ACCAGCAGAGCAC | 649 | 3074 | 3096 | 1382108 | GGGGAGGGGUGC | 798 |
| | | CCCUCCCCAC | | | | | UCUGCUGGU | |
| 1382107 | 1382111 | ACCCGCCCCGUAA | 650 | 3053 | 3075 | 1382114 | AAGCACUUUUAC | 799 |
| | | AAGUGCUUAC | | | | | GGGGCGGGU | |
| 1382116 | 1382113 | ACAAUGAACAGGG | 651 | 3032 | 3054 | 1382109 | AAAAGAAUCCCU | 800 |
| | | AUUCUUUUCU | | | | | GUUCAUUGU | |
| 1382119 | 1382115 | GAGAAUUCUUGGU | 652 | 3095 | 3117 | 1382117 | CUUCAAUUACCA | 801 |
| | | AAUUGAAGAC | | | | | AGAAUUCUC | |
| 1382207 | 1382216 | CCUCUCGAACCAC | 653 | 880 | 902 | 1382215 | UGGAAGAGGUG | 802 |
| | | CUCUUCCACA | | | | | GUUCGAGAGG | |
| 1382208 | 1382210 | UCUCGGCUUGUUC | 654 | 901 | 923 | 1382211 | UGUGCUCUGAAC | 803 |
| | | AGAGCACACC | | | | | AAGCCGAGA | |
| 1382212 | 1382217 | UCAUUGCUCGGCA | 655 | 922 | 944 | 1382218 | CGGGGCCGUGCC | 804 |
| | | CGGCCCCGUC | | | | | GAGCAAUGA | |
| 1382222 | 1382230 | CAUCAAAGUACCA | 656 | 943 | 965 | 1382232 | UCUCCCGCUGGU | 805 |
| | | GCGGGAGAUC | | | | | ACUUUGAUG | |
| 1382229 | 1382235 | GGGCACACUUCCC | 657 | 964 | 986 | 1382236 | UGACUGAAGGGA | 806 |
| | | UUCAGUCACA | | | | | AGUGUGCCC | |
| 1382237 | 1382241 | CCAUGCAGUACUC | 658 | 1027 | 1049 | 1382242 | ACACAGAAGAGU | 807 |
| | | UUCUGUGUCA | | | | | ACUGCAUGG | |
| 1382239 | 1382244 | CACAUCCGCCGUA | 659 | 985 | 1007 | 1382245 | CAUUCUUUUACG | 808 |
| | | AAAGAAUGGG | | | | | GCGGAUGUG | |
| 1382240 | 1382249 | ACAUGGCGCUGCC | 660 | 1048 | 1070 | 1382248 | CCGUGUGUGGCA | 809 |
| | | ACACACGGCC | | | | | GCGCCAUGU | |
| 1382243 | 1382238 | CAAAGUUGUUCCG | 661 | 1006 | 1028 | 1382247 | GCGGCAACCGGA | 810 |
| | | GUUGCCGCCA | | | | | ACAACUUUG | |
| 1382256 | 1382263 | CUCGGGCAAGAGG | 662 | 1090 | 1112 | 1382260 | CCCAGGAACCUC | 811 |
| | | UUCCUGGGUA | | | | | UUGCCCGAG | |
| 1382259 | 1382261 | UAGUCUUGAGUAA | 663 | 1069 | 1091 | 1382262 | CCCAAAGUUUAC | 812 |
| | | ACUUUGGGAC | | | | | UCAAGACUA | |
| 1382269 | 1382266 | ACUGGCUGCUGUU | 664 | 1122 | 1144 | 1382267 | CUUCCUACAACA | 813 |
| | | GUAGGAAGUU | | | | | GCAGCCAGU | |
| 1382273 | 1382276 | UUGUAGGAAGUU | 665 | 1111 | 1133 | 1382275 | AUCCUGUUAAAC | 814 |
| | | UAACAGGAUCU | | | | | UUCCUACAA | |

TABLE 15
RNAi compounds targeting human APP SEQ ID No: 3

| | | | | SEQ ID | SEQ ID | | | |
|---|---|---|---|---|---|---|---|---|
| | | Antisense | SEQ | NO: 3 | NO: 3 | | | |
| Compound | Antisense | Sequence | ID | Antisense | Antisense | Sense | Sense Sequence | SEQ |
| Number | oligo ID | (5′ to 3′) | NO | Start Site | Stop Site | oligo ID | (5′ to 3′) | ID NO |
| 1376142 | 1378900 | GCCGUCUCCCGGG | 815 | 63 | 85 | 1378899 | GCGGGGGCCCC | 841 |
| | | GCCCCCGCGC | | | | | GGGAGACGGC | |
| 1376283 | 1376285 | CGCCUACCGCUGC | 816 | 21 | 43 | 1378828 | UUUCCUCGGCA | 842 |
| | | CGAGGAAACU | | | | | GCGGUAGGCG | |
| 1378827 | 1378829 | GCACGCUCCUCCG | 817 | 42 | 64 | 1378830 | AGAGCACGCGG | 843 |
| | | CGUGCUCUCG | | | | | AGGAGCGUGC | |
| 1378897 | 1378901 | UCUGCCCGCGCCG | 818 | 84 | 106 | 1378898 | GGCGGUGGCGG | 844 |
| | | CCACCGCCGC | | | | | CGCGGGCAGA | |
| 1381703 | 1381705 | UGGGAUCCGCCG | 819 | 105 | 127 | 1381704 | GCAAGGACGCG | 845 |
| | | CGUCCUUGCUC | | | | | GCGGAUCCCA | |
| 1381706 | 1381708 | CCGAGUGCGCUG | 820 | 126 | 148 | 1381707 | CUCGCACAGCA | 846 |
| | | CUGUGCGAGUG | | | | | GCGCACUCGG | |
| 1382120 | 1382121 | AUCCUGCAGAAA | 821 | 3192 | 3214 | 1382122 | CAAAACAAUUU | 847 |
| | | AUUGUUUUGGA | | | | | UCUGCAGGAU | |
| 1382123 | 1382129 | UAAUUUAUUUAU | 822 | 3255 | 3277 | 1382126 | CUGUAUUACAU | 848 |
| | | GUAAUACAGUG | | | | | AAAUAAAUUA | |
| 1382124 | 1382131 | UGUAGAAAGCGA | 823 | 3234 | 3256 | 1382125 | AUGACAUGAUC | 849 |
| | | UCAUGUCAUAA | | | | | GCUUUCUACA | |
| 1382128 | 1382134 | AAGCAAUGAUUC | 824 | 3213 | 3235 | 1382127 | GAUUGUACAGA | 850 |
| | | UGUACAAUCAU | | | | | AUCAUUGCUU | |
| 1382130 | 1382136 | CUUGCCCGGGGU | 825 | 3276 | 3298 | 1382135 | AAUAAAAUAAC | 851 |
| | | UAUUUUAUUUA | | | | | CCCGGGCAAG | |
| 1382132 | 1382137 | AGUCAUCCUUCA | 826 | 3297 | 3319 | 1382139 | ACUUUUCUUUG | 852 |
| | | AAGAAAAGUCU | | | | | AAGGAUGACU | |
| 1382133 | 1382138 | CUUCGAUUAUUU | 827 | 3318 | 3340 | 1382140 | ACAGACAUUAA | 853 |
| | | AAUGUCUGUAG | | | | | AUAAUCGAAG | |
| 1382141 | 1382145 | UUAAAGAAAAUU | 828 | 3360 | 3382 | 1382146 | GGCAGAUUCAA | 854 |
| | | GAAUCUGCCUC | | | | | UUUUCUUUAA | |
| 1382142 | 1382148 | UCUUCUCCCCACC | 829 | 3339 | 3361 | 1382143 | UAAUUUUGGGU | 855 |
| | | CAAAAUUACU | | | | | GGGGAGAAGA | |
| 1382144 | 1382150 | AUUUUCAUCUUC | 830 | 3402 | 3424 | 1382149 | GAUACAAAAGA | 856 |
| | | UUUUGUAUCAU | | | | | AGAUGAAAAU | |
| 1382147 | 1382153 | AUAAAUGAAACU | 831 | 3381 | 3403 | 1382155 | CCAGUCUGAAG | 857 |
| | | UCAGACUGGUU | | | | | UUUCAUUUAU | |
| 1382151 | 1382154 | UGUCCAGGCAUG | 832 | 3444 | 3466 | 1382157 | UGAGGAAGGCA | 858 |
| | | CCUUCCUCAUC | | | | | UGCCUGGACA | |
| 1382152 | 1382156 | UCCCCUUAUAUU | 833 | 3423 | 3445 | 1382158 | GGAAGUGGCAA | 859 |
| | | GCCACUUCCAU | | | | | UAUAAGGGGA | |
| 1382159 | 1382164 | ACACAUCUUAAA | 834 | 3465 | 3487 | 1382160 | AACCCUUCUUU | 860 |
| | | AGAAGGGUUUG | | | | | UAAGAUGUGU | |
| 1382161 | 1382168 | UGUAUUUAUUUA | 835 | 3507 | 3529 | 1382167 | GUUUUCAUGUA | 861 |
| | | CAUGAAAACAC | | | | | AAUAAAUACA | |
| 1382162 | 1382166 | ACCAUUUUAUAC | 836 | 3486 | 3508 | 1382165 | CUUCAAUUUGU | 862 |
| | | AAAUUGAAGAC | | | | | AUAAAAUGGU | |
| 1382163 | 1382169 | UGCUCCUCCAAG | 837 | 3521 | 3543 | 1382170 | AAAUACAUUCU | 863 |
| | | AAUGUAUUUAU | | | | | UGGAGGAGCA | |
| 1382270 | 1382271 | GAAUGGCGCUGC | 838 | 1181 | 1203 | 1382272 | CCGUGUGUGGC | 864 |
| | | CACACACGGCC | | | | | AGCGCCAUUC | |
| 1382274 | 1382277 | UACUGGCUGCUG | 839 | 1199 | 1221 | 1382197 | UUCCUACAACA | 865 |
| | | UUGUAGGAAUG | | | | | GCAGCCAGUA | |
| 1382278 | 1382279 | ACUGACGGAGCC | 840 | 1 | 23 | 1382280 | CCGCGCUCGGGC | 866 |
| | | CGAGCGCGGCG | | | | | UCCGUCAGU | |

TABLE 16
RNAi targeting human APP SEQ ID No: 4

| | | | | SEQ ID | SEQ ID | | | |
|---|---|---|---|---|---|---|---|---|
| | | Antisense | SEQ | NO: 4 | NO: 4 | | | |
| Compound | Antisense | Sequence | ID | Antisense | Antisense | Sense | Sense Sequence | SEQ |
| Number | oligo ID | (5′ to 3′) | NO | Start Site | Stop Site | oligo ID | (5′ to 3′) | ID NO |
| 1382173 | 1382178 | GGAACUCGAACC | 867 | 994 | 1016 | 1382177 | GGAAGAGGUGGU | 889 |
| | | ACCUCUUCCAC | | | | | UCGAGUUCC | |
| 1382172 | 1382179 | UAGGAACUCGAA | 868 | 996 | 1018 | 1382174 | AAGAGGUGGUUC | 890 |
| | | CCACCUCUUCC | | | | | GAGUUCCUA | |
| 1382175 | 1382183 | AACUCGAACCACC | 869 | 992 | 1014 | 1382180 | GUGGAAGAGGUG | 891 |
| | | UCUUCCACAG | | | | | GUUCGAGUU | |
| 1382176 | 1382182 | AGGAACUCGAAC | 870 | 995 | 1017 | 1382184 | GAAGAGGUGGUU | 892 |
| | | CACCUCUUCCA | | | | | CGAGUUCCU | |
| 1382181 | 1382185 | GAACUCGAACCA | 871 | 993 | 1015 | 1382188 | UGGAAGAGGUGG | 893 |
| | | CCUCUUCCACA | | | | | UUCGAGUUC | |
| 1382171 | 1382186 | ACUCGAACCACCU | 872 | 991 | 1013 | 1382187 | UGUGGAAGAGGU | 894 |
| | | CUUCCACAGA | | | | | GGUUCGAGU | |
| 1382191 | 1382194 | UACUGGCUGCUG | 873 | 1011 | 1033 | 1382197 | UUCCUACAACAG | 865 |
| | | UUGUAGGAACU | | | | | CAGCCAGUA | |
| 1382190 | 1382199 | UUGUAGGAACUC | 874 | 999 | 1021 | 1382192 | AGGUGGUUCGAG | 895 |
| | | GAACCACCUCU | | | | | UUCCUACAA | |
| 1382193 | 1382198 | ACUGGCUGCUGU | 875 | 1010 | 1032 | 1382200 | GUUCCUACAACA | 896 |
| | | UGUAGGAACUC | | | | | GCAGCCAGU | |
| 1382189 | 1382195 | GUAGGAACUCGA | 876 | 997 | 1019 | 1382196 | AGAGGUGGUUCG | 897 |
| | | ACCACCUCUUC | | | | | AGUUCCUAC | |
| 1382206 | 1382204 | GUACUGGCUGCU | 877 | 1012 | 1034 | 1382201 | UCCUACAACAGC | 898 |
| | | GUUGUAGGAAC | | | | | AGCCAGUAC | |
| 1382205 | 1382203 | GUUGUAGGAACU | 878 | 1000 | 1022 | 1382202 | GGUGGUUCGAGU | 899 |
| | | CGAACCACCUC | | | | | UCCUACAAC | |
| 1382209 | 1382214 | CUGUUGUAGGAA | 879 | 1002 | 1024 | 1382213 | UGGUUCGAGUUC | 900 |
| | | CUCGAACCACC | | | | | CUACAACAG | |
| 1382219 | 1382223 | UGUAGGAACUCG | 880 | 998 | 1020 | 1382221 | GAGGUGGUUCGA | 901 |
| | | AACCACCUCUU | | | | | GUUCCUACA | |
| 1382220 | 1382226 | UGUUGUAGGAAC | 881 | 1001 | 1023 | 1382224 | GUGGUUCGAGUU | 902 |
| | | UCGAACCACCU | | | | | CCUACAACA | |
| 1382228 | 1382234 | UGCUGUUGUAGG | 882 | 1004 | 1026 | 1382233 | GUUCGAGUUCCU | 903 |
| | | AACUCGAACCA | | | | | ACAACAGCA | |
| 1382225 | 1382231 | GCUGUUGUAGGA | 883 | 1003 | 1025 | 1382227 | GGUUCGAGUUCC | 904 |
| | | ACUCGAACCAC | | | | | UACAACAGC | |
| 1382250 | 1382251 | CUGCUGUUGUAG | 884 | 1005 | 1027 | 1382253 | UUCGAGUUCCUA | 905 |
| | | GAACUCGAACC | | | | | CAACAGCAG | |
| 1382246 | 1382254 | GGCUGCUGUUGU | 885 | 1007 | 1029 | 1382252 | CGAGUUCCUACA | 906 |
| | | AGGAACUCGAA | | | | | ACAGCAGCC | |
| 1382255 | 1382257 | UGGCUGCUGUUG | 886 | 1008 | 1030 | 1382258 | GAGUUCCUACAA | 907 |
| | | UAGGAACUCGA | | | | | CAGCAGCCA | |
| 1382268 | 1382265 | GCUGCUGUUGUA | 887 | 1006 | 1028 | 1382264 | UCGAGUUCCUAC | 908 |
| | | GGAACUCGAAC | | | | | AACAGCAGC | |
| 1382048 | 1382050 | CUGGCUGCUGUU | 888 | 1009 | 1031 | 1382049 | AGUUCCUACAAC | 909 |
| | | GUAGGAACUCG | | | | | AGCAGCCAG | |

### Example 5: Effect of RNAi Compounds on Human APP RNA In Vitro, Single Dose

Double-stranded RNAi compounds described above were tested in a series of experiments under the same culture conditions. The results for each experiment are presented in separate tables below.

Cultured HeLa cells at a density of 6000 cells per well were transfected using RNAiMAX with 20 nM of double-stranded RNAi. After a treatment period of approximately 24 hours, RNA was isolated from the cells and APP RNA levels were measured by quantitative real-time RTPCR. Human primer probe set RTS35571 (described herein above) was used to measure RNA levels. APP RNA levels were normalized to total RNA content, as measured by RIBOGREEN®. Results are presented as percent change of APP RNA, relative to PBS control. The symbol “f” indicates that the modified oligonucleotide is complementary to the target transcript within the amplicon region of the primer probe set and so the associated data is not reliable. In such instances, additional assays using alternative primer probes must be performed to accurately assess the potency and efficacy of such modified oligonucleotides.

TABLE 17
Reduction of APP RNA by RNAi

| Compound | APP RNA |
|---|---|
| Number | (% control) |
| 1376283 | 96 |
| 1378827 | 102 |
| 1376142 | 135 |
| 1378897 | 96 |
| 1381703 | 59 |
| 1381706 | 40 |
| 1381710 | 81 |
| 1381709 | 18 |
| 1381712 | 92 |
| 1381734 | 91 |
| 1381733 | 33 |
| 1381735 | 8 |
| 1381736 | 38 |
| 1381740 | 17 |
| 1381755 | 7 |
| 1381771 | 20 |
| 1381772 | 8 |
| 1381778 | 11 |
| 1381773 | 30 |
| 1381776 | 18 |
| 1381789 | 24 |
| 1381790 | 35 |
| 1381798 | 50 |
| 1381809 | 26 |
| 1381810 | 33 |
| 1381817 | 70 |
| 1381818 | 16 |
| 1381825 | 10 |
| 1381830 | 88 |
| 1381832 | 8 |
| 1381835 | 56 |
| 1381842 | 58 |
| 1381909 | 76 |
| 1381904 | 3 |
| 1381910 | 90 |
| 1381918 | 9 |
| 1381923 | 20 |
| 1381930 | 85 |
| 1381935 | 76 |
| 1381953 | 21 |
| 1381982 | 14 |
| 1381988 | 25 |
| 1382030 | 15 |
| 1382173 | 88 |
| 1382172 | 94 |
| 1382176 | 89 |
| 1382175 | 49 |
| 1382181 | 82 |
| 1382171 | 26 |
| 1382191 | 28 |
| 1382189 | 88 |
| 1382193 | 105 |
| 1382190 | 87 |
| 1382205 | 99 |
| 1382206 | 75 |
| 1382208 | 27 |
| 1382209 | 83 |
| 1382207 | 75 |
| 1382212 | 81 |
| 1382219 | 66 |
| 1382220 | 87 |
| 1382222 | 58 |
| 1382225 | 108 |
| 1382228 | 109 |
| 1382229 | 40 |
| 1382243 | 28 |
| 1382237 | 28 |
| 1382239 | 63 |
| 1382240 | 60 |
| 1382250 | 86 |
| 1382246 | 101 |
| 1382255 | 96 |
| 1382259 | 55 |
| 1382256 | 83 |
| 1382268 | 100 |
| 1382269 | 64 |
| 1382270 | 66 |
| 1382273 | 51 |
| 1382274 | 47 |
| 1382278 | 93 |

TABLE 18
Reduction of APP RNA by RNAi

| Compound | APP RNA |
|---|---|
| Number | (% control) |
| 1381718 | 51 |
| 1381721 | 53 |
| 1381723 | 61 |
| 1381725 | 51 |
| 1381727 | 98 |
| 1381746 | 66 |
| 1381751 | 15 |
| 1381756 | 9 |
| 1381752 | 8 |
| 1381761 | 68 |
| 1381759 | 50 |
| 1381782 | 21 |
| 1381791 | 32 |
| 1381792 | 76 |
| 1381795 | 90 |
| 1381802 | 66 |
| 1381808 | 98 |
| 1381826 | 9 |
| 1381828 | 72 |
| 1381840 | 29 |
| 1381844 | 63 |
| 1381850 | 45 |
| 1381853 | 7 |
| 1381854 | 71 |
| 1381858 | 23 |
| 1381855 | 44 |
| 1381861 | 8 |
| 1381864 | 13 |
| 1381872 | 42 |
| 1381874 | 6 |
| 1381875 | 56 |
| 1381876 | 8 |
| 1381879 | 72 |
| 1381887 | 82 |
| 1381882 | 32 |
| 1381892 | 5 |
| 1381896 | 89 |
| 1381897 | 73 |
| 1381893 | 17 |
| 1381911 | 19 |
| 1381912 | 17 |
| 1381922 | 9 |
| 1381928 | 10 |
| 1381929 | 14 |
| 1381933 | 16 |
| 1381939 | 7 |
| 1381949 | 13 |
| 1381946 | 49 |
| 1381950 | 37 |
| 1381959 | 62 |
| 1381958 | 8 |
| 1381960 | 97 |
| 1381963 | 12 |
| 1381967 | 13 |
| 1381970 | 8 |
| 1381976 | 70 |
| 1381981 | 13 |
| 1381979 | 97 |
| 1381977 | 16 |
| 1381994 | 12 |
| 1381998 | 13 |
| 1381995 | 9 |
| 1381999 | 8 |
| 1382006 | 10 |
| 1382004 | 8 |
| 1382012 | 35 |
| 1382015 | 60 |
| 1382019 | 7 |
| 1382020 | 11 |
| 1382025 | 8 |
| 1382023 | 38 |
| 1382034 | 6 |
| 1382033 | 10 |
| 1382036 | 89 |
| 1382043 | 62 |
| 1382039 | 17 |
| 1382048 | 89 |
| 1382053 | 38 |
| 1382051 | 9 |
| 1382059 | 8 |

TABLE 19
Reduction of APP RNA by RNAi

| Compound | APP RNA |
|---|---|
| Number | (% control) |
| 1382052 | 12 |
| 1382054 | 5 |
| 1382063 | 16 |
| 1382070 | 10 |
| 1382069 | 10 |
| 1382071 | 10 |
| 1382075 | 7 |
| 1382078 | 11 |
| 1382080 | 13 |
| 1382088 | 14 |
| 1382089 | 5 |
| 1382087 | 9 |
| 1382090 | 7 |
| 1382104 | 4 |
| 1382103 | 6 |
| 1382105 | 5 |
| 1382107 | 30 |
| 1382106 | 41 |
| 1382116 | 7 |
| 1382119 | 7 |
| 1382120 | 8 |
| 1382123 | 5 |
| 1382124 | 7 |
| 1382128 | 5 |
| 1382130 | 67 |
| 1382132 | 51 |
| 1382133 | 57 |
| 1382141 | 60 |
| 1382142 | 67 |
| 1382144 | 68 |
| 1382147 | 56 |
| 1382151 | 66 |
| 1382152 | 62 |
| 1382159 | 62 |
| 1382162 | 54 |
| 1382161 | 51 |
| 1382163 | 64 |

### Example 6: Activity of Modified Oligonucleotides Complementary to Human APP in Transgenic Mice

Compounds described above are tested in the Tc1 transgenic mouse model which contains a freely segregating, almost complete human chromosome 21 (Hsa21) with 92% of all known Hsa21 genes including APP (O'Doherty et al., Science 2005 309(5743):2033-2037). Compounds are also tested in the R1.40 YAC transgenic mouse model which contains the entire human APP gene harboring the Swedish mutations (K670N/M671L) as described in Lamb et al., Human Mol Genetics 1997, 6(9):1535-41. Groups of 2-3 mice are injected ICV with 300 ug ASO or PBS control, and sacrificed at 2 weeks post dosing. Various CNS tissues are collected. APP RNA are measured by RT-PCR as described in Example 1 above.

1.-4. (canceled)

5. An oligomeric compound comprising a modified oligonucleotide consisting of 12 to 30 linked nucleosides wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, or at least 21 contiguous nucleobases of:
an equal length portion of nucleobases 40-78 of SEQ ID NO: 1;
an equal length portion of nucleobases 69-146 of SEQ ID NO: 1;
an equal length portion of nucleobases 83-129 of SEQ ID NO: 1;
an equal length portion of nucleobases 83-246 of SEQ ID NO: 1;
an equal length portion of nucleobases 94-225 of SEQ ID NO: 1;
an equal length portion of nucleobases 194-231 of SEQ ID NO: 1;
an equal length portion of nucleobases 194-238 of SEQ ID NO: 1;
an equal length portion of nucleobases 236-268 of SEQ ID NO: 1;
an equal length portion of nucleobases 258-288 of SEQ ID NO: 1;
an equal length portion of nucleobases 285-311 of SEQ ID NO: 1;
an equal length portion of nucleobases 296-321 of SEQ ID NO: 1;
an equal length portion of nucleobases 307-330 of SEQ ID NO: 1;
an equal length portion of nucleobases 329-352 of SEQ ID NO: 1;
an equal length portion of nucleobases 330-352 of SEQ ID NO: 1;
an equal length portion of nucleobases 339-383 of SEQ ID NO: 1;
an equal length portion of nucleobases 415-439 of SEQ ID NO: 1;
an equal length portion of nucleobases 413-477 of SEQ ID NO: 1;
an equal length portion of nucleobases 415-477 of SEQ ID NO: 1;
an equal length portion of nucleobases 477-506 of SEQ ID NO: 1;
an equal length portion of nucleobases 477-523 of SEQ ID NO: 1;
an equal length portion of nucleobases 477-541 of SEQ ID NO: 1;
an equal length portion of nucleobases 530-557 of SEQ ID NO: 1;
an equal length portion of nucleobases 581-638 of SEQ ID NO: 1;
an equal length portion of nucleobases 636-661 of SEQ ID NO: 1;
an equal length portion of nucleobases 652-697 of SEQ ID NO: 1;
an equal length portion of nucleobases 728-821 of SEQ ID NO: 1;
an equal length portion of nucleobases 770-821 of SEQ ID NO: 1;
an equal length portion of nucleobases 920-950 of SEQ ID NO: 1;
an equal length portion of nucleobases 1006-1049 of SEQ ID NO: 1;
an equal length portion of nucleobases 1152-1179 of SEQ ID NO: 1;
an equal length portion of nucleobases 1227-1265 of SEQ ID NO: 1;
an equal length portion of nucleobases 1227-1274 of SEQ ID NO: 1;
an equal length portion of nucleobases 1268-1332 of SEQ ID NO: 1;
an equal length portion of nucleobases 1268-1311 of SEQ ID NO: 1;
an equal length portion of nucleobases 1289-1332 of SEQ ID NO: 1;
an equal length portion of nucleobases 1518-1543 of SEQ ID NO: 1;
an equal length portion of nucleobases 1531-1593 of SEQ ID NO: 1;
an equal length portion of nucleobases 1544-1593 of SEQ ID NO: 1;
an equal length portion of nucleobases 1634-1657 of SEQ ID NO: 1;
an equal length portion of nucleobases 1778-1800 of SEQ ID NO: 1;
an equal length portion of nucleobases 1882-1908 of SEQ ID NO: 1;
an equal length portion of nucleobases 2051-2074 of SEQ ID NO: 1;
an equal length portion of nucleobases 2360-3117 of SEQ ID NO: 1;
an equal length portion of nucleobases 2402-3117 of SEQ ID NO: 1;
an equal length portion of nucleobases 2360-2655 of SEQ ID NO: 1;
an equal length portion of nucleobases 2402-2655 of SEQ ID NO: 1;
an equal length portion of nucleobases 2675-3054 of SEQ ID NO: 1; or
an equal length portion of nucleobases 3192-3277 of SEQ ID NO: 3;
wherein the modified oligonucleotide comprises at least one modification selected from a modified sugar and a modified internucleoside linkage.

6. The oligomeric compound of claim 5, wherein the nucleobase sequence of the modified oligonucleotide is at least 80%, at least 85%, at least 90%, at least 95%, or 100% complementary to the nucleobase sequences of any of SEQ ID NO: 1-7 when measured across the entire nucleobase sequence of the modified oligonucleotide.

7. The oligomeric compound of claim 5, wherein at least one nucleoside of the modified oligonucleotide is a modified nucleoside.

8. The oligomeric compound of claim 7, wherein at least one modified nucleoside of the modified oligonucleotide comprises a modified sugar moiety.

9. The oligomeric compound of claim 8, wherein at least one modified nucleoside of the modified oligonucleotide comprises a bicyclic sugar moiety.

10. The oligomeric compound of claim 9, wherein at least one modified nucleoside of the modified oligonucleotide comprises a bicyclic sugar moiety having a 2′-4′ bridge, wherein the 2′-4′ bridge is selected from —O—CH₂— and —O—CH(CH₃)—.

11. The oligomeric compound of claim 7, wherein at least one modified nucleoside of the modified oligonucleotide comprises a non-bicyclic modified sugar moiety.

12. The oligomeric compound of claim 11, wherein at least one modified nucleoside of the modified oligonucleotide comprises a bicyclic sugar moiety having a 2′-4′ bridge and at least one modified nucleoside of the modified oligonucleotide comprises a non-bicyclic modified sugar moiety.

13. The oligomeric compound of claim 11, wherein the non-bicyclic modified sugar moiety is a 2′-MOE modified sugar moiety, a 2′-OMe modified sugar moiety, or a 2′-F modified sugar moiety.

14. The oligomeric compound of claim 5, wherein the modified oligonucleotide comprises at least one modified nucleoside comprising a sugar surrogate.

15. The oligomeric compound of claim 14, wherein at least one modified nucleoside of the modified oligonucleotide comprises a sugar surrogate selected from morpholino and PNA.

16. The oligomeric compound of claim 5, wherein the modified oligonucleotide comprises at least one modified internucleoside linkage.

17. The oligomeric compound of claim 16, wherein each internucleoside linkage of the modified oligonucleotide is a modified internucleoside linkage.

18. The oligomeric compound of claim 16, wherein at least one modified internucleoside linkage is a phosphorothioate internucleoside linkage.

19. The oligomeric compound of claim 16, wherein the modified oligonucleotide comprises at least one phosphodiester internucleoside linkage.

20. The oligomeric compound of claim 16, wherein each internucleoside linkage of the modified oligonucleotide is independently selected from a phosphodiester internucleoside linkage or a phosphorothioate internucleoside linkage.

21. The oligomeric compound of claim 5, wherein the modified oligonucleotide comprises at least one modified nucleobase.

22. The oligomeric compound of claim 21, wherein the modified nucleobase is a 5-methyl cytosine.

23. (canceled)

24. The oligomeric compound of claim 5, wherein the modified oligonucleotide consists of 18-21 linked nucleosides.

25.-28. (canceled)

29. The oligomeric compound of claim 5, wherein the modified oligonucleotide is a gapmer.

30. The oligomeric compound of claim 5, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides;
wherein the 3′-most nucleoside of the 5′-region and the 5′-most nucleoside of the 3′-region comprise modified sugar moieties, and
each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprising a 2′-substituted sugar moiety.

31. The oligomeric compound of claim 5, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and each of the central region nucleosides comprises a 2′-β-D-deoxyribosyl sugar moiety.

32. (canceled)

33. (canceled)

34. The oligomeric compound of claim 5, wherein the oligomeric compound comprises an antisense RNAi oligonucleotide comprising a targeting region comprising at least 15, 19, 20, 21, or 25 contiguous nucleobases, wherein the targeting region is at least 90% complementary, at least 95% complementary, or is 100% complementary to an equal-length portion of an APP RNA having the nucleobase sequence of any of SEQ ID NOs: 1-7.

35.-42. (canceled)

43. The oligomeric compound of claim 5, wherein the oligomeric compound is a single-stranded oligomeric compound.

44.-52. (canceled)

53. An oligomeric duplex, comprising a first oligomeric compound comprising an antisense RNAi oligonucleotide of claim 34 and a second oligomeric compound comprising a sense RNAi oligonucleotide consisting of 17 to 30 linked nucleosides, wherein the nucleobase sequence of the sense RNAi oligonucleotide comprises an antisense-hybridizing region comprising least 15 contiguous nucleobases wherein the antisense-hybridizing region is at least 90% complementary to an equal length portion of the antisense RNAi oligonucleotide.

54. (canceled)

55. The oligomeric duplex of claim 53, wherein the sense RNAi oligonucleotide consists of 21 or 23 linked nucleosides.

56. The oligomeric duplex of claim 53, wherein 1-4 of the 3′-most nucleosides of the antisense RNAi oligonucleotide or the sense RNAi oligonucleotide are overhanging nucleosides.

57. The oligomeric duplex of claim 53, wherein 1-4 of the 5′-most nucleosides of the antisense RNAi oligonucleotide or the sense RNAi oligonucleotide are overhanging nucleosides.

58. The oligomeric duplex of claim 53, wherein the duplex is blunt ended at the 3′-end or at the 5′-end of the antisense RNAi oligonucleotide.

59.-66. (canceled)

67. The oligomeric duplex of claim 53, consisting of the antisense RNAi oligonucleotide and the sense RNAi oligonucleotide.

68.-76. (canceled)

77. A pharmaceutical composition comprising an oligomeric compound of claim 5 and a pharmaceutically acceptable carrier or diluent.

78. The pharmaceutical composition of claim 77, wherein the pharmaceutically acceptable diluent is artificial cerebral spinal fluid, sterile saline, or PBS.

79. (canceled)

80. (canceled)

81. A method of treating a disease associated with APP comprising administering to an individual having or at risk for developing a disease associated with APP a therapeutically effective amount of a pharmaceutical composition according to claim 77; and thereby treating the disease associated with APP.

82. The method of claim 81, wherein the disease associated with APP is Alzheimer's Disease, Alzheimer's Disease in a Down Syndrome patient, or Cerebral Amyloid Angiopathy.

83. The method of claim 81, wherein at least one symptom or hallmark of the disease associated with APP is ameliorated, wherein the symptom or hallmark comprises cognitive impairment, behavioral and psychological symptoms, gait disturbances seizures, progressive dementia, and/or abnormal amyloid deposits.

84. (canceled)
